(19) United States
(12) Patent Application Publication
(10) Pub. No.: US 2025/0357007 A1
(43) Pub. Date: Nov. 20, 2025

(54) SYSTEMS, APPARATUS, METHODS AND COMPUTER-ACCESSIBLE MEDIUM FOR PROVIDING HEALTH SYSTEM SCALE LANGUAGE MODELS WHICH CAN INCLUDE CLINICAL PREDICTION ENGINES

(71) Applicant: NEW YORK UNIVERSITY, New York, NY (US)

(72) Inventor: ERIC KARL OERMANN, Chapel Hill, NC (US)

(21) Appl. No.: 19/292,081

(22) Filed: Aug. 6, 2025

Related U.S. Application Data
(63) Continuation of application No. PCT/US2024/014563, filed on Feb. 6, 2024.
(60) Provisional application No. 63/443,584, filed on Feb. 6, 2023.

(51) Int. Cl. G16H 50/20; G16H 10/60

(52) U.S. Cl. CPC G16H 50/20; G16H 10/60

(57) ABSTRACT

Exemplary systems, methods, and computer-accessible medium are provided that that can implement and/or utilize clinical predictive models, which can assist physicians and administrators make decisions by forecasting clinical and operational events. Thus, the exemplary systems, methods, and computer-accessible medium are provided that convert clinical notes to training data using at least one natural language processing procedure, train a machine learning model using the training data finetune the trained machine learning model based on selected parameters, receive patient data, and generate at least one medical prediction on the received patient data with the trained finetuned machine learning model. Additional exemplary systems, methods, and computer-accessible medium are provided that can generate a table language by implementing an artificial intelligence model configured to generate code to create a structured database procedure. Further exemplary systems, methods, and computer-accessible medium are provided that can train an electronic health records (EHR) artificial intelligence model on a training data set that comprises a plurality of EHR records utilizing an under-sampling technique.

### CROSS REFERENCE TO RELATED APPLICATION(S)

This application relates to and claims the benefit of priority from U.S. Provisional Patent Application No. 63/443,584, filed on Feb. 6, 2023, the entire disclosure of which is incorporated herein by reference.

### FIELD OF THE DISCLOSURE

The present disclosure relates generally to a language model based systems and methods for processing medical records, and more specifically, to exemplary systems, methods and computer-accessible medium which can utilize, facilitate and/or provide exemplary language models that can integrate in real-time with clinical workflows centered around writing notes and placing electronic orders.

### BACKGROUND INFORMATION

Physicians make difficult decisions every day requiring the integration of a tremendous amount of information. One example is deciding when to discharge patients home from the hospital: a premature discharge could expose patients to excessive risk, and an inappropriate delay could limit the availability of hospital beds and potentially expose patients to the risk of hospital acquired conditions. The information for making these medical decisions is scattered in various records, e.g., the medical history, laboratory, and imaging reports. In performing their work, however, this information is ultimately integrated into the notes written by physicians to document and summarize patient care.

Clinical predictive models are frequently derived from rules that have existed for decades (see, e.g., Refs. [1-4]) as well as from machine learning methods (see, e.g., Refs. [5-7]), with most relying on structured inputs culled from the electronic health record or direct clinician inputs. This reliance on structured inputs introduces complexity in data processing, model development and deployment, which in part led to the overwhelming majority of medical predictive algorithms being trained, tested, and published, yet never deployed to assess their impact on real world clinical care. This can be referred to as the “last mile problem” (see, e.g., Refs. [8-10]).

One of the recent developments in modern artificial intelligence (AI) research is large language models (LLMs). These massive neural networks (millions or even billions of parameters) have been shown to obtain impactful results on a wide range of problems that rely upon the reading and interpretation of human language. Several types of LLMs have been developed over the past few years, broadly ranging from encoder models (such as BERT, i.e., see, e.g., Ref. [11]), and decoder models (such as GPT3, i.e., see, e.g., Ref. [12]). LLMs can be used to potentially solve this “last mile problem” in medical predictive analytics by simply reading the notes written by physicians, thereby immediately accessing a comprehensive description of patient's medical state to provide decision support at the point of care across a wide range of clinical and operational tasks. Nonetheless, the conventional use of the LLMs has not provided any such solutions.

Thus, it may be beneficial to provide an exemplary magnetic resonance system which can overcome at least some of the deficiencies described herein above.

### SUMMARY OF EXEMPLARY EMBODIMENTS

To solve the above-described problem and other related problems, exemplary systems, apparatus, method and computer-accessible medium according to the exemplary embodiment of the present disclosure can be provided (e.g., which can be labelled herein as “NYUTron” but not limited thereto), which can be include exemplary language-model based systems, apparatus, methods and computer-accessible medium that can integrate in real-time with clinical workflows centered around writing notes and placing electronic orders. Exemplary systems, apparatus, methods and computer-accessible medium accordingly to exemplary embodiments of the present disclosure can rely on and/or utilize the fact that all clinically useful data and medical professionals' decision-making process can be found as structured or unstructured text in electronic health records (e.g., notes, labs, reports on studies).

Exemplary systems, apparatus, methods and computer-accessible medium accordingly to exemplary embodiments of the present disclosure can utilize advances in natural language processing that provide that sufficiently-scaled self-supervised LLMs can outperform strongly supervised approaches on non-medical predictive tasks (see, e.g., Refs. [11-13]). For example, NYUTron can be assessed on a battery of five clinical and operational tasks and provide a detailed analysis of 30-day readmission task to look at questions of data efficiency, generalizability, deployability and potential clinical impacts. By reviewing medical predictive analytics (see Sect. 3.1 herein) as a natural language processing problem, exemplary systems, apparatus, methods and computer-accessible medium accordingly to exemplary embodiments of the present disclosure can facilitate the utilization of LLMs as universal prediction engines for a wide range of medical predictive tasks.

The following is intended to be a brief summary of the exemplary embodiments of the present disclosure, and is not intended to limit the scope of the exemplary embodiments.

In some exemplary embodiments of the present disclosure, exemplary systems, apparatus, methods, and computer accessible medium can be provided which can generate at least one medical prediction by converting clinical notes to training data using a natural language processing procedure, training a machine learning model using the training data, finetuning the machine learning model based on selected parameters, receiving patient data, and generating the at least one medical prediction on the received patient data with the trained and finetuned machine learning model.

Further, in some exemplary embodiments of the present disclosure, exemplary systems, apparatus, methods, and computer accessible medium, the clinical notes may include structured data and unstructured data. In addition, it is possible to integrate the machine learning model in real-time with clinical workflows, and may train the machine learning model using non-clinical data. According to various exemplary embodiments of the present disclosure, the medical prediction can include information associated with a readmission to a hospital, the clinical notes may include discharge notes, and/or, the finetuning may include replacing the trained machine learning model with a randomly initialized linear classifier after a last hidden layer of a pretrained BERT, which is a machine learning framework for natural language processing (NLP).

In some exemplary embodiments of the present disclosure, exemplary systems, methods, and computer accessible medium can be provided which can generate a table language by implementing an AI model configured to generate code to create a structured database procedure.

Additionally, in some exemplary embodiments of the present disclosure, the code generated by the AI model to create the structured database procedure can convert unstructured text into a plurality of SQL tables, and the unstructured text can comprise electronic health records free text.

In some exemplary embodiments of the present disclosure, exemplary systems, apparatus, methods, and computer accessible medium can be provided which can train an electronic health records (EHR) artificial intelligence model on a training data set comprising a plurality of EHR records utilizing an under-sampling technique, where the under-sampling technique can be an iterative summation, a hierarchy, and/or a sparse-attention model.

For example, in the case of iterative summation, exemplary systems, methods, and computer accessible medium can select a fixed amount of data from a selected one of the plurality of EHR records, summarize information in the fixed amount of data, select a next fixed amount of data from the selected HER record, feed the summary and the next fixed amount of data back into the EHR artificial intelligence model, and create an updated summary based on the summary and next fixed amount of data.

with respect to a hierarchy, exemplary systems, apparatus, methods, and computer accessible medium may select first fixed amount of data from a selected one of the plurality of EHR records, convert the first fixed amount of data into a machine language, select a second fixed amount of data from the selected HER record, and convert the second fixed amount of data into a machine language that is added to the machine language for the first fixed amount of data.

For a sparse-attention model, exemplary systems, apparatus, methods, and computer accessible medium may select a word sampling rate for the plurality of EHR records, apply the word sampling rate to the plurality of EHR records, and train the EHR artificial intelligence model on the plurality of EHR records subject to the word sampling rate.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1(a)-1(d) are exemplary illustrations of exemplary configurations of the exemplary language-model based model for clinical prediction according to an exemplary embodiment of the present disclosure;

FIGS. 2(a)-2(c) are exemplary illustrations of an exemplary overall temporal-test performance across five exemplary tasks according to exemplary embodiments of the present disclosure;

FIGS. 3(a)-3(c) are exemplary illustrations of exemplary results of an exemplary retrospective study of an exemplary readmission prediction according to exemplary embodiments of the present disclosure;

FIGS. 4(a) and 4(b) are exemplary illustrations of the exemplary prospective study of the exemplary predictive performances according to exemplary embodiments of the present disclosure;

FIG. 5 is a flow diagram of an exemplary method providing an exemplary decision tree which has levels for predicting a readmission according to exemplary embodiments of the present disclosure;

FIGS. 6(a) and 6(b) are exemplary illustrations of examples and a visualization of an exemplary dataset according to an exemplary embodiment of the present disclosure;

FIGS. 7(a) and 7(b) are exemplary illustrations of an exemplary readmission wordcloud and the impact of note length on readmission prediction according to exemplary embodiments of the present disclosure;

FIGS. 8(a) and 8(b) are exemplary graphs illustrating detailed statistics of the comparison between language models and lace+xgb according to exemplary embodiments of the present disclosure;

FIGS. 9(a) and 9(b) are exemplary graphs illustrating exemplary difference between random test and temporal test according to exemplary embodiments of the present disclosure;

FIGS. 10(a) and 10(b) are exemplary graphs illustrating of exemplary benchmarking the exemplary model against a traditional NLP model and other language models on a different clinical prediction task (clinical concept extraction) according to exemplary embodiments of the present disclosure;

FIGS. 11(a) and 11(b) are exemplary graphs showing exemplary model's calibration curve for a temporal test and a prospective deployment according to exemplary embodiments of the present disclosure;

FIGS. 12(a) and 12(b) are exemplary graphs/charts providing an exemplary bias analysis stratifying the exemplary model's performance by clinical departments and months according to exemplary embodiments of the present disclosure;

FIGS. 13(a) and 13(b) are exemplary graphs/charts providing the exemplary bias analysis stratifying the exemplary model's performance by age groups and major racial groups according to exemplary embodiments of the present disclosure;

FIG. 14 is an exemplary graph showing a comparison of the exemplary model's and BioClinicalBERT's performance on MIMIC-III Readmission according to an exemplary embodiment of the present disclosure;

FIG. 15 is a set of exemplary scatterplots for F1, precision, and recall for of ClinicalBERT's performance changes (Y axis) on labels of procedure ICD-9, when DRG is added as the auxiliary task, versus the balances (X axis) of the labels, and versus the correlations (sizes and colors of the units) between each label with the whole auxiliary DRG task according to an exemplary embodiment of the present disclosure;

FIG. 16 is an exemplary bar chart illustrating the distribution of lengths of tokenized discharge summaries in a MIMIC-III dataset according to an exemplary embodiment of the present disclosure;

FIG. 17 is an exemplary graph illustrating the distribution of diagnosis ICD-9 according to an exemplary embodiment of the present disclosure;

FIG. 18 is an exemplary graph illustrating the distribution of procedure ICD-9 according to an exemplary embodiment of the present disclosure;

FIG. 19 is an exemplary graph illustrating the performance of ClinicalBERT on different text sections and different types of notes according to an exemplary embodiment of the present disclosure;

FIG. 20 is an exemplary bar chart illustrating the performance of ClinicalLongformer on different text sections and different types of notes according to an exemplary embodiment of the present disclosure;

FIG. 21 is an exemplary bar chart illustrating the performance of ClinicalBERT and ClinicalLongformer on clinical note combinations according to an exemplary embodiment of the present disclosure; and

FIG. 22 shows a block diagram of an exemplary embodiment of a system according to the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The following description of exemplary embodiments provides non-limiting representative examples referencing numerals to particularly describe features and teachings of different exemplary aspects and exemplary embodiments of the present disclosure. The exemplary embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the exemplary embodiments of the present disclosure to such an extent that other implementations, not specifically covered but within the knowledge of a person of skill in the art having read the description of embodiments, would be understood to be consistent with an application of the exemplary embodiments of the present disclosure.

### 1.1 Exemplary Language-Model Based Approach to Clinical Prediction

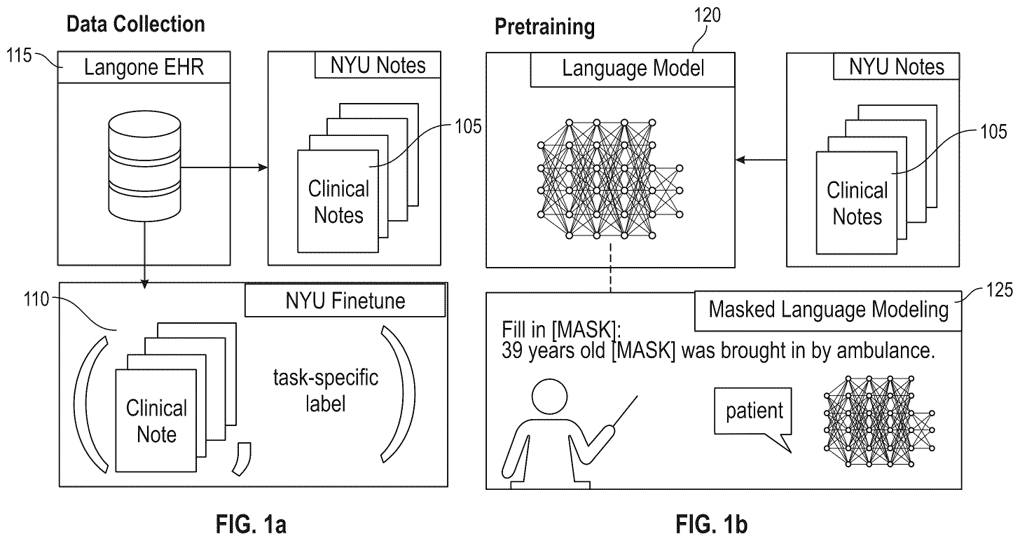

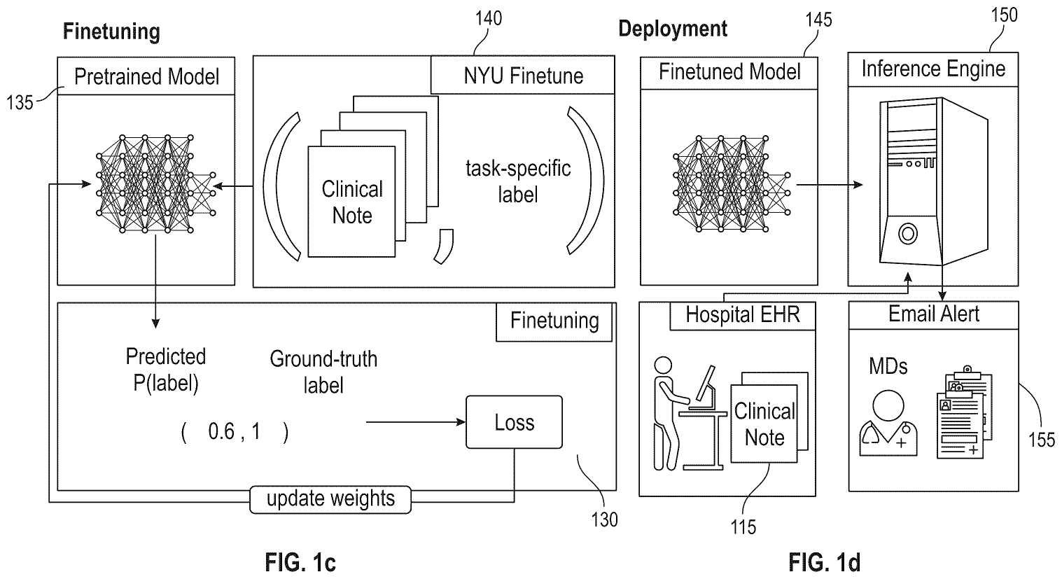

Exemplary systems, apparatus, methods and computer-accessible medium accordingly to an exemplary embodiment of the present disclosure can be or include a language-model based approach or model which can have certain exemplary steps, e.g., data collection, pretraining, finetuning, and deployment. FIGS. 1(a)-1(d) provides illustrations of an overview of the exemplary language-model based approach for clinical prediction according to an exemplary embodiment of the present disclosure.

For example, in the first step shown in FIG. 1(a), exemplary systems, methods, apparatus and computer-accessible medium accordingly to an exemplary embodiment of the present disclosure collected a vast set of unlabeled clinical notes and five task-specific labelled set of clinical notes from the NYU Langone EHR. Unlike prior situations, exemplary datasets come from the entire hospital system with a diverse patient population from different clinical departments. The exemplary large unlabeled dataset, “NYU Notes”, comprises about 7.25 million clinical notes (e.g., radiographic reads, history and physicals) from 336,000 patients across four hospitals, resulting in a 4.1 billion word corpus curated from January 2011 to May 2020. Each one of the exemplary labelled finetuning sets contains 1-10 years of inpatient clinical notes (55,791-413,845 patients, 51-87 million words) with task-specific labels (2-4 classes). See Table 7 for exemplary dataset statistics.

In the second step and the third step shown in FIGS. 1(b) and 1(c), respectively, the exemplary LLM was pretrained and fine-tuned for each downstream task using a bidirectional encoder model known as BERT (Bidirectional Encoder Representation with Transformer) and a masked language modeling (MLM) objective on the NYU Notes dataset (see, e.g., Ref. [11]) until the validation loss plateaued. The exemplary MLM objective randomly masks out words or subwords in clinical notes and trains the language model to fill in the masked word correctly. Next, using the finetuning dataset, the exemplary pretrained model was finetuned (herein termed “NYUTron”) to predict the task label using the relations learned in pretraining with clinical notes.

In the fourth step shown in FIG. 1(d), the exemplary model was deployed to a high-performance inference engine, NYUTriton, that interfaces with the NYU Langone EHR. The deployment facilitates real-time LLM guided inference at the point of care. In a single-armed, non-interventional, prospective trial, NYUTron's performance was validated on 30-day readmission prediction in a real-world environment and assessed its potential clinical impacts.

### 1.2 Exemplary Overall Performance on Five Exemplary Tasks

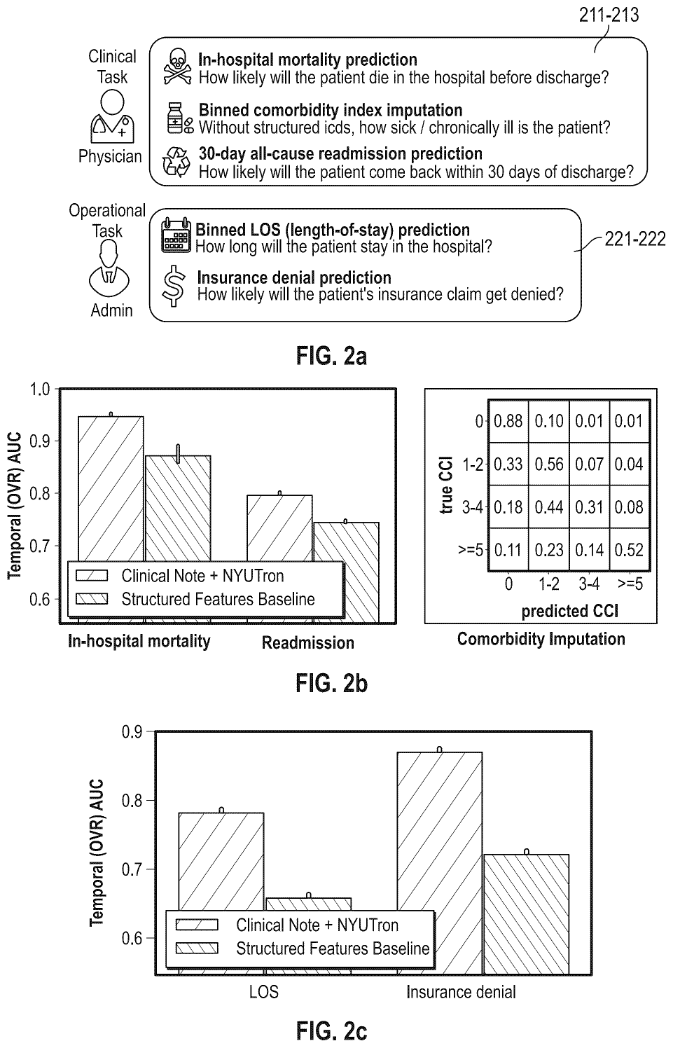

To assess the breadth of NYUTron's applicability, NYUTron's performance was evaluated on five tasks, retrospectively (with detailed descriptions of exemplary datasets provided in section 2.1.2). The full dataset was trained and evaluated with two test sets: (1) a random test set (e.g., clinical notes sampled from the same time as the train data) and (2) a temporal test set (e.g., clinical notes sampled from the future of train data). The temporal test set resembles the deployment scenario more, where the inference data comes from the future of the training data. FIGS. 2(a)-2(c) provide illustrations of an exemplary overall temporal-test performance across five tasks according to exemplary embodiments of the present disclosure.

The exemplary battery of tasks can include, e.g., three tasks (211)-(213) and two operational tasks (221)-(222), as shown in FIG. 2(a). NYUTron is compared against structured baselines, which forward structured features used by traditional clinical predictive models into an extreme gradient boosted tree model (see, e.g., Ref. [14]). Additional details are provided herein in section 2.6.

The exemplary NYUTron can extend to multiple clinical and operational tasks. FIGS. 2(b) and 2(c) show that on the prediction tasks (in-hospital mortality, readmission, LOS, insurance denial), NYUTron can have an AUC of 78.7%-94.9%, with an improvement of 5.36%-14.7% AUC from traditional clinical predictive models. On the comorbidity imputation task, the exemplary NYUTron can have a median AUC of 89.4%±0.275%. the present disclosure first present our results across four of the tasks, and conclude with focused look at readmission prediction that addresses questions of data efficiency, model generalizability, and deployment in a real world environment.

The exemplary NYUTron can predict risk of in-hospital mortality on admission and imputing comorbidity index. The task of in-hospital mortality prediction is to estimate (at admission) the likelihood of a patient's death during the present inpatient encounter. FIG. 2(b) shows that for in-hospital mortality prediction, NYUTron has a median AUC of 94.9%±0.168% with a 7.43% improvement from its structured baseline based on SAPS2 (see, e.g., Ref. [15]) and APACHE2 (see, e.g., Ref. [16]) features such as age and mean heart rate, asl also discussed herein. The task of comorbidity index imputation is to predict (at admission) the likely Charlson Comorbidity Index (CCI) (see, e.g., Ref. [17]) with no available structured features for chronic diseases. The exemplary embodiments framed this as a data imputation problem, as 22% of the dataset lacked CCI scores and this was known area for documentation improvement; see supplementary 3.10 for more context). Systems, methods, apparatus and computer-accessible medium according to exemplary embodiments of the present disclosure discretized the index into 4 bins according to the original paper's grade of severity (none: 0, mild: 1-2, moderate: 3-4, severe: ≥5). FIG. 2(b) shows that, e.g., on comorbidity imputation, NYUTron has a median AUC of 89.4%±0.275% and a 88% precision of identifying patients whose CCI is 0.

The exemplary NYUTron can be used for operational endpoints and predict in-patient length of stay and insurance claims denial on admission. The task of length-of-stay prediction is to predict (at admission) the likely range of days a patient will stay in the hospital. Exemplary embodiments discretized the length of stay into 4 bins (0-25% quantile, 25-50% quantile, 50%-75% quantile, 75%+). FIG. 2(c) shows exemplary illustrations which provide for length-of-stay prediction, and NYUTron has an median one-versus-rest AUC of 78.7%±0.179% with 12.3% improvement from the structured baseline, which uses an available subset of “Lisbon Portugal” features as in [18]. The task of insurance claim denial is to predict (at admission) whether the insurance claims submitted for this encounter will be accepted or initially denied. FIG. 2(c) shows that for insurance denial prediction, NYUTron has an median AUC of 87.2%±0.246% with 14.7% improvement from the structured baseline, which uses an available subset of “claim form” features in [19] such as age and insurance brand. Exemplary NYUTron can also predict different types of denials from both admission notes and discharge notes with similar performance, as further discussed herein in section 3.2.

### 1.3 Exemplary Detailed Analysis on Readmission Prediction

To further understand NYUTron's performance, systems, methods, apparatus and computer-accessible medium according to exemplary embodiments of the present disclosure performed a detailed analysis of 30-day all-cause readmission prediction. The exemplary task of readmission prediction is to predict (at discharge) the likelihood of the patient coming back to the hospital within 30 days, and is a well-studied problem in the medical informatics literature. Addition details regarding the readmission task are discussed herein in section 3.3.

FIG. 2(b) shows that for 30-day all-cause readmission prediction, the exemplary NYUTron has a median AUC of 79.87%±0.168% with a 5.36% improvement from its structured baseline, which uses LACE (see, e.g., Ref. [20]) features such as length-of-stay and acuity of admission. Systems, methods, apparatus and computer-accessible medium according to exemplary embodiments of the present disclosure added, e.g., 5 evaluations in both retrospective and prospective settings: (1) a human comparison with 6 attending physicians on predicting 20 patient cases sampled from the random split, (2) a study on NYUTron's scaling properties with respect to data by comparing NYUTron and other models using different number of finetune data, (3) an assessment of NYUTron's cross-site generalizability using pretraining, finetuning and test data from different locations, (4) a prospective, single-arm, non-interventional study to evaluate NYUTron's deployability, and (5) a physician panel's qualitative evaluation of NYUTron's prospective performance to assess clinical impacts.

### 1.3.1 Exemplary Retrospective Study of Readmission Prediction

On small samples, exemplary NYUTron can be competitive with a small group of physicians at predicting 30-day readmissions. Exemplary embodiments tested a group of 6 physicians at different levels of seniority against an exemplary NYUTron in a head to head comparison to establish a baseline difficulty for predicting 30-day all cause readmission at time of discharge (See method 2.8.2 for details).

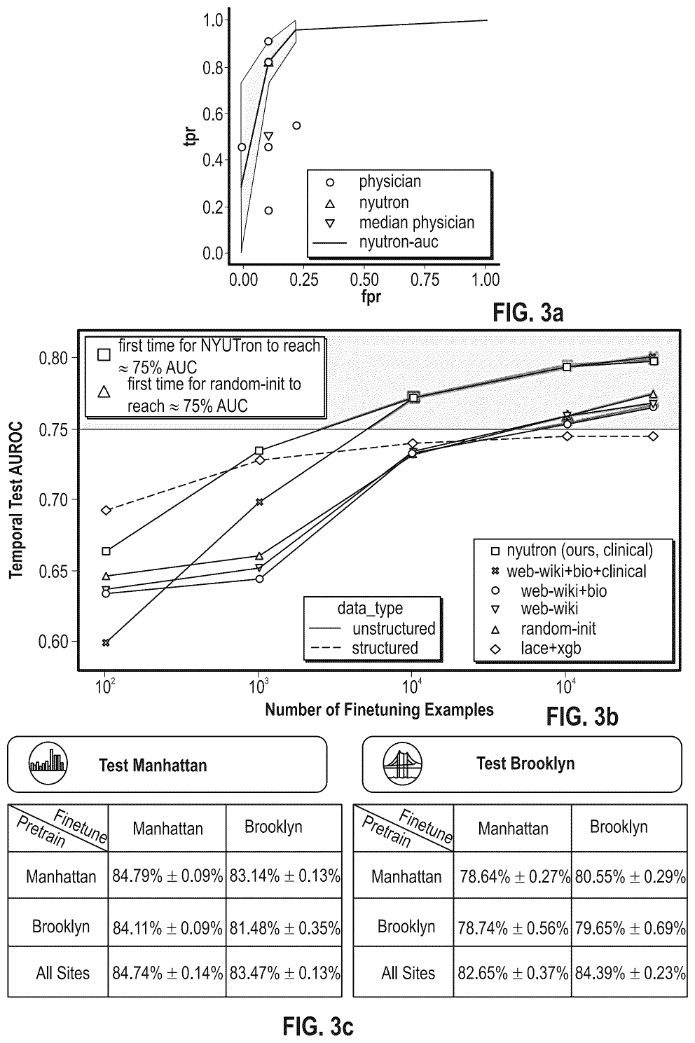

FIGS. 3(a)-3(c) provide exemplary illustrations of an exemplary retrospective study of exemplary NYUTron's readmission prediction according to exemplary embodiments of the present disclosure.

For example, discharge summaries (N=20, 11 positive cases and 9 negative cases) were sampled from the random split and uploaded to an online evaluation platform. Median physician performance was worse than NYUTron (FIG. 3(a)). The median physician and NYUTron have a FPR of 11.11%, while the median physician has a TPR of 50% compared to NYUTron's TPR of 81.82%. Physicians have a median F1-score of 62.8% and a substantial variance of 22.2% compared to NYUTron's F1-score of 77.8%.

For 20 cases sampled from the random split, NYUTron's true positive rate (TPR) and false positive rate (FPR) were compared with 6 physicians. NYUTron (orange upper triangle) has a higher TPR and the same FPR compared to the median physician performance (green circle).

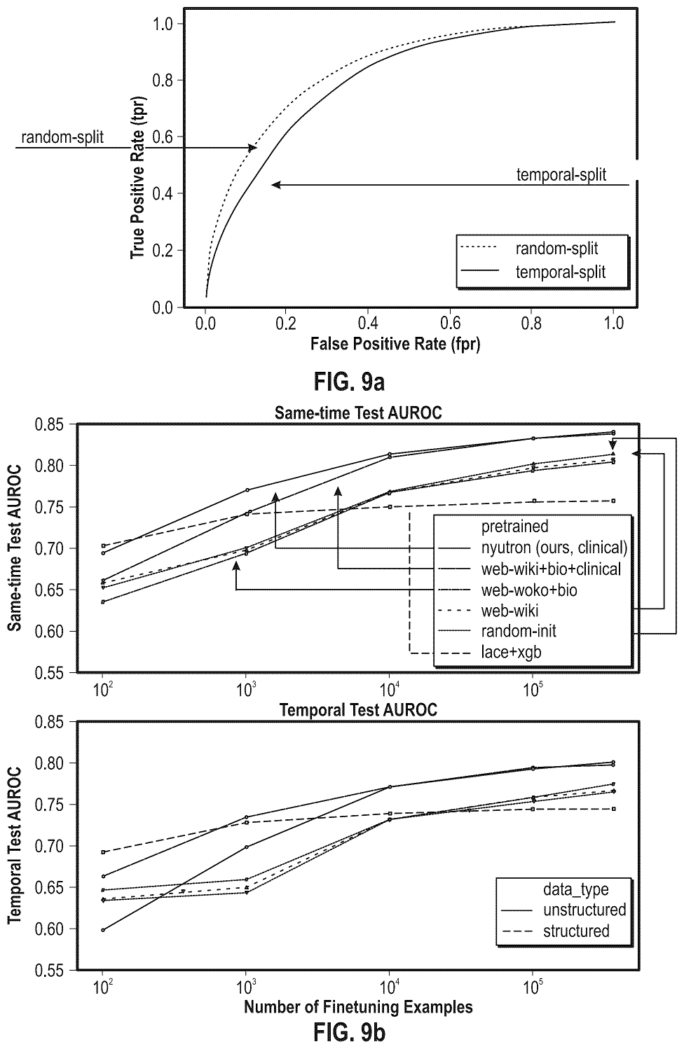

The random split does not resemble the deployment scenario, where the test data comes from the future of the training data. Exemplary embodiments therefore created a temporal split to simulate deployment, and observed a meaningful difference of test statistics against the random split (random test AUC is 84.13%, whereas temporal test AUC is 80.2%) confirming the importance of this second testing phase. See Extended Data FIG. 9 for more details.

FIGS. 9(a) and 9(b) shows graphs illustrating the exemplary difference(s) between random test and temporal test according to exemplary embodiments. In particular, FIG. 9(a) illustrates a graph of an AUC curve for the random test which shows better performance than temporal test. The random-test AUC is 84.13%, compared to the temporal-test AUC of 80.2%. The difference highlights the importance of creating a test set to reflect the problem setup. In the case of readmission prediction, the deployment set always come from the future of the training set. Thus, it is possible to use the temporal test AUC for model selection.

FIG. 9(b) illustrates a graph of a comparison of random-test AUC and temporal-test AUC as the number of training examples increases. This graph of FIG. 9(b) shows that temporal-testing is important to estimate deployment performance, and also that sampling a temporally split out dataset seems “harder” than a randomly sampled test dataset because all tested LLMs and lace+xgb perform worse on the temporal test (e.g., notes from the future) than the random test (e.g., notes from the same time as the training data). The lines on the left (e.g., random test AUCs) are generally higher than the colored lined on the right (e.g., temporal test AUCs). It is possible to conclude that this is an important distinction that temporally sampled held-out test sets give a more realistic estimate of model performance. Interestingly, the language models appear to be more sensitive to this phenomenon than the lace+xgb model.

The exemplary NYUTron can be competitive with and an improvement of traditional models and other LLMs. The effectiveness of NYUTron was evaluated by comparing its test performance on the temporal split against a traditional model and four different types of LLMs as also discussed in sections 2.6 and 2.8.3 herein. NYUTron has the highest AUC when finetuned with the full dataset (see FIG. 3(b)) with a median AUC of 79.87%±0.17%, which is similar to clinical+web-wiki+bio's AUC of 80.14%±0.26%. Compared to LLMs pretrained with nonclinical texts (e.g., web-wiki+bio and web-wiki), NYUTron's median AUC is 2.37% to 3.23% higher. Compared to the traditional model that uses structured features (e.g., lace+xgb), NYUTron has a 5.36% higher AUC. Compared to the model that uses traditional NLP embedding (e.g., tf-idf+xgb), NYUTron has a 12.8% higher median AUC (See Extended Data 10a for more details).

For example, a comparison of temporal test AUCs of different pre-trained LLMs with an increasing amount of finetuning examples is illustrated in a graph of FIG. 3(b). For the sake of simplicity, the variances is omitted and only the median performance of 5 trials is plotted. The exemplary comparison of median performances with 100 and 1000 examples is less significant because AUCs with sparse finetuning examples have high variances (at 100 examples, 4.26% to 9.56% variance is shown/provided; at 1000 examples, 0.44% to 9.46% variance is shown and/or provided. Variances of AUCs decrease with more finetuning examples).

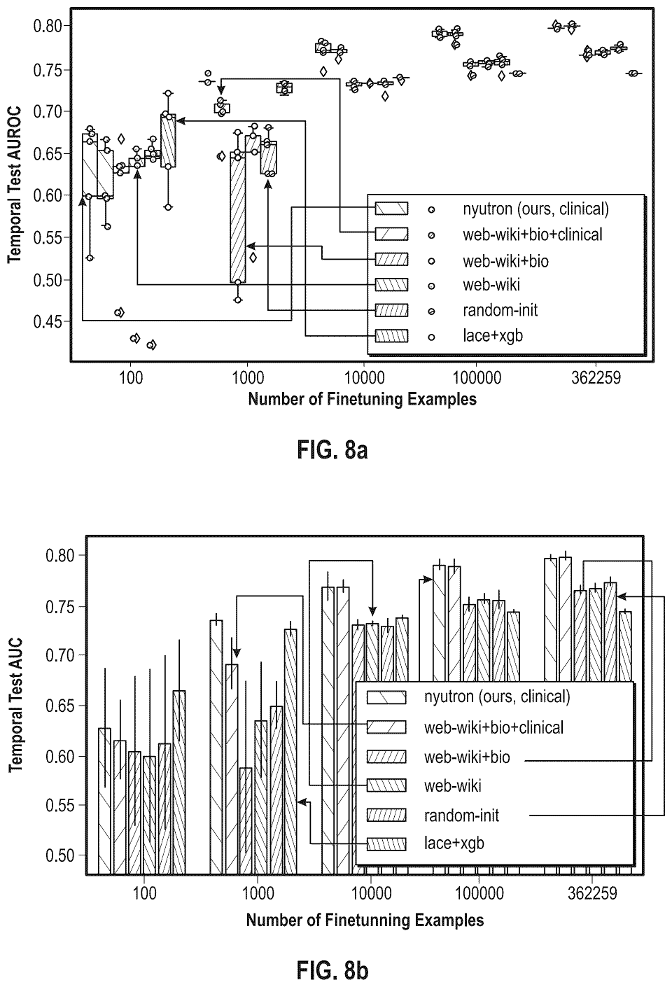

Further, FIGS. 8(a) and 8(b) illustrate graphs illustrating exemplary detailed statistics of the comparison between language models and lace+xgb according to exemplary embodiments. FIG. 8(a) shows an exemplary barplot that shows the mean and standard deviation. The height of the bar indicates the mean across 5 experiments and the length of the black vertical line indicates the standard deviation. FIG. 8(b) shows an exemplary boxplot with individual data points. For each model, 5 experiments were run using random seeds 0, 13, 24, 36, 42. The center line of the box plot indicates the median. The upper line of the box indicates first quantile. The lower line of the plot indicates the last quantile. The whisker extends to 1.5 times the interquartile length and the diamonds indicate outliers.

A LLM trained on unstructured clinical notes better scales with data compared to traditional structured models. Compared to lace+xgb, NYUTron benefits from an increasing amount of labelled examples and achieves a better AUC when finetuned with the full dataset. FIG. 3(b) shows that lace+xgb (dashed yellow line) and NYUTron (solid green line) have similar AUCs at 100 and 1000 examples. However, NYUTron's AUC consistently improves with more examples while lace+xgb's AUC starts to plateau (From 100 to 1000 examples, NYUTron's AUC increases 7.27% while lace+xgb increases 3.98%; From 10,000 to 392,336 examples, NYUTron's AUC increases 2.15% while lace+xgb's AUC increases 0.63%). With the full finetuning dataset, NYUTron has a 7.04% higher AUC than lace+xgb.

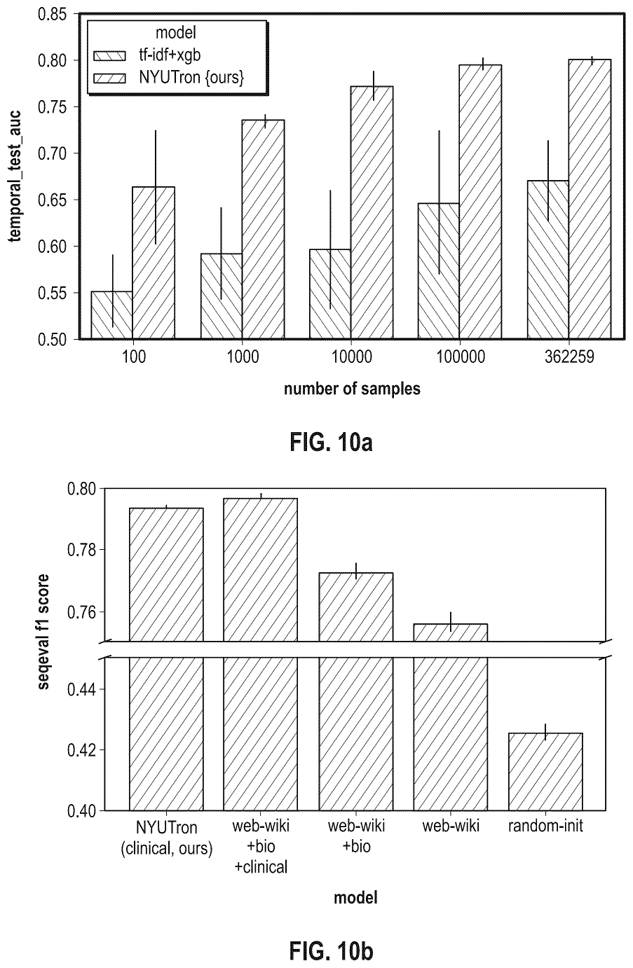

FIGS. 10(a) and 10(b) illustrate exemplary graphs providing an exemplary benchmarking NYUTron against a traditional NLP model and other language models on a different clinical prediction task (e.g., clinical concept extraction) according to exemplary embodiments. Similar trend as readmission prediction are observed: In general, FIG. 10(a) shows that NYUTron has better performance than tf-idf under different data availability settings, and FIG. 10(b) shows that clinically pretrained language models have better performance than non-clinically pretrained language model. This corroborates the findings that health-system scale language models are general purpose clinical pre-diction engines and that a domain match between pretraining and finetuning corpus contributes to task performance.

In particular, the graph of FIG. 10(a) shows an exemplary comparison of temporal test AUCs between NYUTron and a traditional NLP model (tf-idf+xgb). NYUTron has a higher median AUC than tf-idf+xgb for all tested number of finetuning examples. The black vertical line indicates standard deviation over 5 trials of different random seeds (0, 13, 24, 36, 42). The graph of FIG. 10(b) shows an exemplary comparison of LLMs' finetuning performances on the NER task. On the i2b2-2012 clinical concept extraction task, the LLMs that are pretrained with clinical corpora (NYUTron, web-wiki+bio+clinical) have a higher average f1 score than LLMs that are not pretrained with clinical corpora (web-wiki+bio, web-wiki, random-init). For example, NYUTron and web-wiki+bio+clinical perform better than the randomly initialized model (36.64% higher median seqeval f1 score) and non-clinically pretrained models (2.01%-3.48% higher median seqeval f1 score). For example, the height of each bar is the average f1 score and the length of each black vertical line indicates the standard deviation of the f1 scores.

Pretraining on a large amount of unlabeled clinical notes con-tributes to performance. Compared to the randomly initialized LLM, NYUTron learns to generalize better from fewer examples. Turning back to FIG. 3(b), this figure shows that while NYUTron needs 10,000 examples to achieve around 75% AUC, random-init needs 100,000 examples. It was also observed that a similar trend in another clinical prediction task, Extended Data, FIG. 10(b) shows that NYUTron per-forms better than the randomly initialized model (e.g. 36.83% higher F1 score) and the non-clinically pretrained models (2.06% to 3.73% higher F1 score) on the clinical named entity recognition (NER) task from the 2012 i2b2 challenge.

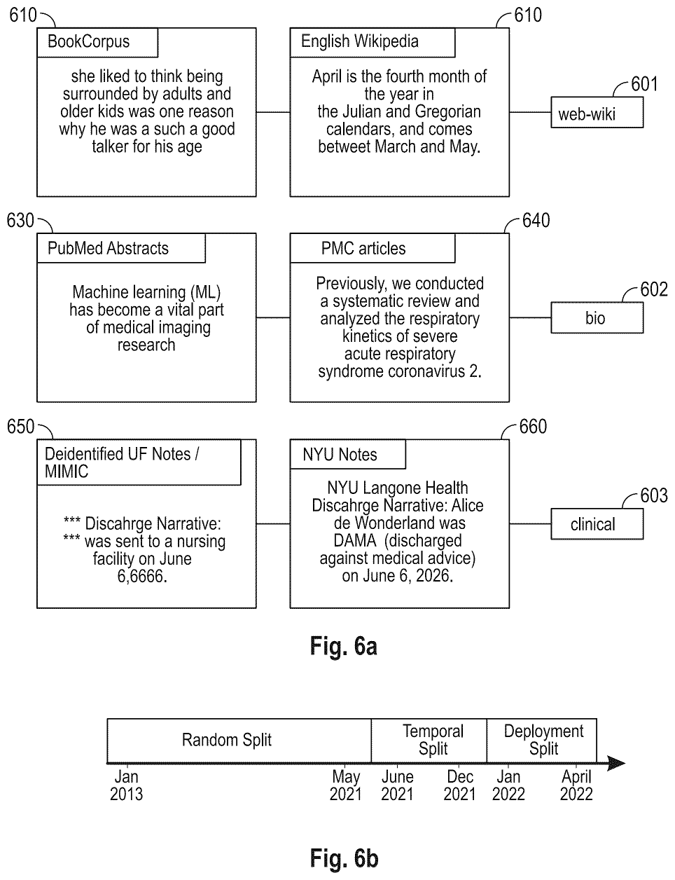

It can be beneficial to match the domain of the pretraining corpus and the domain of the finetuning corpus. Indeed, the illustration of FIG. 3(b) provides certain exemplary evidence: LLMs pretrained on nonclinical texts (web-wiki and web-wiki+bio) have similar performances as random-init. A separate LLM, web-wiki+bio+clinical, has a similar performance as NYUTron. Third, Compared to LLMs pre-trained on nonclinical texts (web-wiki, web-wiki+bio), clinically pretrained LLMs (NYUTron, web-wiki+bio+clinical) learn to generalize better from fewer examples. (See, e.g., Extended Data Table 6, and FIG. 6(a) for dataset statistics and examples of pretrain corpus).

For example, FIGS. 6(a) and 6(b) provided illustrations of examples and visualization of an exemplary dataset according to an exemplary embodiment. In particular, FIG. 6(a) shows examples of pretraining corpora, including three types of pretrain corpus: (601) web-wiki (online books from bookcorpus (see, e.g., Ref. [38]) and encyclopedia articles from English Wikipedia (see, e.g., Ref. [39])), (602) bio (abstracts of academic papers from Pubmed Abstracts (see, e.g., Ref. [40]) and full articles from Pubmed Central (see, e.g., Ref. [41])), and (603) clinical (NYU Notes, NYU Readmission from Langone EHR and clinical notes from University of Florida Health).

FIG. 6(b) shows an exemplary visualization of exemplary readmission data split timelines. This example visualizes the random split, temporal split, and deployment split on a timeline to indicate this decision for model evaluation. The random split starts from January 2013 and ends at May 2021 (inclusive), which is further split into a 80% train set, 10% validation set and a 10% test set. The temporal split (temporal test) starts from June 2021 and ends at December 2021, a time period from which no training samples were sampled from. The deployment data is necessarily sampled from the future as it is accrued prospectively as part of our single arm, non-interventional clinical trial.

Having a close domain match during pretraining is particularly beneficial in the low data setting during finetuning. Two language models were compared that were pretrained on clinical text from different hospital systems, NYUTron and web-wiki+bio+clinical. Turning to FIG. 3(b), this figure shows that at 1,000 examples, NYUTron (the in-domain model) has a higher AUC for NYU Readmission than web-wiki+bio+clinical (the out-of-domain model). Notably, NYUTron's advantage disappears as the number of finetuning examples increases, suggesting that sufficient in-domain finetuning can adapt models that were pretrained out-of-domain.

Clinical language models show generalizability to different sites through local finetuning. In order to investigate the robustness of NYUTron across clinical environments, two hospitals that are geographically separated within the NYU Langone Health System were chosen. For brevity, Tisch Hospital in Manhattan is referred to as “Manhattan”, NYU Langone Hospital—Brooklyn is referred to as “Brooklyn”, and all four hospitals within the NYU Langone Health System (Manhattan, Brooklyn, NYU Langone Orthopedic Hospital, NYU Langone Hospital—Long Island) are refereed to as “All Sites”. Three LLMs pretrained on different sites: the first one is pretrained in Manhattan, the second one is pretrained in Brooklyn, and the third one is pretrained in all sites. For each of the pretrained LLM, exemplary embodiments finetune it with a readmission dataset from either Manhattan or Brooklyn. Finally, the systems, methods, apparatus and computer-accessible medium according to the exemplary embodiments of the present disclosure ask the finetuned LLM to predict readmission based on discharge notes from either Manhattan or Brooklyn.

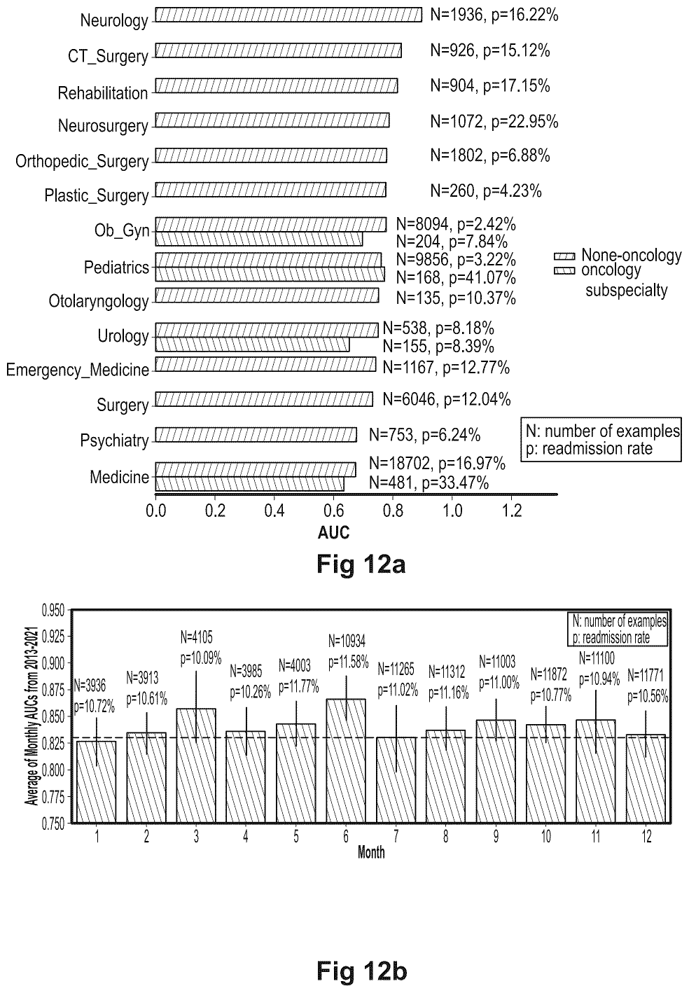

FIG. 3(c) shows an exemplary illustration that the LLM pretrained on all sites have the best performance on both the “Test Manhattan” and “Test Brooklyn”. For all of the pretrained LLMs, finetuning with the local dataset (“Finetune Manhattan/Brooklyn”) leads to a higher test AUC in the test site (“Test Manhattan/Brooklyn”) compared to finetuning at another site (“Finetune Brooklyn/Manhattan”). Therefore pretraining with data from all sites and local finetuning is the best way to optimize performance. Additional analysis was performed in the supplemental (3.6 discusses generalization to a different health system through finetuning and 3.7 compares the robustness of NYUTron and lace+xgb with respect to training sites). It was found that NYUTron is sensitive to notes from different clinical departments, patient demographics, and that its performance fluctuates over months (Extended Data FIG. 12a,13,12b). The causes of the discrepancies can be complex (as discussed in section 3.9 herein).

### 1.3.2 Exemplary Prospective Study of Readmission Prediction

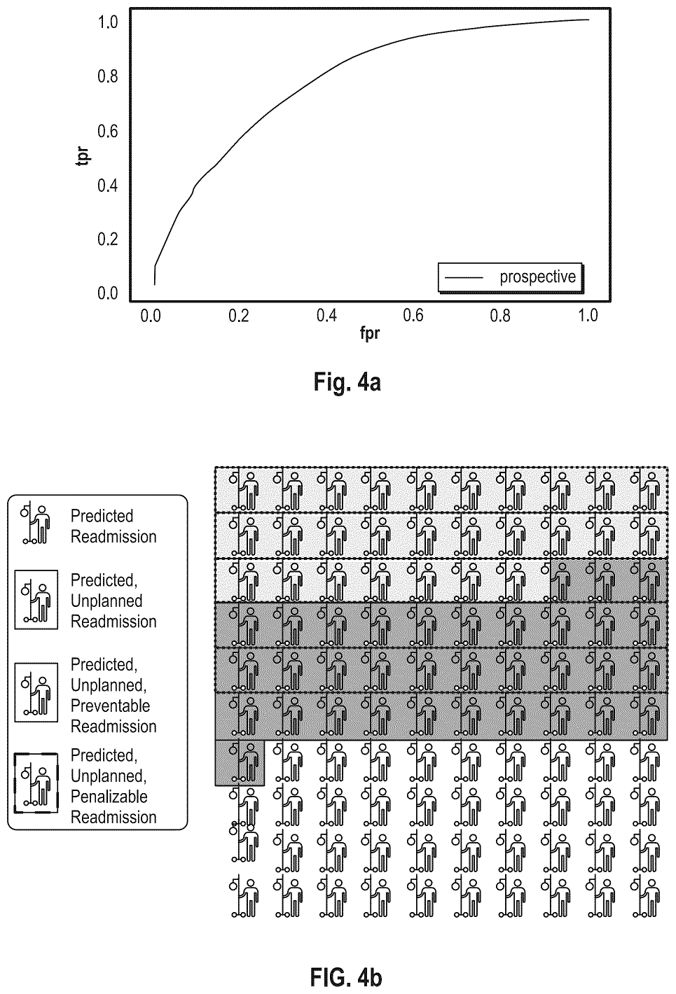

To assess NYUTron's performance outside the development environment, an exemplary model was selected based on the retrospective trial results and ran a prospective trial from January to April 2022. FIGS. 4(a) and 4(b) show exemplary illustrations of an exemplary prospective study of NYUTron's predictive performances according to exemplary embodiments of the present disclosure.

For example, exemplary NYUTron was deployed in an accelerated format and loaded it into an inference engine which interfaces with the her to read discharge notes as they are signed by the treating physicians. There were 29,286 discharged encounters, of which 3,271 patients (11.17%) came back within 30 days. NYUTron predicted 2,692 out of the 3,271 readmissions (82.30% recall) with 20.58% precision. FIG. 4(a) shows that NYUTron has an AUC of 78.70%.

To determine the potential clinical impact, a group of 6 physicians performed a qualitative evaluation of 100 randomly sampled readmitted cases that were captured by NYUTron upon the trial's conclusion. Small-sample physician review suggests that some true positive predictions by NYUTron are clinically significant, preventable readmissions. Overall, the readmitted patients who are predicted to be readmitted are 6.02 times more likely to die in-hospital and stay 2.93 days longer (p<10-4). For example, 6 physicians were asked to manually review 100 true positive cases to assess preventability and clinical impact. As shown in FIG. 4(b), about 61% of the predicted cases (blue box) are unplanned, whose mean predicted probabilities are lower than those of planned readmissions (e.g., 31.9%±31.1% vs. 82.1%±27.3%; p<10-4). Among the unplanned readmissions, 19.67% of patients experienced an adverse event or death on readmission, with 50% of those events considered preventable by the physician panel. From a financial standpoint, 81.9% of the unplanned readmissions were considered penalties by CMS guidelines (underlined icons). Among the penalizable cases, about 54% are considered preventable (red icons). Notably, three out of the 27 preventable readmissions had Clostridioides difficile enterocolitis, a contagious, healthcare-associated bacterial infection that causes one in 11 people over age 65 to die within one month (see, e.g., Ref. [21]). For more details on the physician review see method 2.8.6.

In FIGS. 4(a) and 4(b), the illustrations of a further exemplary embodiment of NYUTron's predictive performances are provided. In the illustration of FIG. 4(a), NYUTron has an AUC of 78.70% in a prospective, single-arm, non-interventional trial with a recall of 82.3% at a precision of 20.6%. In the illustration of FIG. 4(b), a panel of six physicians reviewed NYUTron's results for potential clinical impact. For every 100 readmitted patients who are successfully identified by NYUTron, 61% are unplanned readmissions, 50% would result in a fine under CMS guidelines, and 27% are preventable at time of discharge according to the consensus opinion of a multispecialty panel of physicians who reviewed cases from the prospective trial. See supplemental 3.3.3 for a discussion on the readmission label and 3.3.4 for the practical significance of the performance.

### 1.4 Exemplary Discussion

Exemplary systems, methods, apparatus and computer-accessible medium accordingly to various exemplary embodiments of the present disclosure can relate to developing, training, validating, and deploying NYUTron, an exemplary health-system, apparatus, method and computer accessible medium which can scale LLM designed and validated for clinical use. Exemplary NYUTron can perform on three clinical tasks (in-patient mortality prediction, comorbidity prediction, readmission prediction) and two operational tasks (insurance claims denial prediction, in-patient length of stay prediction). The systems, methods, apparatus and computer-accessible medium accordingly to an exemplary embodiment of the present disclosure performed a detailed analysis of readmission prediction due to its clinical and operational significance, and its well documented history in the medical informatics literature. Exemplary embodiments can offer flexibility in using an encoder architecture (BERT), which only relies on unstructured text inputs to generate a single prediction, as being a virtue of the exemplary embodiments.

An ethical consideration in deployment can be that physicians may over-rely on NYUTron's predictions due to its seamless integration with existing medical work flows thereby leading to undesirable medical outcomes. Further research can optimize human-AI interactions to prevent over-dependence on clinical language models as well as developing standardized assessments for sources of bias or other unexpected failure points. The systems, methods, apparatus and computer-accessible medium accordingly to an exemplary embodiment of the present disclosure can measure the alignment of language model's sensitivity patterns with physicians through token-level perturbations of the clinical notes (see, e.g., Ref. [22]).

Large, generative LLMs also present a unique opportunity for integration into medical workflows, however they are highly dependent on user inputs and prompting, and not suitable for automating basic clinical and operational tasks. The seamless integration into medical workflows is a virtue of exemplary embodiments, and exemplary embodiments represents itself as a flexible solution to the last mile problem. As part of monitoring the impact of such an exemplary system on physician behavior and on patients, there should be a level of continuous supervision to capture human-machine interactions as well as mitigate the risk of model drift over time. the implementation of such a system is discussed in section 3.8 herein.

Exemplary use of a smaller encoder language model trained on highly tailored data can demonstrate the potential for this approach to transform hospital operations and the practice of healthcare, and also represents a marked departure from the current trends in language model research that focus on massive, generative models pretrained on large, nonspecific datasets. Nonetheless, even relatively small LLMs may require a substantial amount of compute for pretraining. The exemplary pretraining utilized 24× NVIDIA A100 GPUs for 3 weeks, and exemplary finetuning used 8× A100 GPUs for 6 hours per run. This amount of compute is not commonly accessible to research groups. Exemplary results indicate that massive pretraining may not be necessary for obtaining highly performant models.

Exemplary results also illustrate that high quality datasets for fine-tuning are more valuable than pre-training, and based on the experimental results it may be recommend that users locally finetune an externally pretrained language model when compute is limited. Regarding the choice for the externally pretrained model, it may further be recommend using a model pretrained with a large amount of in-domain clinical text, although note that large, out-of-domain models can be highly performant particularly when combined with in-domain finetuning. Exemplary approach using smaller (<1 billion parameter) LLMs fine-tuned on high quality datasets is markedly different from current trends towards larger (>1 billion parameter) LLMs trained on large, general datasets. Exemplary work with larger, decoder based architectures has also demonstrated a benefit with fine-tuning on medical data or prompt tuning with chain-of-thought, instructions, and related techniques (see, e.g., Refs. [23] and [24]), which further emphasizes the necessity of accounting for the domain shift from general to medical text for some LLM work in the medical sciences.

Physicians are eager to have AI assistants observing care along with them and chiming in with predictions and advice. To take a step towards this vision, exemplary embodiments trained an LLM, NYUTron, on the entire EHR of a large healthcare system to read physician notes and make several of these predictions across a wide range of clinical and operational tasks. Exemplary embodiments deployed NYUTron in a live healthcare environment and demonstrated its efficacy at predicting 30-day readmissions while being integrated seamlessly into clinical workflows.

### 2 Exemplary Methods

### 2.1 Exemplary Dataset

For more detailed dataset statistics and pretraining corpora for other LLMs, see Extended Data Table 6, Table 7.

### 2.1.1 Exemplary Pretraining Dataset

The exemplary dataset included unlabeled clinical notes directly from the NYU Langone EHR. The dataset contains 387,144 patients, 7,247,694 notes, and 4,112,249,482 words in total. NYU Notes were built as follows: Structured Query Language (SQL) scripts were written to query NYU Langone EHR. The queries were prototyped with an interactive web-based editor (Cloudera Hue), then the query downloaded results as comma separated files (CSVs) to NYU Langone's high-performance computing cluster. Notes signed by medical professionals were included (physicians, residents, physician assistants, nurse practitioners, fellows) at Tisch Hospital, NYU Langone Hospital—Brooklyn, NYU Langone Hospital—Long Island, and NYU Langone Orthopedic Hospital from 2011 to 2020 (inclusive). Any notes derived from billing, were labelled as invalid, or empty. The notes were split into 3 sets: training, validation, and test set, with the ratio of 949:50:1. Further, tokens were masked out with 15% probability to create masked text and labels.

NYU Notes—Manhattan: This exemplary dataset was generated from unlabeled clinical notes as the subset of the NYU Notes that are written in Tisch Hospital in Manhattan. The dataset contains 256,217 patients, 4,342,602 notes, 2,381,466,993 words in total.

NYU Notes—Brooklyn: This exemplary dataset was generated from unlabeled clinical notes as the subset of the NYU Notes that are written in NYU Langone Health—Brooklyn. The dataset contains 104,521 patients, 1,337,352 notes, 1,102,078,012 words in total.

### 2.1.2 Exemplary Finetuning Dataset

NYU Readmission: This exemplary dataset was generated from labelled discharge notes (with binary labels for readmission) from the NYU Langone EHR. Most of the notes from this exemplary dataset are a subset of the NYU Notes, with additional discharge notes from 2021 for the temporal test. The dataset contains 413,845 patients, 506,740 notes and 487,395,462 words in total. This exemplary dataset was generated as follows: for each encounter that ended from January 2011 to November 2021, its discharge note was included with a binary label for 30-day all-cause readmission. The “readmitted” label was assigned if the patient has an admission note within 30 days of being discharged. To focus on modelling acute care readmission, discharge notes were excluded from the rehabilitation, dialysis, and palliative care departments because these are not acute care admissions. The dataset was split into 4 sets: training, validation, test, and temporal test set. The first 3 sets are notes from January 2011 to May 2021, with a ratio of 8:1:1. The temporal test set are notes from June to December of 2021. Section 6b herein discusses a visualization of the 4-way split.

NYU Readmission—Manhattan: This exemplary dataset was generated from unlabeled clinical notes as the subset of the NYU Readmission that are written in Tisch Hospital in Manhattan. The dataset contains 240,824 patients, 296,519 notes and 253,622,053 words.

NYU Readmission—Brooklyn: This exemplary dataset was generated from unlabeled clinical notes as the subset of the NYU Readmission that are written in NYU Langone Health—Brooklyn. The dataset contains 94,653 patients, 113,275 notes and 142,767,957 words.

NYU Mortality: This exemplary dataset was generated from history and physical (H&P) note with binary labels for in-hospital mortality from NYU Langone EHR. Most of the notes from this exemplary dataset are a subset of the NYU Notes, with additional H&P notes from 2021 for the temporal test. The dataset contains 371,922 patients, 469,162 notes and 484,467,141 words in total. This exemplary dataset was generated as follows: for each encounter that ended from January 2011 to November 2021, we include its H&P note with a binary label for in-hospital mortality. The positive label was assigned if the patient's discharge disposition is “expired”. The dataset was split into 4 sets: training, validation, test and temporal test set. The first 3 sets are notes from January 2011 to May 2021, with a ratio of 8:1:1, the temporal test are notes from June to December of 2021.

NYU Binned Comorbidity: This exemplary dataset was generated from history and physical (H&P) note with 5-class labels for hospital length of stay (LOS) from NYU Langone EHR. Most of the notes from this exemplary dataset are a subset of the NYU Notes, with additional H&P notes from 2021 for the temporal test. The dataset contains 327,039 patients, 403,579 notes and 422,485,417 words in total. The dataset contains fewer labelled encounters than NYU Mortality and NYU Binned LOS because 22% of the encounters have no ICD codes for calculating Charlson comorbidity index. This missingness motivates the task of predicting binned Charlson comorbidity index with the lack of structured ICD codes.

This exemplary dataset was generated as follows: for each encounter that ended from January 2011 to November 2021, its H&P note was included with a 5-class label for binned Charlson comorbidity index. To generate the labels, one first calculates comorbidity index using the ICD codes and the scoring function in [26]. Then the score was discretized into 5 classes: label 0 was assigned for comorbidity index less than 50% quantile (0), label 1 was assigned for comorbidity index between 50% and 75% quantile (1-2), label 2 was assigned for comorbidity index between 75% and 90% quantile (3-4 days), label 3 was assigned for comorbidity index between 90% quantile and 99% quantile (4-7), and label 4 was assigned for comorbidity index greater than 99% quantile (>7). The dataset was split into 4 sets: training, validation, test and temporal test set. The first 3 sets are notes from January 2011 to May 2021, with a ratio of 8:1:1, the temporal test are notes from June to December of 2021.

NYU Binned LOS: this exemplary dataset was generated from history and physical (H&P) note with quantile labels for hospital length of stay (LOS) from NYU Langone EHR. Most of the notes from this exemplary dataset are a subset of the NYU Notes, with additional H&P notes from 2021 for the temporal test. The dataset contains 371,922 patients, 469,162 notes and 484,467,141 words in total. This exemplary dataset was generated as follows: for each encounter that ended from January 2011 to November 2021, its H&P note was included with a binary label and a quantile label for LOS. For the quantile label, label 0 was assigned for LOS less than 25% quantile (0-2 days), label 1 was assigned for LOS between 25% and 50% quantile (3 days), label 2 was assigned for LOS between 50% and 75% quantile (4-5 days), and label 3 was assigned for LOS greater than 75% quantile (>5 days). The dataset was split into 4 sets: training, validation, test and temporal test set. The first 3 sets are notes from January 2011 to May 2021, with a ratio of 8:1:1, the temporal test are notes from June to December of 2021.

NYU Insurance Denial: This exemplary dataset was generated from history and physical (H&P) notes with binary label for whether the patient's insurance claim is initially rejected, or the claim is directly approved. The dataset contains 54,563 patients, 55,791 notes and 51,270,256 words in total. This exemplary dataset was generated as follows: for each encounter that occurred from May 1, 2021 to Apr. 30, 2022, its H&P note was included with a binary label for insurance denial. A positive label was assigned if the patient's insurance claim status is “final adverse determination” (claim was rejected by insurance and was again rejected upon appeal), or “final-favorable determination” (claim was rejected by insurance and approved upon appeal). The dataset was split into 4 sets: training, validation, test, and temporal test set. The first 3 sets are notes from May 1, 2021 to Feb. 30, 2022, with a ratio of 18:1:1. The temporal test set are notes from Mar. 1, 2022 to Apr. 30, 2022.

NYU Insurance Denial—D/C Notes: This exemplary dataset was generated from discharge (D/C) notes with binary label for whether the patient's insurance claim is initially rejected, or the claim is directly approved. The dataset contains 54,563 patients, 55,791 notes and 49,405,133 words in total. This exemplary dataset was generated as follows: for each encounter that occurred from May 1, 2021 to Apr. 30, 2022, we include its D/C note with a binary label for insurance denial. The label assignment and 4-way split is the same as NYU Insurance Denial dataset.

NYU Insurance Eventual Denial—H&P: This exemplary dataset contains the same notes as NYU Insurance Denial, but the labels are different. The binary label indicates whether the patient's insurance claim is eventually rejected (even after appeal), or the claim is eventually approved (direct approval or approval after appeal).

NYU Insurance Eventual Denial—D&C: This exemplary dataset contains the same notes as NYU Insurance Denial—D&C, but the labels are different. The binary label indicates whether the patient's insurance claim is eventually rejected (even after appeal), or the claim is eventually approved (direct approval or approval after appeal).

i2b2-2012 NER: This is an open dataset released by the Harvard Medical School as part of an annual clinical NLP challenge (see, e.g., Ref. [27]). This exemplary dataset is a well-known benchmark in the clinical NLP community. The task is to identify and classify clinical concepts (e.g., treatments), clinical departments (e.g., surgery), occurrences of events (e.g., admission) and evidentials (e.g., the patient complained) from de-identified clinical notes from Boston's Beth Israel Hospital. The dataset contains no more than 310 patients, 310 notes and 636,000 words. We downloaded the dataset as a compressed tar.gz file from n2c2 data portal after our use application is approved.

MIMIC-III (see, e.g., Ref. [28]) Readmission: This is an open dataset of ICU EHR released by MIT and Boston Beth-Israel Medical Center. A set of 52,726 discharge notes were collected and a 30-day all-cause readmission label was created by checking any subsequent encounter within 30 days. The readmission rate is 6%. The data was split into train-val-test set in a 8:1:1 ratio.

### 2.1.3 Exemplary Deployment Dataset

NYU Readmission—Deployment: This exemplary dataset includes discharge note with binary labels for readmission from our deployment engine and Langone EHR. From January to April 2022, every time a discharge note is signed by a physician, the note is sent to our custom inference engine for NYUTron's prediction. A pair of discharge note and prediction is recorded in a database. The database contained 27,376 patients, 29,287 notes and 34,669,963 words by the end of the study period.

### 2.1.4 Exemplary Structured Dataset

NYU Readmission—LACE: This exemplary dataset was generated from structured LACE [29] features with binary labels for readmission for comparison against the unstructured models. The dataset contains structured features for all encounters in NYU Readmission. LACE is a traditional clinical prediction rule for readmission with 4 features: Length of stay, Acuity of readmission, Comorbidity index, and number of recent Emergency department visit. The dataset was generated as follows: for every encounter in the NYU Readmission dataset, the 4 LACE features were collected from the NYU Langone EHR. The length of stay is the difference (in days) between the discharge date and the admission date. The acuity of readmission is a binary feature for whether the patient was admitted to the emergency department. The comorbidity index is calculated with the ICD-9 or ICD-10 codes for chronic diseases, based on the mapping procedure described in Ref. [30] and the scoring function described in Ref. [26]. The number of emergency department visits is calculated from the patient's encounter history up to 6 months before the admission date.

NYU Readmission—LACE, Manhattan: This exemplary dataset was generated from structured LACE features as the subset of the NYU Readmission—LACE that are written in Tisch Hospital in Manhattan.

NYU Readmission—LACE, Brooklyn: This exemplary dataset was generated from structured LACE features as the subset of the NYU Readmission—LACE that are written in NYU Langone Health—Brooklyn.

NYU Mortality—SAPS2+APACHE2: This exemplary dataset was generated from structured “SAPS2+APACHE2” features with binary labels for in-hospital mortality in order to compare against the unstructured data. The dataset contains a subset of structured “SAPS2+APACHE2” features for all encounters in NYU Mortality. “SAPS2+APACHE2” features are a subset of features used in SAPS2 model (see, e.g., Ref. [15]) and APACHE2 model (see, e.g., Ref. [16]) for ICU Mortality prediction. The subset of features that are available in Langone EHR were selected. The following 12 features were included: age (numerical), mean heart rate (numerical), systolic blood pressure (numerical), atrial temperature (numerical), blood urea nitrogen (numerical), sodium (numerical), potassium (numerical), bilirubin (numerical), white blood cell count (numerical), ph (numerical), creatine (numerical), hematocrit (numerical). Additionally, 1 feature was added: department specialty (categorical). The following features were excluded due to unavailability: PaO2/FiO2 (ratio of arterial oxygen partial pressure to fractional inspired oxygen), whether patient is on mechanical ventilation or CPAP (continuous positive airway pressure), bicarbonate, urine output, GCS (Glas-glow Coma Scale), presence of metastatic cancer or hematologic malignancy or AIDs, whether admission is scheduled.

NYU Binned LOS—Lisbon Portugal: This exemplary dataset was generated from structured “Lisbon Portugal” features with binary labels for in-hospital mortality in order to compare against the unstructured data. The dataset contains a subset of features used in “Lisbon Portugal” dataset (see, e.g., Ref. [18]) (which is widely used in the LOS prediction literature) for all encounters in NYU Binned LOS. A subset of 12 features that are available in Langone her were selected: gender (categorical), age as measured by the difference in years between birth date and the admission date (numerical), highest level of education (categorical), country (categorical), postal code as address (categorical), marital status (categorical), admission type (categorical), admission service type (categorical), provider id (categorical), department specialty (categorical), procedure name (categorical), number of previous admission (numerical). Diagnosis was left out because it is not always available at the time of writing history and physical notes. The following 3 features were excluded due to difficulty of finding it in Langone EHR: GDH (homogeneous group diagnosis code), GCD (great diagnostic category), treatment.

NYU Insurance Denial—Claim forms: This structured exemplary dataset was generated based on NYU Insurance Denial for comparison against the unstructured data model. The dataset contains structured features for all encounters in NYU Insurance Denial and has the same splits as NYU Insurance Denial. The selection of structured features is based on the features in [19], which builds a model that predicts insurance claim denial from demographic and care-related features found in the claim form. 8 available features in Langone her were found: patient name (categorical), age (numerical), gender (categorical), postal code as a generalization of address (categorical), insurance brand (categorical), first insurance plan name (categorical), provider id (categorical), provider type (categorical). Additionally, 4 features were added based on clinician's inputs: second insurance plan code (categorical), a binary flag for surgical case (categorical), a binary flag for emergency department cases (categorical), a binary flag for Medicare Fee-for-Service users (categorical). 6 features were left out (see, e.g., Ref. [19]) due to difficulty of search: patient's relationship to the insured, network type, whether the claim is a resubmission, diagnosis pointer, charges of service, and prior authorization number.

### 2.2 Exemplary Preprocessing

Pretrain Dataset (NYU Notes, NYU Notes—Manhattan, NYU Notes—Brooklyn): Using these exemplary datasets, it is possible to train an uncased BERT wordpiece tokenizer with a vocab size of 50,000 tokens, a maximum sequence length of 512 tokens, and special tokens [SEP], [PAD], [UNK], [MASK], and [CLS]. Since most of the clinical notes have more than 512 tokens, it is possible to split every long note into non-overlapping chunks that are under the maximum sequence length. Specifically, it is possible to split each note into sentences using spaCy (see, e.g., Ref. [31]) en core web sm and tokenize each sentence. For sentences that are longer than 512 tokens, it is possible to truncate it. Next, for all the tokenized sentence in the same note, it is possible to concatenate them into groups such that each group has exactly the maximum sequence length. It is possible to discard any remainder group (with length strictly less than the maximum) of a long note.

Finetune Dataset (NYU Readmission, NYU Readmission—Manhattan, NYU Readmission—Brooklyn, NYU Mortality, NYU Binned LOS, NYU Insurance Denial, NYU Binned Comorbidity): Using the tokenizer trained with NYU Notes, it is possible to first tokenize the discharge note. It is possible to truncate notes that exceed the maximum sequence length of 512 tokens. It is possible to leave for the future to design a language model that efficiently reads longer clinical notes (See supplementary 7b for the impact of note lengths on language model's performance.) i2b2-2012 NER: it is possible to first decompress the tar.gz files into folders of xml files. Then, it is possible to convert the xml files to brat format. Next it is possible to convert brat files to bio files. Finally, it is possible to write a custom HuggingFace (see, e.g., Ref. [32]) dataloader to convert the folder of bio files into a HuggingFace dataset. The exemplary code for preprocessing is available at Github.

Deployment Dataset: The notes were first cleaned by stripping out html artifacts. Then it is possible to tokenize the discharge note using NYUTron's tokenizer. It is possible to truncate notes that exceed the maximum sequence length of 512 tokens.

Structured Dataset (NYU Readmission—LACE, NYU Mortality—SAPS2+APACHE2, NYU Binned LOS—Lisbon Portugal, NYU Insurance Denial—Claim forms): When there is a missing numerical feature (e.g., the average heart rate is NaN), it is possible to fill in the feature as the average feature across the train set. For missing categorical features (the admitting department is “unspecified”), it is possible to leave it as category “None”.

### 2.3 Exemplary Pretraining

An exemplary pretrain can include a 109-million parameter BERT model using preprocessed NYU notes and the masked language modeling (MLM) objective for 3 week (96 epochs) on 24× NVIDIA A100 GPUs distributed over 3 compute nodes until the validation loss starts to plateau. The model has 12 hidden layers with dimension 768, 12 attention heads per layer. It is possible to use a per-device training batch size of 64, and saved every 2000 steps. We use Zero Redundancy AdamW optimizer with a constant learning rate of 5·10-5, FP16 mixed precision, and stage-2 parallelization (see, e.g., Refs. [33] and [34]).

### 2.4 Exemplary Finetuning

NYUTron+Discharge Notes for Readmission Prediction: It is possible to replace the trained MLM classifier with a randomly initialized linear classifier after the last hidden layer of the pretrained BERT model. It is possible to finetune the model end-to-end using the training set of the NYU Readmission dataset for 10 epochs, evaluating the validation AUC every half epoch and early stopping with a patience of 5. It is possible to use the following hyper-parameters from manual tuning based on validation AUC: a learning rate of 2·10-5, a weight decay of 0.01, and a per-device batch size of 4. It is possible to optimized the cross entropy loss using Adam optimizer (see, e.g., [35]). While varying the size of the dataset (N∈{102, 103, 104, 105, 3.92336·105}), it is possible to finetune the pretrained model using subsamples of the NYU Readmission dataset and evaluate their AUC on the temporal test set. For each size of subsamples, we run 5 experiments with distinct random seeds (0, 13, 24, 36, 42). For comparison, it is possible to look at the median AUC and the standard deviation of the 5 experiments.

NYUTron+H&P Notes for In-hospital Mortality Prediction: It is possible to replace the trained MLM classifier with a randomly initialized linear classifier after the last hidden layer of the pretrained BERT model. It is possible to finetune the model end-to-end using the training set of the NYU Mortality dataset for 10 epochs, evaluating the validation AUC every half epoch and early stopping with a patience of 5. It is possible to use the following hyper-parameters from manual tuning based on validation AUC: a learning rate of 2·10-5, a weight decay of 0.01, and a per-device batch size of 4. It is possible to optimize the cross entropy loss using Adam optimizer [35]. Using the full dataset, it is possible to finetune the pretrained model using subsamples of the NYU Mortality dataset and evaluate their AUC on the temporal test set. For each size of subsamples, it is possible to perform 5 experiments with distinct random seeds (0, 13, 24, 36, 42). For comparison it is possible to review the median AUC and the standard deviation of the 5 experiments.

NYUTron+H&P Notes for Binned Comorbidity Prediction: It is possible to replace the trained MLM classifier with a randomly initialized linear classifier after the last hidden layer of the pretrained BERT model. It is possible to finetune the model end-to-end using the training set of the NYU Binned Comorbidity dataset for 10 epochs, evaluating the validation OVR (one-versus rest) AUC every half epoch and early stopping with a patience of 5. It is possible to use the following hyper-parameters from manual tuning based on validation OVR (one-versus rest) AUC: a learning rate of 2·10-5, a weight decay of 0.01, and a per-device batch size of 4. It is possible to optimize the cross entropy loss using Adam optimizer (see, e.g., [35]). Using the full dataset, it is possible to finetune the pretrained model using subsamples of the NYU Binned Comorbidity dataset and evaluate their OVR AUC on the temporal test set. For each size of subsamples, it is possible to run 5 experiments with distinct random seeds (0, 13, 24, 36, 42). For comparison it is possible to look at the median OVR AUC and the standard deviation of the 5 experiments.

NYUTron+H&P Notes for Binned LOS Prediction: It is possible to replace the trained MLM classifier with a randomly initialized linear classifier after the last hidden layer of the pretrained BERT model. It is possible to finetune the model end-to-end using the training set of the NYU Binned LOS dataset for 10 epochs, evaluating the validation AUC every half epoch and early stopping with a patience of 5. It is possible to use the following hyper-parameters from manual tuning based on validation OVR AUC: a learning rate of 2·10-5, a weight decay of 0.01, and a per-device batch size of 4. It is possible to optimize the cross entropy loss using Adam optimizer (see, e.g., [35]). Using the full dataset, it is possible to finetune the pretrained model using subsamples of the NYU Binned LOS dataset and evaluate their AUC on the temporal test set. For each size of subsamples, it is possible to run 5 experiments with distinct random seeds (0, 13, 24, 36, 42). For inference it is possible to combine the last 2 classes: label 3 (quantile 90-99%) and label 4 (quantile 99%+) because label 4 is very sparse. For comparison it is possible to look at the median OVR AUC and the standard deviation of the 5 experiments.

NYUTron+H&P Notes for Insurance Denial Prediction: It is possible to replace the trained MLM classifier with a randomly initialized linear classifier after the last hidden layer of the pretrained BERT model. It is possible to finetune the model end-to-end using the training set of the NYU Insurance Denial dataset for 10 epochs, evaluating the validation AUC every half epoch and early stop-ping with a patience of 5. It is possible to use the following hyper-parameters from manual tuning based on validation AUC: a learning rate of 2·10-5, a weight decay of 0.01, and a per-device batch size of 4. It is possible to optimize the cross entropy loss using Adam optimizer (see, e.g., Ref. [35]). Using the full dataset, it is possible to finetune the pretrained model using subsamples of the NYU Insurance Denial dataset and evaluate their AUC on the temporal test set. For each size of subsamples, it is possible to run 5 experiments with distinct random seeds (0, 13, 24, 36, 42). For comparison it is possible to look at the median AUC and the standard deviation of the 5 experiments.

NYUTron+Clinical Notes for Named Entity Recognition: It is possible to perform the finetuning experiments as follows: For each LLM in Extended Data Table 6, it is possible to initialize a HuggingFace token classification model with the LLM as the pretrained checkpoint. It is possible to finetune the model using i2b2-2012 NER for 10 epoch using AdamW optimizer [34] with a learning rate of 2·10-5, a weight decay of 0.01, a batch size of 4, evaluating every 50 steps, and early stopping based on roc auc with a patience of 1. It takes 20 to 40 minutes on 1 node of 4 NVIDIA 17-GiB V100 GPUs. It is possible to perform finetuning 5 times with random seeds 0, 13, 24, 36, 42 and record the average and the standard deviation of micro-averaged f1 score (excluding the label for non-entity: ‘O’).

NYUTron+MIMIC-III Readmission: It is possible to perform the finetuning experiments as follows: For both NYUTron and BioClinicalBert, it is possible to initialize a HuggingFace token classification model with the LLM as the pretrained checkpoint. It is possible to finetune the model using MIMIC-III Readmission for 10 epoch using AdamW optimizer [34] with a learning rate of 2·10-5, a weight decay of 0.01, a batch size of 16, evaluating every half epoch. It is possible to perform finetuning 5 times with random seeds 0, 13, 24, 36, 42.

### 2.5 Exemplary Deployment

The finetuned model is converted to a high performance format (Onnx or TensorRT), and loaded into our deployment platform: an NVIDIA Triton inference engine which interfaces with the Langone EHR via the HLA7 FHIR [36] interface. For our consideration of performance, security, reliability and interpretability, as further discussed in section 3.8 herein.

Exemplary deployment platform can include a modified version of NVIDIA's Triton Inference Server we named NYUTriton (pronounced “nutrition” because it is good for the health system). NVIDIA Triton supports GPU-, x86-, and ARM® CPU-based inferencing and several key features including dynamic batching, concurrent execution, a highly flexible model specification interface, and the ability to support a wide range of deep learning frameworks and accelerated model formats for maximal throughput. It is possible to modify NVIDIA Triton to inter-face seamlessly with HuggingFace formatted language models so as to provide a uniform and highly flexible crossover point between our development and production pipelines. Trained models are saved in a standard HuggingFace-style format, and then converted into Onnx, and then TensorRT to obtain sub-millisecond scale inference results. NYUTriton is hosted on a dedicated inference server which consists of a AMD Threadripper 3960X (24 cores, 3.8 GHz), 2× RTX 3090 GPUs, and 128 Gb of DDR5 system memory purchased from Lambda Labs.

Upon the signing of discharge summaries in EPIC, the HL7 FHIR interface connects with NYUTriton and sends a JSON payload consisting of the dis-charge summary and metadata specifying the underlying readmission model and sender. NYUTriton preprocesses the text, runs an inference job with the accelerated NYUTron readmission model, and returns the model's inference result to a secondary orchestration server which writes the result to a database and generates an e-mail to the sending physician.

### 2.6 Exemplary Structured Baselines

The structured baselines are: (1) SAPS2/APACHE2 features+XGBoost for In-hospital Mortality Prediction, (2) LACE features+XGBoost for Read-mission Prediction, (3) Lisbon-Portugal features+XGBoost for Binned LOS Prediction, (4) Claim forms features+XGBoost for Insurance Denial Prediction.

For all structured baselines, it is possible to use the xgboost library to train an extreme gradient boosted tree classifier with a binary logistic loss (multi-class softmax loss for more than 2 class). It is possible to use scikit-learn's randomized search to search hyperparameters among minimum child weight from {1, 5, 10}, gamma from {0.5, 1, 1.5, 2, 5}, subsample from {0.6, 0.8, 1}, col-sample bytree from {0.6, 0.8, 1.0, max depth from {3, 4, 5}, learning rates from {0.001, 0.01, 0.1, 0.5}, n estimators from {10, 100, 1000} for 100 iterations based on auroc score (ovr-auroc score for multiclass) based on 3-fold cross validation [37]. It is possible to run each experiment 5 times with distinct random seeds (0, 13, 24, 36, 42). For mortality, binned comorbidity, binned LOS, insurance denial, it is possible to ran the experiment with the full dataset. For readmission, it is possible to train the model using subsamples (N∈{102, 103, 104, 105, 3.92336·105}) of the NYU Readmission—LACE dataset.

### 2.7 Exemplary Metrics

It is possible to evaluate the five tasks (In-hospital mortality prediction, binned comorbidity index prediction, 30-day all-cause readmission prediction, binned LOS prediction, insurance denial prediction) with AUC for binary classes and One-versus-Rest (OVR) AUC for multiclass. Area under the receiver operating curve (AUC) is the area under the 2-dimensional curve consisting of tuples of the form (tpr,fpr) resulted from different decision thresholds.

It is possible to additionally evaluate readmission prediction with the following metrics: true positive rate (TPR), false positive rate (FPR), precision, recall, and f1 score, all of which have range in [0, 1].
True positive rate is the ratio between the number of correctly predicted readmissions and the number of positive labels.
False positive rate is the ratio between the number of falsely predicted readmission and the number of negative labels.
Precision is the ratio between the number of correctly predicted readmissions and the number of cases predicted to be readmitted.
Recall is same as the true positive rate, or the ratio between the number of correctly predicted readmissions and the number of positive labels.
F1 scores is the ratio between the product of precision and recall and the sum of precision and recall.

It is possible to evaluate named entity recognition using micro-averaged NER-f1 score. The NER-f1 score is similar to normal f1 score, except that the non-entity label “O” is excluded for calculation.

### 2.8 Exemplary Detailed Evaluation of Readmission Prediction

### 2.8.1 Exemplary Baseline Algorithms for Retrospective Study

It is possible to compare NYUTron against physicians. The work can be compared with 6 physicians with different levels of seniority: 3 attending physicians, and 3 residents. The physicians were asked to review discharge summaries and predict whether or not the described patient would come back to the hospital within 30 days.

NYUTron can be compared against four other LLMs and two machine learning models.
1. “random-init” is a BERT-base-uncased model with randomly initialized parameters.
2. “web-wiki”, is a BERT-base uncased model that is pretrained using web texts (from BookCorpus dataset [38]) and Wikipedia articles (from English Wikipedia dataset [39]).
3. “web-wiki+bio”, is a BERT model pretrained using web texts, Wikipedia articles, pubmed abstracts [40] and PMC full articles [41]
4. “web-wiki+bio+clinical”, or gatortron-og [42], is a Megatron-BERT [43] model pretrained using web texts, Wikipedia articles, Pubmed abstracts, PMC full articles, MIMIC-III [28] notes, and de-identified clinical notes from the University of Florida Health.
5. lace+xgb reads structured LACE features (from traditional clinical prediction rule) with an extreme gradient boosted tree model [14].
6. tf-idf+xgb reads corpus-level bag-of-words features with an extreme gradient boosted tree model.

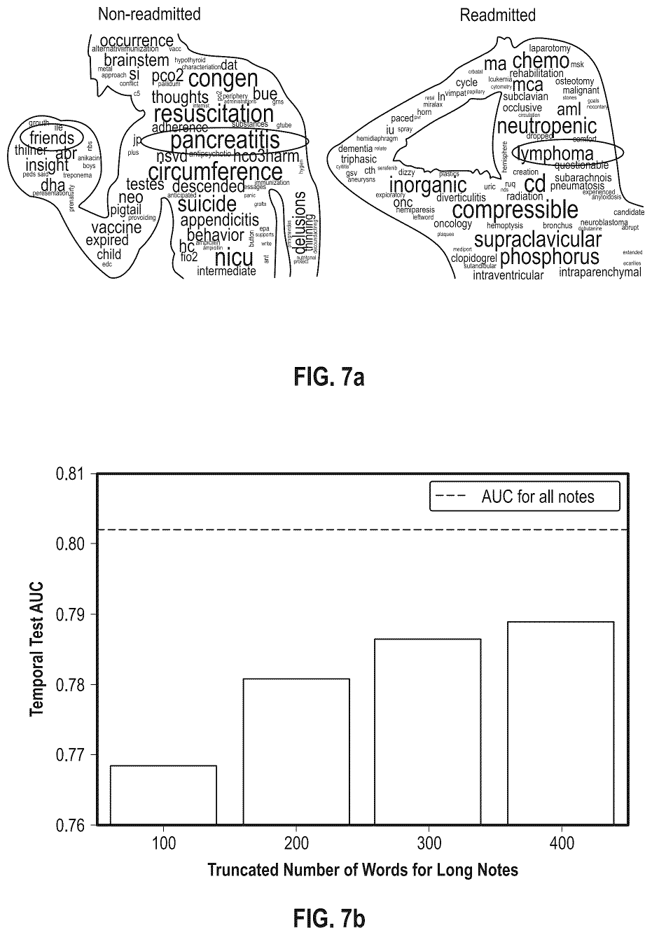

Detailed statistics and examples of the pretraining corpora are shown in FIGS. 7(a), 7(b) and 6(a).

For example, FIGS. 7(a) and 7(b) provide exemplary illustrations of an exemplary readmission wordcloud and the impact of note length on readmission prediction according to exemplary embodiments. FIG. 7(a) shows an illustration of an exemplary word cloud of discharge notes from non-readmitted patients (left) and readmitted patients (right) according to exemplary embodiments. The word clouds were constructed based on non-readmitted and readmitted labels from NYUTron where a word with a larger log odds ratio has a larger font size. Non-readmitted patients seem to have milder diseases such as “pancreatitis” and have “friends” who can pick them up upon discharge. The readmitted patients have more serious disease such as “lymphoma”, which requires frequent hospital visits for chemotherapy and radiotherapy.

FIG. 7(b) shows an illustration that an exemplary NYUTron performance increases with more complete input notes. To attempt to estimate performance as a function of sequence length, a subset of “long notes” was sampled from the temporal test set. Each note in this subset has no less than 400 words, or approximately 512 tokens. It is possible to truncate these long notes to 100, 200, 300 and 400 words while keeping their readmission labels fixed in order to demonstrate the incremental gain in performance as it is possible to capture proportionally more information from each of these “long notes”. The dashed line is the AUC of all notes. This figure shows that processing more words from the possible input leads to a better evaluation performance and confirms that there is a clear potential for improving performance by increasing maximum sequence length.

2.8.2 Exemplary Comparison with Physicians

It is possible to randomly sample 20 discharge notes from the random test set and ask 6 doctors with different seniority to predict whether the patient would come back within 30 days. The 6 physicians include 3 attending neurosurgeon, 2 neurosurgery residents, and 1 ICU resident.

It is also possible to use REDCap to perform the survey and gave physicians unlimited time. The survey is structured as follows: for each case, we ask “will this per-son be admitted within 30 days?”, followed by the discharge summary. The physician can choose to answer “Yes” or “No”. If the patient truly came back within 30 days, it is possible to have/provide 3 follow-up questions to assess the characteristics of the subsequent readmission. First, it is possible to ask “is this readmission related to the prior discharge?”, followed by the history and physical note of the subsequent readmission. The physician can answer “Yes”, “No”, “Partial” or “Does not meet medicare criteria for 30 d readmission”. The second follow-up question can be “Is this readmission preventable?”, to which the physician can answer “Yes”, “No” or “Partial”. The third follow-up question is a free response: “Any comments?”, where the physicians can explain why the readmission is partially related to prior discharge, or why the readmission is partially preventable.

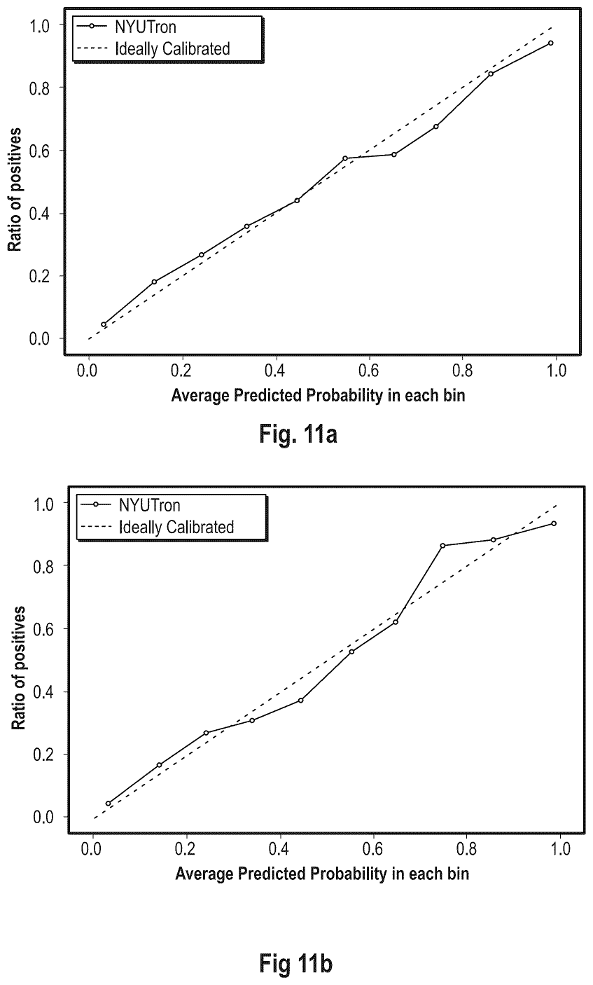

To collect NYUTron's predictions, it is possible to use the text classification pipeline from HuggingFace to perform inference on the 20 discharge notes. For each discharge note, the pipeline outputs a predicted probability for readmission. It is possible to convert this predicted probability to a binary label with a threshold of 0.07 (a predicted probability no less than 0.07 is converted to a positive label). It is possible to choose 0.07 as the decision boundary, because it is the minimum threshold that gives us above 80% validation recall among the thresholds {0.01·n: n∈{1, . . . , 90}}(the 80% criteria is chosen based on clinical applicability). See Extended Data FIG. 11 for NYUTron's calibration curve.

FIGS. 11(a)-11(b) shows exemplary graphs showing NYUTron's calibration curve for temporal test and prospective deployment according to exemplary embodiments of the present disclosure. As a reference, the orange broken line is the calibration curve of an ideally calibrated classifier. The blue solid line is NYUTron's calibration curve. Overall the model is well calibrated to the 30-day readmission task. FIG. 11(a) shows a calibration curve for temporal test, and FIG. 11(b) shows a calibration curve for prospective test.

2.8.3 Exemplary Comparison with Other Language Models

Discharge Notes+Other LLMs for Readmission Prediction: The exemplary dataset, hyperparameter, evaluation and software libraries for finetuning other LLMs are the same as finetuning NYUTron. The pretrained LLMs are constructed as follows: “random init” is a bert-base-uncased model with reset parameters. “web-wiki” is the bert-base-uncased model. “web-wiki+bio” is the dmis-lab/biobert-base-cased-v1.2 model. “web-wiki+bio+clinical” is Gatortron-og download from nVidia NGC and converted to HuggingFace checkpoint using convert megatron bert checkpoint.

Clinical Notes+Other LLMs for Named Entity Recognition: The exemplary dataset, hyperparameter, evaluation and software libraries for finetuning other LLMs are the same as finetuning NYUTron. The pretrained LLMs are the same as the baseline LLMs for predicting readmission from discharge notes.

2.8.4 Exemplary Comparison with Machine Learning Models

LACE features+XGBoost for Readmission Prediction: Using the NYU Readmission—LACE dataset, it is possible to use the xgboost library to train a extreme gradient boosted tree classifier with a binary logistic loss with hyperparameter search. It is possible to use scikit-learn's randomized search to search among minimum child weight from {1, 5, 10}, gamma from {0.5, 1, 1.5, 2, 5}, subsample from {0.6, 0.8, 1}, colsample bytree from {0.6, 0.8, 1.0, max depth from {3, 4, 5}, learning rates from {0.001, 0.01, 0.1, 0.5}, n estimators from {10, 100, 1000} for 100 iterations based on auroc score on the validation set [37]. It is possible to train the model using subsamples (N∈{102, 103, 104, 105, 3.92336·105}) of the NYU Readmission—LACE dataset and evaluate their AUROC on the temporal test set. For each size of subsamples, it is possible to run 5 experiments with distinct random seeds (0, 13, 24, 36, 42). For comparison it is possible to look at the median AUROC and the standard deviation of the 5 experiments.

XGBoost+TF-IDF for Readmission Prediction: It is possible to transform the texts from the NYU Readmission dataset into tf-idf (term frequency—inverse document frequency) embeddings and use a xgboost classifier with binary logistic loss to predict readmission. It is possible to use raytune (see, e.g., Ref. [44]) to search hyperparameters among a maximum tf-idf features from {512, 5000}, a max depth from a quantized random integer from 3 to 16 with an interval of 4, learning rate from a log uniform distribution from 10-2 to 10-1, gamma from a quantized uniform distribution from 0 to 12 with an interval of 4, min child weight from a quantized uniform distribution from 0 to 8 with an interval of 4, reg lambda from a quantized uniform distribution from 0 to 10 with an interval of 2, colsample bytree from a uniform distribution from 0.7 to 1, scale pos weight from a quantized uniform distribution from 0 to 50 with an interval of 10, n estimator from a quantized integer distribution from 50 to 300 with an interval of 50. It is possible to train the model using subsamples (N∈{102, 103, 104, 105, 3.92336·105}) of the NYU Readmission dataset and evaluate their AUROC on the temporal test set. For each size of subsamples, it is possible to run 5 experiments with distinct random seeds (0, 13, 24, 36, 42). For comparison we look at the median AUROC and the standard deviation of the 5 experiments.

### 2.8.5 Exemplary Comparison of Multi-Site Pretraining-Finetuning

It is possible to compare NYUTron with its 4 variants (pretrained and finetuned using data from different sites).
NYU Notes—Manhattan+NYU Readmission—Manhattan
NYU Notes—Manhattan+NYU Readmission—Brooklyn
NYU Notes—Brooklyn+NYU Readmission—Brooklyn
NYU Notes—Brooklyn+NYU Readmission—Manhattan

The hyperparameter, evaluation and software libraries for finetuning NYUTron variants are the same as finetuning NYUTron.

### 2.8.6 Exemplary Analysis of Prospective Performance

Based on the temporal test performance in the retrospective study, it is possible to selected a finetuned model with a decision threshold of 0.07 for use in the prospective trial.

Comparison of mortality rate and length of stay: To assess the condition of the readmitted patients who were correctly predicted (N=3, 298), it is possible to compare their in-hospital mortality rate and length of hospitalization with patients who were admitted in the same period. It is possible to collect patients who were admitted from February to May of 2022 (N=30, 548) and compare their in-hospital mortality rate and length of stay with the readmitted patients caught by NYUTron from January to April of 2022. It is possible to use two sided Welch's t-test (with the null hypothesis that the two groups have the same average) to check the statistical significance of our comparison [45].

Assessing NYUTron's clinical impacts with physician reviews: a post-hoc analysis of re-admitted patients can be performed in the prospective cohort to better understand model performance in a real world environment and in anticipation of creating targeted interventions based on model outputs. One hundred readmitted patients were sampled from the five largest departments at Langone by patient volume: Internal Medicine, Pediatrics, General Surgery, Obstetrics and Gynecology, and Hematology and Oncology. Each department contributed 20 cases, with 10 cases having the highest predicted probabilities in that department, and 10 cases with the lowest predicted probabilities. All cases had their EncounterID's logged for their index discharge and readmission on a secure online platform. A standardized questionnaire was constructed for manual review asking: whether the readmission was planned, whether the readmission met CMS criteria for a penalized 30-day readmission, whether the readmission was preventable, whether an adverse event occurred on readmission, whether any adverse events were preventable, and whether the reviewing physicians had any comments on the case. A team of 10 physicians from Internal Medicine and Neurosurgery were randomly assigned cases to be reviewed in pairs, with a disagreement between reviewers being adjudicated by a third physician reviewer. For determining whether a readmission is preventable, the reviewer looks at the discharge note of the inference encounter and the H&P note of the readmitted encounter.

### 3 Exemplary Supplementary Discussion

### 3.1 Exemplary Previous Works

Traditional clinical prediction rules that have existed for decades relies on a small set of hand-selected structured features. Three well-known examples are CHADS2 score for atrial fibrillation stroke risk, Child-Pugh score for cirrhosis mortality, and Well's criteria for pulmonary embolism (see e.g., Refs. [1-4]). An example for readmission prediction is the LACE score, which uses 4 features: Length of stay, Acuity of readmission, Comorbidity index and the number of recent visits to the Emergency department.

Approaches that are based on traditional machine learning models learn from a set of automatically selected structured features (see e.g., Refs. [20] and [46]). For example, Duke University Health System use regression with L1 regularization to select features from patient age, diagnosis variables, laboratory variables, medications, order types and utilization variables (see e.g., Ref. [47]). Their readmission pre-diction model is a regression model on the selected features. (See Supplemental 3.5 for a complexity comparison with NYUTron.)

Another approach represents clinical notes with embeddings from traditional NLP models. For example, to predict readmission from discharge notes, e.g., Refs. [48, 49] passes the LDA (Latent Dirichlet allocation)/TF-IDF (Term frequency-inverse document frequency) embeddings of discharge notes to an 2-class SVM (support vector machine).

With the advent of EHR, another approach for a clinical prediction is to apply deep learning to high-dimensional structured EHR data. This disclosure will refer to them as “structured EHR” approach. For example, e.g., Ref. [50] takes in the entire EHR associated to a patient using the FHIR format (with task-specific labels) and train an RNN with end-to-end.

Recently, researchers start to use clinical texts from electronic health record to train large language models. ClinicalBERT pretrained a BERT model using notes from MIMIC-III and finetuned the pretrained model for ICU readmission prediction (see e.g., Ref. [51]). Gatortron pretrained a 345-million parameter Megatron-BERT model using notes from the University of Florida Health and finetuned the language model for 5 clinical NLP tasks including named entity recognition (see e.g., Ref. [42]).

The gap: Traditional clinical prediction rules and traditional machine learning models rely on structured data, which is often missing from hospital EHRs. Traditional NLP models do not benefit from pretraining with an increasing amount of unlabeled clinical notes. Structured EHR approaches also faces issues with missing structured features, not leveraging the vast amount of unlabeled data, and the high cost of implementation. (See Supplemental 3.4 for an example.) While recent studies on clinical language models show potential for translating advances in NLP to improving quality of health-care, they are limited in that (1) they evaluate on a small subset of patient population (e.g., ICU patients from MIMIC-III; patients with strokes) and (2) they did not perform prospective evaluation, which is better resembles the deployment setup by hardening the model and testing it outside the development environment.

The systems, methods, apparatus and computer-accessible medium according to the exemplary embodiments of the present disclosure relates to pretraining a large language model on an entire health system's identified clinical notes and deploy the fine-tuned model for a prospective trial for all patients. The systems, methods, apparatus and computer-accessible medium according to the exemplary embodiments of the present disclosure indicate that the exemplary clinical language model has a wide breadth of applicability to several clinical and operational tasks, as demonstrated by their improved performance over traditional structured data baselines. On a specific clinical predictive task (readmission prediction), the systems, methods, apparatus and computer-accessible medium according to the exemplary embodiments of the present disclosure indicated the benefit of pretraining with clinical texts, the cross-site generalizability through local finetuning, and the deployability with a prospective, non-interventional, single-arm trial.

### 3.2 Exemplary Details on Insurance Denial Prediction

For patients with insurance, hospitals receive compensation from their insurance companies by submitting insurance claims that document the details of the visit such as procedures done and necessity of the procedure. However, the claimed amount does not always get fully reimbursed, which increases the operating costs of a health system.

The task of insurance denial is to predict (at the point of care) whether the claim associated with that visit will be denied by the insurance providers. This would help reduce unnecessary out-of-pocket costs of the patients and the financial stress of the health system.

In the exemplary dataset, it is possible to consider three possible outcomes of a insurance claim: (1) the claim is directly approved, (2) the claim is initially rejected, but approved upon appeal, (3) the claim is initially rejected, and still rejected upon appeal.

It is possible to consider a claim is “initially denied” for outcomes (2) and (3), and a claim is “directly approved” for outcome (1). It is possible to consider a claim is “eventually denied” for outcomes (3), and a claim is “eventually approved” for outcome (1) and (2).

It is possible to perform four types of prediction using the same method described in Method 2.4 “NYUTron+H&P Notes for Insurance Denial Prediction”:
1. Using NYUTron Insurance Denial dataset, predict whether a claim is initially denied from H&P notes (shown in FIG. 2c, AUC 87.2%±0.246%).
2. Using NYUTron Insurance Denial—D/C Notes dataset, predict whether a claim is initially denied (AUC 87.71%±0.188%).
3. Using NYUTron Insurance Eventual Denial—H&P Notes dataset, predict whether a claim is eventually denied (87.54%±0.312% AUC).
4. Using NYUTron Insurance Eventual Denial—D/C Notes dataset, predict whether a claim is eventually denied (AUC 88.0%±0.313%).

### 3.3 Exemplary Details on the Readmission Problems

### 3.3.1 Exemplary Significance of Readmission Prediction

The present disclosure chose, e.g., readmission prediction because it is a classic, well-studied clinical predictive problem with practical clinical significance. Readmission puts patients at risk medically and financially, and reducing readmission rates could improve the quality of care. Every year, 1.15 billion patients are dis-charged globally, and in the United States 14% of discharged patients are ultimately readmitted. Nationally, readmitted patients are, on average, associated with an extra cost to providers of $15,000 (see e.g., Ref. [52]). To reduce preventable readmissions, the Center for Medicare and Medicaid Services (CMS) launched a hospital readmission reduction program that decreases payments to hospitals according to the rate of unplanned 30-day readmission. Due to the significance of this problem both clinically and operationally, several attempts (see e.g., Refs. [47] and [51]) have been made to build and deploy 30-day readmission models by both health systems and EHR vendors with varying results.

It is possible to estimate the scale of readmission prediction problem as follows:
1) To estimate the number of patients discharged annually, it is possible to use the number of hospital discharges per one thousand person in OECD countries in 2017. OECD countries have 17.9% of the world's 7.52 billion populations, and 154 hospital discharges per 1000 population (see e.g., Refs. [53] and [54]). Assuming that the discharge rate is similar in non-OECD countries, it is possible to estimate the total number of hospital discharge in 2017 around the world as 7.52-154≈1.16 billion discharges.
2) To investigate how often discharged patients get readmitted, it is possible to use the readmission rate and cost from United States. In 2018, United States has a 14% readmission rate with an average readmission cost of $15,200 (see e.g., Ref. [52]).

To reduce preventable readmission in United States, Center for Medicare and Medicaid Services (CMS) launched a Hospital Readmission Reduction Program (HRRP). Starting from Oct. 1, 2012, the U.S. government reduces a maximum of 3% of payments to hospitals with excessive readmission, as measured by “30-day risk-standardized unplanned readmission” (see e.g., Ref. [55]).

### 3.3.2 Exemplary Discharge Notes Contain Signals for Readmission Prediction

A word cloud of discharge notes in NYU Readmission in shown in FIG. 7(a). On the left, on-readmitted patients seem to have milder diseases such as “pancreatitis” and have “friends” who can pick them up upon discharge. On the right, readmitted patients have more serious disease such as “lymphoma”, which requires frequent hospital visits for chemotherapy and radiotherapy.

3.3.3 Exemplary 30-Day all-Cause Readmission and the Hierarchy of Readmission Prediction

For example, it is possible to define readmission as 30-day all-cause readmission. That is, it is possible to say a patient is readmitted if there is a subsequent admission within 30 days.

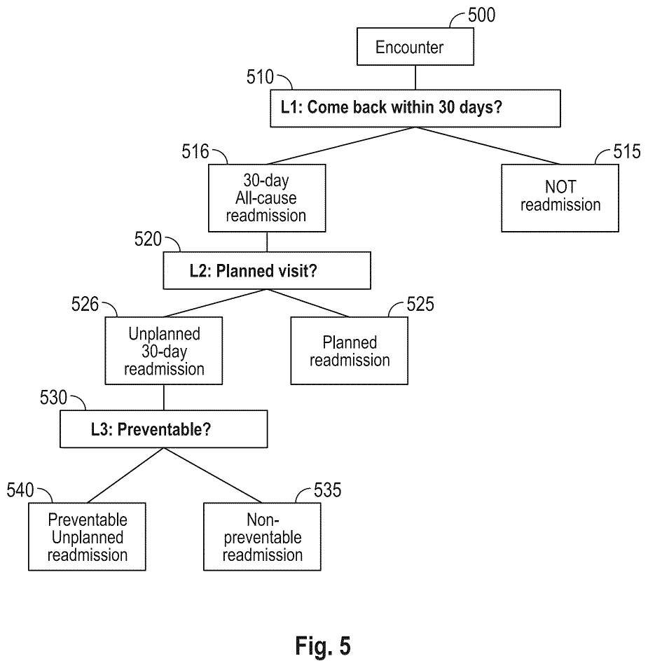

The definition of “readmission” does not solely consist of preventable read-mission (the ones that people care most about, or the yellow box after L3, as shown in FIG. 5). For example, if a patient had a successful brain surgery, went home and fell over the stairs, it is possible to stated that the patient has an “all-cause 30-day readmission” although it is not preventable.

FIG. 5 is an exemplary decision three which has three levels for predicting readmission according to exemplary embodiments. L1 label (510) was chosen because obtaining L2 (520) and L3 (530) label is expensive. At deployment, the physicians can use their judgement for L2 and L3 to filter out the false positives for “preventable, unplanned readmission”.

In a preferrable scenario, one should finetune with the “unplanned, preventable 30-day readmission” label. However, this label does not exist in the database, so it is possible to use a looser label (from L1/step 510, as shown in FIG. 5) and leave the rest of the decisions (L2/step 520 and L3/step 530) for the physicians. The L1 label covers the set of all “unplanned, preventable 30-day readmission”: that is, if a case has a positive L3 label, it must have a positive L1 label.

To get more precise labels, it is possible to recruit a team of experienced physicians to manually annotate each one of the 506, 740 cases, with potential disagreement over which cases are preventable. This annotation is expensive with ambiguity over the “preventable” label, and we think the costs outweigh the benefits. To elaborate, the current exemplary readmission prediction model (fine-tuned with L1 label) will alert unplanned, preventable 30-day readmission with some false positives (orange boxes: nonpreventable cases and planned cases). At deployment, the physician can use their judgement to filter out the false positive. For example, if the physician got alerted for a case with 3-day follow-up, we assume the physician will ignore the alert because they know the predicted readmission is planned. If one trains a model with L3 label, the benefit is that there will be fewer false positive, and the costs is expensive annotation and potentially missing preventable cases from the ambiguity of annotating “preventable” cases.

### 3.3.4 Exemplary Practical Significance of Performance Improvement

Given a large patient cohort, every 0.01% improvements could positively affect the health of real patients. For example, suppose the recall of read-mission can be improved from 78% to 80% for a cohort of 27,376 patients from January to April of 2022 (the size of NYU Readmission—Deployment, shown in Table 7) with readmission rate of 10%. That means an extra 55 high-risk patients would be identified prior to discharge. Suppose 27% of the patients' readmission are preventable (from FIG. 4b), then we could stop around 15 patients from coming back to NYU Langone with interventions (e.g., scheduling follow-up calls, delaying discharge, at-home visits). Even small improvement could prevent real patients from suffering from readmission, on which they are six times more likely to die and stay three days longer, with an additional cost of $15,000 per patient.

3.4 Exemplary Comparison of Implementation Complexity: NYUTron Vs. FHIR+RNN

To illustrate NYUTron's benefit of low-cost implementation and low-resistance deployment, here it is possible to provide and/or illustrate a comparison of developing and deploying (1) FHIR+RNN model used in [50], as outlined in their supplementary materials, nv.s. (2) NYUTron.

The following exemplary 7 steps can be used and/or required for preparing data to the FHIR format:
1. Joining data: one need to include at least the following 19 tables with 1207 total columns, as shown in Table 1. One need to write multiple sql scripts to join them together.
2. Data cleaning: it is possible to manually examine and remove fields of data with mostly null values, and fields that contain care-irrelevant information (e.g., billing). Some examples include ‘isdeleted’ and ‘lastupdatedinstant’.
3. Value mapping: it is possible to map text fields for diagnosis into standardized ICD-9 or ICD-10 codes.
4. Processing flowsheets: it is possible to sort vital signs and nursing documentation by the entry time.
5. Convert to FHIR format: for each patient, create a json file that captures their entire medical history as a sequence of events, represented by various features.
6. Further processing based on feature types. For example, if the feature is numeric value, one need to either concatenate the value with their units, or convert this value to its quantile representation. For the delta time between events, one need to choose between rounding, capping, log scale, and discretization with buckets.
7. Selecting the embedding size for each feature: either choose it as the number of unique values for that features, or do a hyperparameter search. (For high-dimensional features, doing a hyperparameter search for each feature is very expensive).

TABLE 1
Minimal Tables in NYU Datalake Required for the FHIR + RNN Approach.

| Name | # columns | Name | # columns |
|---|---|---|---|
| encounterFact | 123 | procedureDim | 50 |
| patientDim | 133 | procedureEvent | 56 |
| medicationDim | 25 | procedureOrder | 65 |
| medicationOrder | 135 | procedureTerminology | 15 |
| medicationEvent | 59 | surgicalProcedureEventFact | 48 |
| labTest | 164 | dentalProcedureEventFact | 52 |
| labComponentResult | 57 | providerDim | 70 |
| diagnosisDim | 33 | clinicalNoteFact | 39 |
| diagnosisEventFact | 48 | clinicalNoteTextFact | 13 |
| diagnosisTerminologyDim | 22 | | |

As a comparison, our language model based approach has a low-resistance data preparation with minimal manual processing and requires just 2 step:
1. Joining data: collect clinical notes from encounterFact, clinicalNoteFact, and clinicalNoteTextFact. The queried data has 2 columns: encounterkey and text. For self-supervised pretraining, the data preparation is finished. For supervised finetuning, it is possible to additionally add a column of labels.
2. Preprocessing text: train a tokenizer from the pretraining text and tokenize the finetuning text.

Apart from the difficulty of the data preparation, the exemplary approaches based on high dimensional structured data have the additional problem of being challenging to deploy. Integration with FHIR data requires the full interoperability of a potential EHR system with FHIR. While the Office of the National Coordinator for Health Information Technology has mandated FHIR interoperability by end-of-year 2022, challenges remain in real world support and compatibility. With LLM based approaches, integration can still be achieved using FHIR, but can be as simple as copying and pasting as the only required input is free text.

3.5 Exemplary Comparison of the Multifaceted Complexity of NYUTron with Traditional Clinical Predictive Model

NYUTron can be more computationally-complex and storage-complex than traditional clinical predictive model because it performs more computations and has more stored parameters.

NYUTron can be less data-complex than traditional clinical predictive model because it requires less data fusing, imputation, and feature engineering. The present disclosure demonstrated this in the exemplary rapid prototyping and implementation of four additional tasks under 1 week.

NYUTron can be less deployment-complex than traditional clinical predictive model, because they enable real-time inference as physicians write notes and require fewer labelled examples. With clinical LLMs, physicians can get real-time predictions as soon as they sign their notes in the EHR.

### 3.6 Exemplary Clinical Language Model Facilitates a Generalization Across Different Health Systems Through Local Finetuning

The following examples of across-health-system generalization through local finetuning can be provided.

The first example is Gatortron-og (from University of Florida Health) generalizes to NYU Readmission (from NYU Langone Health). In FIG. 3(b), the x-hatched line comes from finetuning Gatortron-og, a language model pre-trained with a mix of clinical text (notes from University of Florida Health) and non-clinical text (web text, wikipedia, pubmed abstracts). The finetuning data is NYU Readmission, which contains discharge notes from NYU Langone Health. FIG. 3(b) shows that with 100 and 1000 examples, Gatortron has a lower AUC than NYUTron. However, Gatortron catches up to NYUTron after 10,000 local finetuning examples.

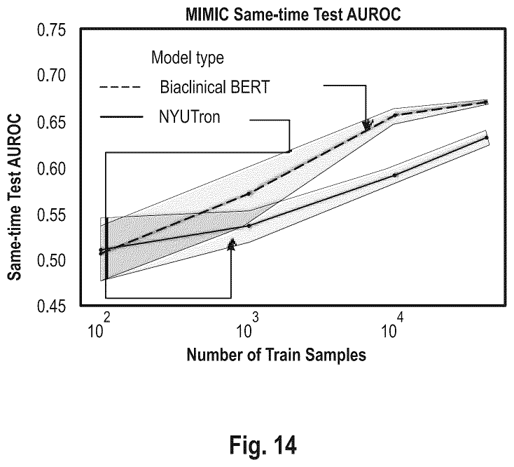

The second example is NYUTron (from NYU Langone Health) generalizes to MIMIC-III Readmission (from Beth Israel Deaconess Medical Center in Boston). It is possible to finetune and tested NYUTron on the MIMIC-III read-mission dataset, which consists of de-identified discharge notes from the Beth-Israel's ICU with binary labels for 30-day all-cause readmission. It is possible to compare NYUTron with BioClinicalBERT[56], whose pretraining data covers the MIMIC notes. FIG. 14 shows an exemplary graph indicating that at 1000 samples, NYUTron has a 3.58% higher median AUC than BioClinicalBERT (57.22% vs. 53.64%). At 10,000 samples, NYUTron has a 6.42% higher median AUC than BioClinicalBERT (65.56% vs. 59.14%). Using the full dataset (42,180 samples), NYUTron has a 3.8% higher median AUC than BioClinicalBERT (67.04% vs. 63.24%).

In particular, the exemplary graph of FIG. 14 provides an exemplary comparison of NYUTron's and BioClinicalBERT's performance on MIMIC-III Readmission according to an exemplary embodiment of the present disclosure. To test how much finetuning NYUTron needs to generalize to another health system, it is possible to finetune NYUTron and BioClinicalBERT (which has the same number of parameters and architecture as NYUTron, but pre-trained on MIMIC notes, bookcorpus, pubmed and wikipedia articles) using different subsamples of MIMIC-III readmission dataset. The dataset contains 52,726 deidentified ICU discharge notes from Boston Beth Israel Hospital with 8:1:1 train-val-test split. At 100 samples, the AUC is similar. At 1000 samples, NYUTron has a 3.58% higher median AUC than BioClinicalBERT (57.22% vs. 53.64%). At 10,000 samples, NYUTron has a 6.42% higher median AUC than BioClinicalBERT (65.56% v.s. 59.14%). Using the full dataset (42,180 samples), NYUTron has a 3.8% higher median AUC than BioClinicalBERT (67.04% vs. 63.24%). Given that NYUTron was pretrained on identified all-department notes from NYU Langone and finetuned on deidentified ICU-specific notes from Beth-Israel, this result shows that NYUTron is able to generalize to a very different health environment through local finetuning.

3.7 Exemplary Text Data May not be Less Robust than Structured Data

FIG. 3(c) shows that NYUTron is “non-robust” to changes in deployment site in the sense that: when the model is pretrained on one site, but finetuned and tested on the other site, there is a performance drop compared to doing everything locally.

However, it is possible that text-based model is not necessarily less robust than structured-data-based model. To show this, it is possible to execute the same “Manhattan-versus-Brooklyn” experiments using site-specific variants of NYU Readmission—LACE. The result is shown in Table 2. For brevity, thus, it is possible to focus on the results of Manhattan test and discuss 3 findings.

TABLE 2
Manhattan vs. Brooklyn readmission prediction
experiment using lace + xgb

Tested on

| Trained on | Brooklyn | Manhattan |
|---|---|---|
| All | 57.18% ± 0.319% | 62.70% ± 0.345% |
| Brooklyn | 58.37% ± 1.19% | 63.11% ± 1.61% |
| Manhattan | 58.11% ± 0.0213% | 64.62% ± 0.0824% |

First, Table 3 shows that when the structured data based model is trained in Brooklyn, and tested in Manhattan, there is also a performance drop (1.51% AUC, or 2.34% relative percentage drop) compared to doing everything locally.

TABLE 3
lace + xgb also has a performance drop when we vary the train site

| | Tested on |
|---|---|
| Trained on | Manhattan |
| Brooklyn | 63.11% ± 0.161% |
| Manhattan | 64.62% ± 0.0824% |

Second, the performance drop from structured data model is not necessarily smaller than the performance drop from text data model. For example, Table 4 shows that when NYUTron is pretrained in Brooklyn, but finetuned and tested in Manhattan, there is a performance drop of 0.63% AUC, or 0.73% relative percentage drop. Both NYUTron's absolute change (0.63% vs. 1.51%) and relative change (0.73% vs. 2.34%) is smaller than the observed drop from lace+xgb. Another example is shown in Table 5: Manhattan-pretrained NYUTron is finetuned in Brooklyn and tested in Manhattan. Compared to performing everything locally, there is a performance drop of 1.6% AUC, or 1.89% relative percentage drop. While NYUTron's absolute change (1.89% vs. 1.51%) is larger than lace+xgb, its relative percentage drop (1.89% vs. 2.34%) is smaller than lace+xgb.

TABLE 4
NYUTron's performance drop when we vary the pretraining site

| | Tested on |
|---|---|
| Pretrained&finetuned on | Manhattan |
| Brooklyn-Manhattan | 84.11% ± 0.09% |
| Manhattan-Manhattan | 84.74% ± 0.14% |

TABLE 5
NYUTron's performance drop when we vary the finetuning site

| Pretrained&finetuned on/Tested on | Manhattan |
|---|---|
| Manhattan-Manhattan | 84.74% ± 0.14% |
| Manhattan-Brooklyn | 83.14% ± 0.13% |

Third, it is possible to observe that the language models achieve a higher overall AUC (Table 4, Table 5) than lace+xgb (Table 3).

Together, the three finding suggests that NYUTron is not less robust than lace+xgb on readmission prediction, and that it has a better AUC than lace+xgb.

3.8 Exemplary deployment Platform—NYUTriton

Deploying machine learning models in a live healthcare environment can carry multiple considerations both technically, clinically, and ethically the full extent of which are beyond the scope of this article. There are various essays and editorials on these topics, and it is possible to include in the references several which are particularly lucid on the subject (see, e.g., Refs. [57]-[60]). It is possible to specifically focus here on the actual experience in deployment of a large language model, NYUTron, in a real-world environment and the unique considerations when working with these large models in terms of performance, security, reliability, interpretability.

Performance is likely a major focus of every software engineering project, and the optimizations were built into TensorRT and Onnx and nVidia Triton. TensorRT is an accelerated format for deep neural networks that builds in several optimizations to make models faster and more portable. NVIDIA Triton accepts TensorRT or Onnx formatted models, and facilitates their access via its REST API. It is possible to choose to run a modified, Dockerized version of NVIDIA Triton in order to take advantage of these optimizations for rapid model inferencing while utilizing on-premises hardware.

Security and monitoring can be major concerns in healthcare environments that handle the personal health information of thousands of millions of vulnerable patients. While the present system is naturally suitable to a cloud deployment, and could be done using secured communications to minimize the possibility of data breach, for security purposes one opted to utilize our own internal hardware for model serving. NYUTriton was generated to run using docker-compose or as a Helm chart for immediate and scalable deployment via Kubernetes. To facilitate monitoring, NYUTriton was integrated with Prometheus and Grafana to provide continuous monitoring by our engineering team.

Interpretable outputs is one final, additional, consideration when working with LLMs in deployment. While a consideration for medical machine learning algorithms in general, where it has been widely discussed (see, e.g., Refs. [9] and [61]), this may bear a particular significance in the case of LLMs for two reasons: (1) LLMs can be a potential universal interface for EHR analytics, and with universal inputs comes the added potential of unexpected behaviors, (2) LLMs may be complex and black-box in nature. While it is possible to perform sensitivity analysis and to look at attention weighting on inputs to attempt to understand what drives model predictions, in a real-world medical case interpretability may be frequently overrated while evidenced based evaluation is underrated. If LLMs are properly validated in prospective, randomized controlled trials (as are many medical devices), than understanding the inner workings of them is much less relevant. In line with this thinking, a randomized controlled trial of NYUTron was began, which was tied to an intervention, in order to directly assess its performance at delivering a positive impact on patient care.

### 3.9 Exemplary Potential Explanations of the Subgroup Discrepancies

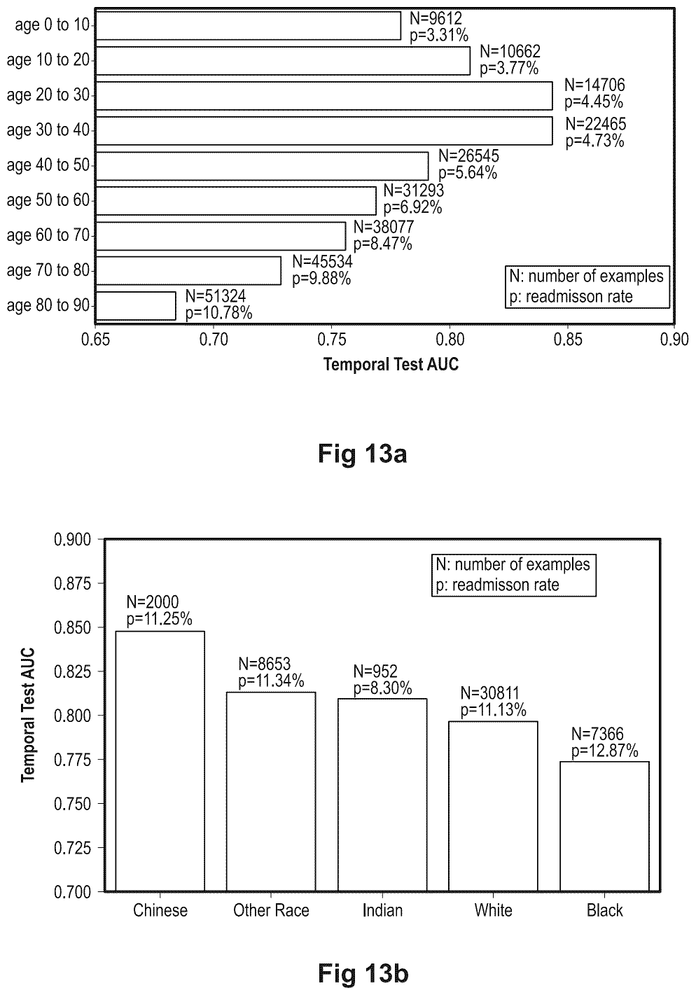

The complex data generating process of clinical notes (which depends on a variety factors such as social and medical history of patients and providers, interactions between patients and providers, and the norms of our society) makes identifying the causes of subgroup discrepancies shown in FIGS. 12(a) and 12(b) and FIGS. 13(a) and 13(b) somewhat difficult.

For example, FIGS. 12(a) and 12(b) illustrate graphs/charts providing an exemplary bias analysis stratifying NYUTron's performance by clinical departments and months according to exemplary embodiments.

In particular, FIG. 12(a) shows an exemplary chart providing a stratified analysis of NYUTron's temporal test performance by clinical department and oncological subspecialty, according to an exemplary embodiment. NYUTron performs best in the Neurology Department (AUC 90.12%), and performs worst in the Internal Medicine Department (AUC 67.95% for non-oncology specialty and AUC 63.77% for oncology specialty), with a difference of about 20% AUC. This significant variance across clinical department suggests that a more fine-grained analysis may lead to performance benefits as the model is explicitly conditioned on author department. We annotate the number of examples (N) and the readmission rate (p) for each department.

FIG. 12(b) shows a chart illustrating that the exemplary NYUTron's performance can display minor fluctuations over months according to exemplary embodiments. The average monthly test AUC of NYUTron is plotted from January 2013 to December 2021 to look for underlying monthly trends or cycles and to test the hypothesis that performance would be worst in July when new physicians start their training with a different writing style than physicians already in practice (dashed red line indicating the monthly AUC of July). The height of the bar indicates average monthly performance across the 9 years and the vertical bar indicates the standard deviation. The number of examples (N) and the readmission rate (p) are annotated for each month. July has the second lowest monthly AUC and the highest variance. Clinical notes written by new physicians is associated with the temporal shift across the months and the drop in performance in July. Average AUCs from the quarters January to March, April to June, and July to September are increasing, which may coincide with residents' rotation schedule across different clinical departments.

FIGS. 13(a) and 13(b) illustrate exemplary charts/graphs providing an exemplary bias analysis stratifying NYUTron's performance by age groups and major racial groups according to exemplary embodiments. As part of an analysis of model performance by two possible sources of bias, age and race, it is possible to perform stratified analyses of NYUTron's performance. It is possible to annotate the number of examples (N) and the readmission rate (p) for each evaluation.

In particular, the chart/graph of FIG. 13(a) shows that the temporal test based on nine bins of ages (0 to 90 years with bins of 10 year intervals) is satisfied. NYUTron performs best for patients who are 10 to 40 years old, and has declining performance by decile over the age of 40 years with the worst performance in the 80-90 years of age group. This isn't an effect of sample size, the single largest sample is age 80-90, but likely reflects complexity and comorbidity burdens being disproportionately higher with advanced age.

The chart/graph of FIG. 13(b) illustrates potential dependencies and bias by race. The five most frequent races in the dataset (White, Other Race, Black, Chinese, Indian) are identified, then the evaluation results by race are stratified. NYUTron performs best on Chinese patients and worst on Black patients with a mild variation in AUC across both groups.

Thus, the following observations can be provided:
1. Toxicity and bias in clinical texts. For example, Ref. [62] provides that different ethnic groups have different levels of recorded pain. It is possible that the provider's writings were affected by their bias towards different ethnic groups.
2. Inherent difference between subgroup distribution. For example, Ref. [63] provides hat even using self-reported numerical level of menstrual pain, Australian women have a higher level of pain than Chinese women. It is possible that these two groups naturally have different pain threshold. Another example is that hospitals with higher readmission rates have patients with “more chronic conditions, less education, fewer assets” (see, e.g., Ref. [64]), suggesting that the patient demographics may affect the distribution of readmission.
3. Complex social factors such as systematic racism. For example, it is possible that NYUTron performs worse on predicting black patients' readmission because they have a more complex medical history due to systematic racism, rendering them the more “difficult” cases for prediction.

### 3.10 Exemplary Details on Comorbidity Imputation

Charlson comorbidity index (CCI) quantifies the severity of a patient's health condition based on the patient's history of chronic disease and severe condition. The index chooses a set of chronic diseases (e.g., congestic heart failure, liver disease) and assigns a positive score for each chronic disease. The final index sums over all the score, and a larger index indicates a more severe health condition. The index can help physicians predict patient outcomes.

The conventional calculation of CCI requires data collection and manual entry. Using EHR, we can automate the process by first identifying the history of chronic disease using ICD (International Classification of Diseases) diagnosis codes, and then assigning scores based on the ICD codes.

However, the ICD codes are missing for certain patients (in our case, 22% of the encounter). For example, patients who transferred from an external health system with a separate EHR will have no past ICD codes. In this case, we want to impute the comorbidity index. This setting is different than common imputation tasks, in that not partial, but all structured data are missing. Motivated by the richness of care-relevant information in clinical notes, we propose to impute CCI using clinical notes and language models.

### 3.11 Exemplary Extended Data

TABLE 6
Sizes and pretrain corpora for LLMs. We test 6 types of LLMs with
different model sizes and different pretraining corpora. Exemplary
corporate are listed as well as model parameter counts to facilitate
ease of comparison. Further, one key distinction between web-
wiki + bio + clinical and NYUTron, clinical can be that the former
was stripped of identifying information while the latter was not.

| | Model | Clinical | Biomedical | General |
|---|---|---|---|---|
| Model | Size | Text | Text | Text |
| NYUTron, clinical (ours) | 109 m | 4.1B real | 0 | 0 |
| web-wiki + bio + clinical | 345 m | 82B real | 6B | 2.5B |
| web-wiki + bio | 109 m | 0 | 18B | 3.3B |
| web-wiki | 109 m | 0 | 0 | 3.3B |
| random-init | 109 m | 0 | 0 | 0 |

TABLE 7
Detailed statistics of datasets. An exemplary comprehensive pretraining
dataset (NYU Notes) was generated with two site-specific variants
(NYU Notes - Manhattan/Brooklyn) as discussed in the Methods
section. For readmission prediction, an exemplary finetuning
dataset (NYU Readmission) was also generated with two site-specific
variants (NYU Readmission Manhattan/Brooklyn), one structured-
data variant (NYU Read-mission - LACE), and a deployment test
set (NYU Readmission - Deployment) that was sampled in real-
time as part of our prospective trial. To test the breadth of
NYUTron's applicability, 4 exemplary tasks were added (NYU
Mortality, NYU Binned LOS, NYU Comorbidity, NYU Insurance denial)
with their respective structured-data variant ( NYU Mortality -
SAPS2 + APACHE2, NYU Binned LOS - Lisbon Portugal, NYU
Insurance Denial - Claim forms). NYU Comorbidity has no structured-
data variant because the task is to impute comorbidity index
with the lack of structured icd codes. Finally, an exemplary
Named Entity Recognition (NER) dataset was provided for testing
how well NYUTron generalizes to different clinical predictive
tasks using non-NYU data.

| Dataset | # Notes | # Patients | # Words |
|---|---|---|---|
| NYU Notes | 7,247,694 | 387,144 | 4,112,249,482 |
| NYU Notes - Manhattan | 4,342,602 | 256,217 | 2,381,466,993 |
| NYU Notes - Brooklyn | 1,337,352 | 104,521 | 1,102,078,012 |
| NYU Readmission | 506,740 | 413,845 | 487,395,462 |
| NYU Readmission - | 296,519 | 240,824 | 253,622,053 |
| Manhattan | | | |
| NYU Readmission - | 113,275 | 94,653 | 142,767,957 |
| Brooklyn | | | |
| NYU Readmission - | 0 | 413,845 | 0 (structured |
| LACE | | | data) |
| NYU Readmission - | 29,287 | 27,376 | 34,669,963 |
| Deployment | | | |
| NYU Mortality | 469,162 | 371,922 | 484,467,141 |
| NYU Mortality - | 0 | 371,922 | 0 (structured |
| SAPS2 + APACHE2 | | | data) |
| NYU Binned LOS | 469,162 | 371,922 | 484,467,141 |
| NYU Binned LOS - | 0 | 371,922 | 0 (structured |
| Lisbon Portugal | | | data) |
| NYU Comorbidity | 403,579 | 327,039 | 422,485,417 |
| NYU Insurance Denial | 55,791 | 54,563 | 51,270,256 |
| NYU Insurance Denial - | 0 | 54,563 | 0 (structured |
| Claim forms | | | data) |
| i2b2-2012-NER | 310 | ≤310 | 636K |

The Ninth Revision of the International Classification of Diseases (ICD-9) is a standardized coding system used to classify health conditions. It is used for billing, tracking individual patient conditions, and for epidemiology. The highly detailed and technical nature of the codes and their associated medical conditions make it difficult for humans to accurately record them. Researchers have explored the use of neural networks, particularly language models, for automated ICD-9 code assignment. However, the imbalanced distribution of ICD-9 codes can lead to poor performance. One solution can be to use domain knowledge to incorporate a useful prior. Exemplary embodiments of the present disclosure show that while the correlation bias can worsen overall performance, the effect on individual class can be negative or positive.¹ Performance on classes that are more imbalanced and less correlated with other codes can be more sensitive to incorporating the correlation bias. This may suggest that while the correlation bias has potential to improve ICD-9 code assignment in certain cases, the applicability criteria need to be more carefully considered.

Electronic Health Records (EHRs) contain patient information in the form of clinical notes, structured data tables, and biomedical imaging and time series. For easy tracking and analysis of health data across different healthcare systems, and critically for billing purposes, hospitals and insurance companies assign codes of a standardized coding system to characterize the clinical conditions of patients. Wrong code assignments may result in billing issues that increase patients' expenses substantially, misdiagnosis, and poor tracking of population level health conditions nationally. The Ninth Revision of the International Classification of Diseases (ICD-9) is a system used worldwide to classify and code diseases, injuries, and other health conditions. There were extensive efforts studying the automated assignment of ICD-9 codes to health records and relevant documents (see, e.g., Yan et al., 2022).

With recent developments in NLP, there has been a focus on the use of neural networks (see, e.g., Yu et al., 2019; Mullenbach et al., 2018; and Teng et al., 2020). One recent direction is in the use of language models. Originally introduced in BERT (see, e.g., Devlin et al., 2019), the recipe of pretraining and finetuning of language models has shown promising performance in many tasks. Researchers have applied BERT for assigning ICD-9 codes from medical documents (see, e.g., Huang et al., 2022; Pascual et al., 2021; and Zhang et al., 2020). However, BERT and other encoder-based language models perform poorly on ICD-9 code assignment (see, e.g., Yan et al., 2022).

One challenge is the extremely imbalanced distribution of ICD-9 codes. Following the distribution of medical conditions in the real world, some codes occur frequently while other codes may appear only once (see, e.g., Yan et al., 2022). It is difficult for models to correctly predict minority codes because few samples exist in the dataset (Sun et al., 2009). A proposed solution is to incorporate domain knowledge that provides useful priors for the minority codes (see, e.g., Bai and Vucetic, 2019; Wang et al., 2020; and Zeng et al., 2019).

With the systems, methods, apparatus and computer-accessible medium according to the exemplary embodiments of the present disclosure, it can be understood that one useful prior for ICD-9 code assignment is the correlation between ICD9 codes and other relevant coding systems. For example, it is possible to term other relevant coding systems auxiliary tasks because language models in exemplary embodiments predict codes from these systems in addition to ICD9 codes. The auxiliary tasks are Current Procedural Terminology (CPT) codes and Diagnosis-Related Group (DRG) codes. This correlation prior stems from the domain knowledge that labels from other coding systems give information about ICD-9 codes. For example, patients who underwent artery bypass surgeries (CPT code 33533) are likely to have heart failures (ICD-9 code 428.0). To test this likely indication, the systems, methods, apparatus and computer-accessible medium according to the exemplary embodiments of the present disclosure can investigate the effect of multitasking on correlated auxiliary tasks and encouraging similar label correlations between training labels and model predictions through regularization. The systems, methods, apparatus and computer-accessible medium according to the exemplary embodiments of the present disclosure can be used to show that 1) on average, utilizing correlations hurts language models' performance on predicting ICD-9 codes from discharge summaries, 2) for each ICD-9 code, utilizing correlations may hurt or help, 3) ICD-9 codes that are more imbalanced and less correlated with auxiliary tasks can experience larger performance changes (both positive and negative) from incorporating the correlation prior. Exemplary findings suggest that the correlation prior has the potential to improve predictions of certain ICD-9 codes, but this method can suffer from instability when the main task has an imbalanced label distribution and a weak correlation with auxiliary tasks.

Exemplary Domain knowledge: According to exemplary embodiments of the present disclosure, one exemplary useful prior for ICD-9 codes is its hierarchical structure. For example, a high-level code (e.g., 428.0 heart failure) encompasses its corresponding low-level codes (e.g., 428.1 left heart failure, 428.2 systolic heart failure). Tsai et al. (2019) incorporated this hierarchical prior and improved models' performance on predicting imbalanced ICD-9 codes.

Exemplary CorrLoss: CorrLoss is a regularization technique (Rieger et al., 2022) that encourages consistent label correlations between ground truth and predictions. Rieger et al. (2022) uses CorrLoss on the facial affect recognition task to integrate the correlation priors for facial movements. Corrloss can be used in any domain where correlation between prediction targets provides a useful signal. Thus, it is possible to adopt Corrloss to integrate information of the correlations between different kinds of diagnosis and procedure codes.

### Exemplary Methods

Exemplary Task overview: the systems, methods, apparatus and computer-accessible medium according to the exemplary embodiments of the present disclosure can formulate the task of code assignment into a multilabel text classification task because each patient has multiple codes corresponding to their discharge summaries. For example, each binary label in the task can correspond to a specific code. Formally, the classifier of exemplary embodiments aims to approximate the probability p(y₁, . . . , yₙ|x), where each yᵢ is an ICD-9 code and x is a discharge summary.

Exemplary Correlation Prior the systems, methods, apparatus and computer-accessible medium according to the exemplary embodiments of the present disclosure can provide that correlations between ICD-9 and other coding systems can be a useful prior for ICD-9 code assignment and choose to incorporate the prior in two ways.

First, in exemplary embodiments, the auxiliary tasks of predicting other medical codes (e.g., CPT) can be added. Formally, exemplary embodiments can train a classifier to approximate

$$ρ ( y , z \vert x ) = ρ ( y \vert x ) ρ ( z \vert x , y ) , \qquad ( 1 )$$

where y is a sequence of ICD-9 codes (the main task), z is a sequence of other medical codes (the auxiliary task), and x is a discharge summary. The domain knowledge, according to exemplary embodiments, can assume that the absolute correlation abs(ρ(y, z)|x)>0, so y, z are not conditionally independent given x and ρ(z|x, y)/=ρ(z|x). This is desirable because otherwise, the difficulty of the task is strictly increasing from learning ρ(y|x) to learning ρ(y|x) p(z|x).

In exemplary embodiments of the present disclosure, there can be benefits associated with Equation 1, and the trade-off can be unclear a priori. One exemplary benefit is that extra dependency information from ρ(z|x, y) could potentially simplify learning ρ(y, z|x). One drawback can be that the additional prediction targets z could worsen the curse of dimensionality. Whether the benefit outweighs the drawback can be difficult to determine without running a controlled experiment.

Second, the systems, methods, apparatus and computer-accessible medium according to the exemplary embodiments of the present disclosure can use CorrLoss to encourage similar label correlation patterns between training and predictions. Formally, exemplary embodiments can add a regularization term c=^{L}_{/i=j}c(dᵢ, dⱼ). Each summation term scales with a correlation difference:

$$c ( {d}_{i} , {d}_{j} ) ∝ \vert {ρ ( {d}_{i} , {d}_{j} )}_{{y}_{train}} - {ρ ( {d}_{i} , {d}_{j} )}_{\hat{y}} \vert , \qquad ( 2 )$$

where dᵢ, dⱼ are different classes, ρ(dᵢ, dⱼ)ᵥ is the correlation between class dᵢ and dⱼ in a vector v, yₜᵣₐᵢₙ is the training labels, y\hat{ } is the predicted labels, and ρ is the Pearson correlation function.

TABLE 8
Macro F1 scores of experiments, in which procedure
ICD-9 is the main task, on MIMIC-III-50 test set.

| | | | PROC + | PROC + | PROC + |
|---|---|---|---|---|---|
| | | PROC | CPT | DRG | DIAG |
| ClinicalBERT | original | 0.4528 | 0.397 | 0.3939 | 0.408 |
| CorrLoss | | 0.4037 | 0.3594 | 0.3272 | 0.363 |
| RoBERTa | original | 0.4421 | 0.4009 | 0.3884 | 0.4116 |
| CorrLoss | | 0.3736 | 0.3236 | 0.2816 | 0.3692 |
| Longformer | original | 0.4712 | 0.4227 | 0.3886 | 0.4219 |
| CorrLoss | | 0.4139 | 0.335 | 0.212 | 0.3549 |

For each model, the best F1 score is in bold. PROC means procedure ICD-9. DIAG means diagnosis ICD-9. PROC + CPT means that procedure ICD-9 is the main task and CPT is the auxiliary task.

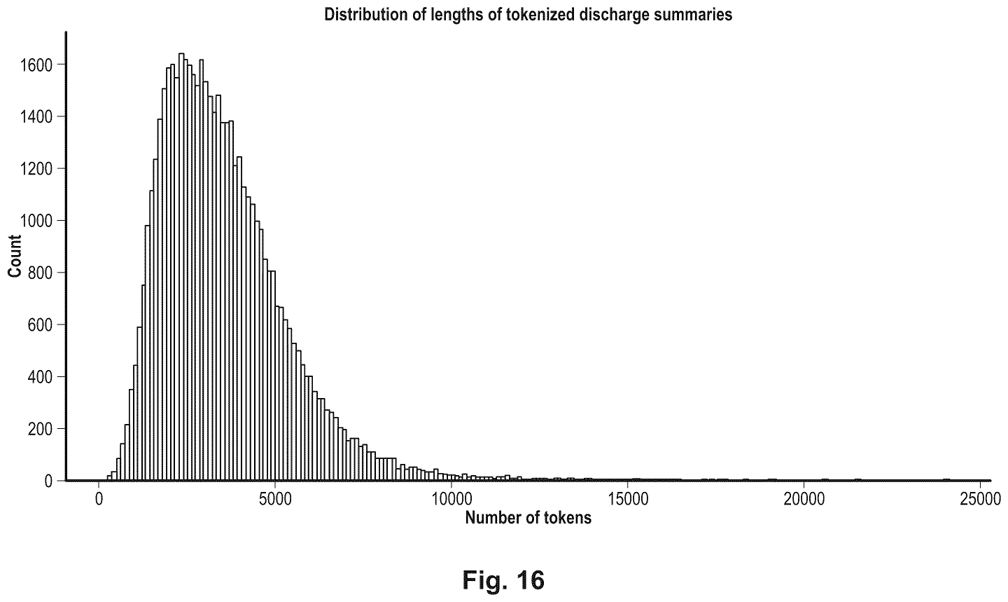

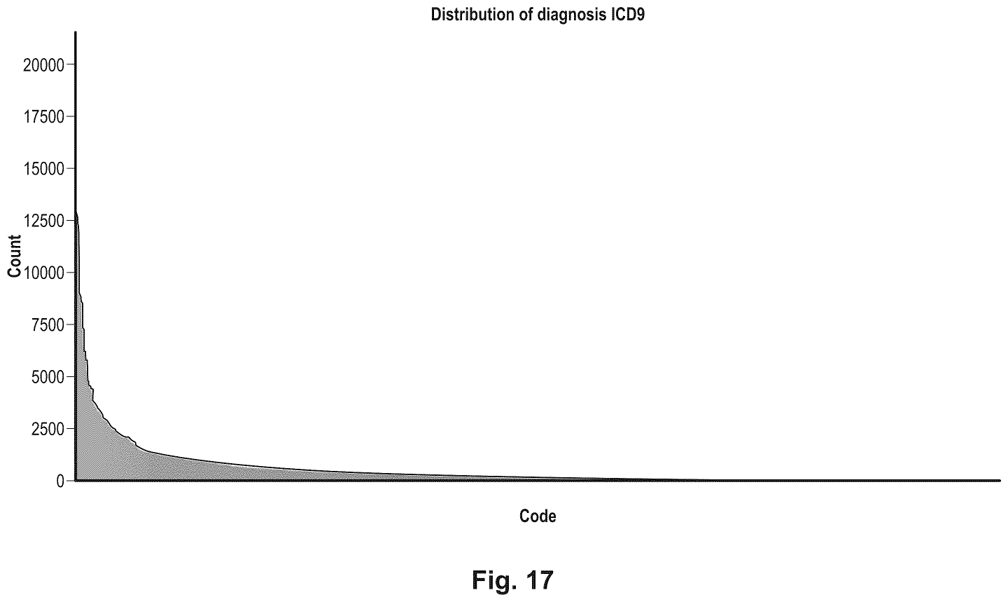

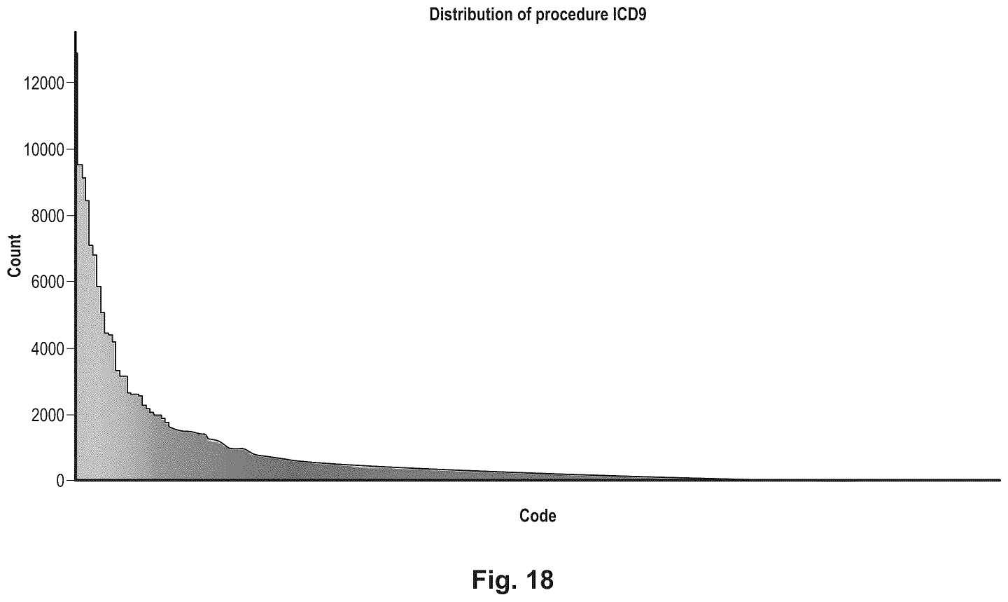

Exemplary Dataset: The systems, methods, apparatus and computer-accessible medium according to the exemplary embodiments of the present disclosure built two datasets from the Medical Information Mart for Intensive Care III (MIMICIII) (Johnson et al., 2016), a database of EHRs. The first dataset, subsequently referred to as “MIMICIII”, contains examples of each patient's discharge summary, and associated diagnosis and procedure codes (diagnosis ICD-9, procedure ICD-9, CPT, and DRG). Because this dataset is extremely imbalanced, exemplary embodiments can further select the top 50 most frequently used codes for each kind of coding system to construct a second dataset that can represent a more ideal scenario. Following the convention of related literature, exemplary embodiments may call this dataset “MIMIC-III-50” (Vu et al., 2020; Luo et al., 2021; Li and Yu, 2020). FIG. 16 shows exemplary statistics illustrating the distribution of lengths of tokenized discharge summaries in MIMIC-III dataset. FIG. 17 shows exemplary statistics illustrating the distribution of diagnosis ICD-9. There are 6918 diagnosis ICD-9 codes and 6062 Codes occur less than or equal to 100 times in the MIMIC-III dataset. For the sake of clarity, these codes are not included in the exemplary statistics. FIG. 18 shows exemplary statistics illustrating the distribution of procedure ICD-9 codes. There are 2011 procedure ICD-9 codes and 1767 Codes occur less than or equal to 100 times in MIMIC-III dataset. For the sake of clarity, these codes are not included in the exemplary statistics.

Exemplary Models and Evaluation: The systems, methods, apparatus and computer-accessible medium according to the exemplary embodiments of the present disclosure can use ClinicalBERT (Alsentzer et al., 2019), RoBERTa (Liu et al., 2019), Longformer (Beltagy et al., 2020). The variant of ClinicalBERT used in exemplary embodiments can be Bio+Discharge Summary BERT model because it was further trained on discharge summaries from MIMIC-III after initialized from BioBERT (Lee et al., 2020).

The systems, methods, apparatus and computer-accessible medium according to the exemplary embodiments of the present disclosure can use RoBERTa because it is a variant of vanilla BERT that was trained differently to improve its performance on a range of NLP tasks.

The systems, methods, apparatus and computer-accessible medium according to the exemplary embodiments of the present disclosure can use Longformer because it can handle long text sequences. BERT and many BERT-based models cannot handle text sequences longer than 512 tokens. Many tokenized discharge summaries are text sequences longer than 512 tokens and Longformer can benefit from more complete understandings of discharge summaries.

Each model represents a different improvement on top of vanilla BERT: ClinicalBERT improves through domain-specific pretraining; RoBERTa improves through tuning training setup; and Longformer improves through incorporating more information from the input. With these models, exemplary embodiments cover a significant part of the improvement spectrum, which shows that the pattern presented by exemplary embodiments is generalizable to different models.

The systems, methods, apparatus and computer-accessible medium according to the exemplary embodiments of the present disclosure can use the macro F1 as a metric for comparison because this metric treats all classes equally, which means minority codes are as important as majority codes in evaluation (see, e.g., Branco et al., 2016; Sun et al., 2009; and Ferri et al., 2009). Because it is an imbalanced classification, the default threshold of 0.5 may not be suitable (see, e.g., Zhou and Liu, 2006; and Zou et al., 2016). Instead, the systems, methods, apparatus and computer-accessible medium according to the exemplary embodiments of the present disclosure can tune the threshold according to the precision-recall curve to maximize the F1 score for each individual label.

### Exemplary Experiments

To test whether the correlation prior is useful for ICD code assignment, exemplary embodiments can incorporate multitasking (Equation 1) and CorrLoss (Equation 2) into the model and check if they improve performance. Specifically, the systems, methods, apparatus and computer-accessible medium according to the exemplary embodiments of the present disclosure can review two main tasks (diagnosis ICD-9 codes and procedure ICD-9 codes). For each main task, the systems, methods, apparatus and computer-accessible medium according to the exemplary embodiments of the present disclosure can add one of the three auxiliary tasks: DRG codes, CPT codes, and the other ICD-9 codes (for diagnosis ICD-9 code, the auxiliary task can be procedure ICD-9 code, and vice versa). The systems, methods, apparatus and computer-accessible medium according to the exemplary embodiments of the present disclosure can train both main-task-only models and multitasking models with and without CorrLoss.

### Exemplary Results

Exemplary Multitasking and CorrLoss can hurt performance on MIMIC-III-50 and may not significantly impact performance on MIMIC-III. Table 8 shows exemplary macro-F1 score on procedure ICD-9 of the MIMIC-III-50 dataset according to exemplary embodiments of the present disclosure. The systems, methods, apparatus and computer-accessible medium according to the exemplary embodiments of the present disclosure can observe two patterns for each language model. First, in exemplary embodiments, adding auxiliary tasks always decreases the performance of models in comparison to predicting main tasks only. Second, in exemplary embodiments, regularizing with CorrLoss always decreases the performance of models in comparison to not using CorrLoss. The same pattern exists for predicting diagnosis ICD-9 of the MIMIC-III-50 dataset. However, on the full MIMIC-III dataset, multitasking and CorrLoss do not impact models' performance significantly, as illustrated in exemplary tables 9-11.

TABLE 9
Macro F1 scores of experiments, in which procedure
ICD-9 is the main task, on full MIMIC-III test set.

| | | | PROC + | PROC + | PROC + |
|---|---|---|---|---|---|
| | | PROC | CPT | DRG | DIAG |
| ClinicalBERT | original | 0.0098 | 0.0094 | 0.0091 | 0.0097 |
| | CorrLoss | 0.0102 | 0.0099 | 0.0088 | 0.0087 |
| RoBERTa | original | 0.0097 | 0.0089 | 0.0087 | 0.0088 |
| | CorrLoss | 0.0095 | 0.0095 | 0.0098 | 0.0089 |
| Longformer | original | 0.0088 | 0.0088 | 0.0095 | 0.0085 |
| | CorrLoss | 0.0094 | 0.0085 | 0.0091 | 0.0078 |

TABLE 10
Macro F1 scores of experiments, in which diagnosis
ICD-9 is the main task, on full MIMIC-III test set.

| | | | DIAG + | DIAG + | DIAG + |
|---|---|---|---|---|---|
| | | DIAG | CPT | DRG | PROC |
| ClinicalBERT | original | 0.0068 | 0.0066 | 0.0066 | 0.0067 |
| | CorrLoss | 0.0066 | 0.0069 | 0.0069 | 0.0068 |
| RoBERTa | original | 0.0069 | 0.0065 | 0.0062 | 0.0065 |
| | CorrLoss | 0.0071 | 0.0071 | 0.0066 | 0.0065 |
| Longformer | original | 0.0072 | 0.0069 | 0.007 | 0.0071 |
| | CorrLoss | 0.007 | 0.0068 | 0.0076 | 0.0071 |

TABLE 11
Macro F1 scores of experiments, in which diagnosis
ICD-9 is the main task, on MIMIC-III-50 test set.

| | | | DIAG + | DIAG + | DIAG + |
|---|---|---|---|---|---|
| | | DIAG | CPT | DRG | PROC |
| ClinicalBERT | original | 0.3755 | 0.3296 | 0.3351 | 0.3351 |
| | CorrLoss | 0.3235 | 0.2966 | 0.2947 | 0.2992 |
| RoBERTa | original | 0.3851 | 0.3255 | 0.3307 | 0.3341 |
| | CorrLoss | 0.3143 | 0.2822 | 0.2713 | 0.2939 |
| Longformer | original | 0.4408 | 0.349 | 0.3544 | 0.3552 |
| | CorrLoss | 0.3364 | 0.2963 | 0.2906 | 0.3027 |

### Exemplary Analysis

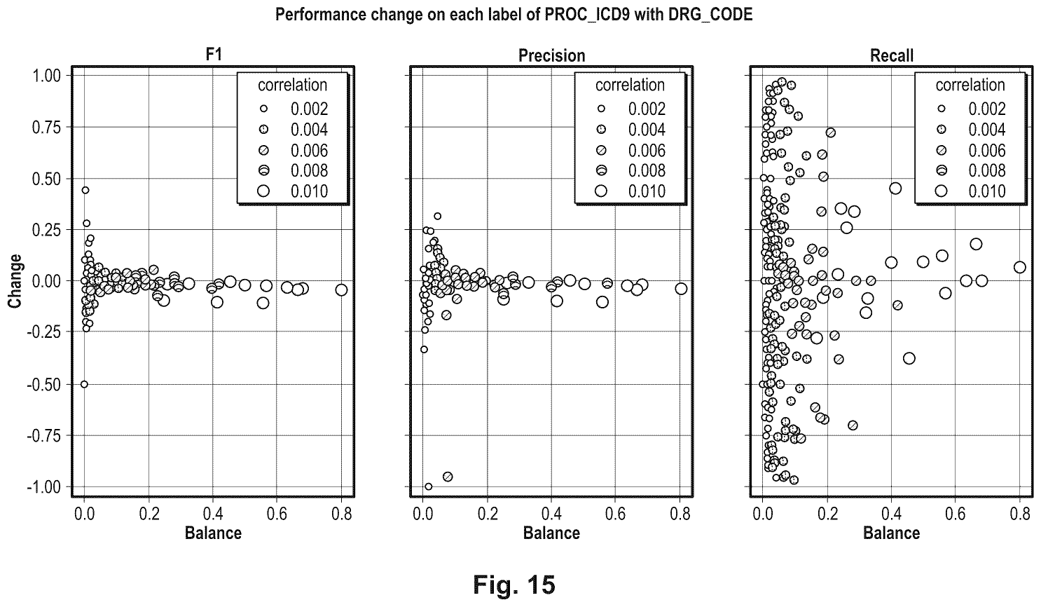

Since the macro F1 score does not show significant changes from multitasking and CorrLoss on the full MIMIC-III dataset, exemplary embodiments of the present disclosure can investigate whether the performance changes for individual labels. Specifically, exemplary embodiments can analyze how label imbalance (measured by Shannon entropy) and label correlation (measured by the average absolute Pearson correlation coefficient between each main task label and all auxiliary task labels) affect the model's performance. For individual ICD-9 code, according to exemplary embodiments of the present disclosure, incorporating the correlation prior may hurt or help. FIG. 15 shows exemplary graphs indicating that there exist labels with both negative and positive performance changes.

### Exemplary Shannon Entropy

$$H ( X ) = - {i = 1}_{X}^{} p ( {x}_{i} ) {log}_{2} p ( {x}_{i} ) \qquad ( 3 )$$

In this equation, H(X) represents the entropy of a label X with possible outcomes x₁, x₂, . . . , xₙ. In the context of the exemplary embodiments of the present disclosure, n=2 because a label only has two possible outcomes: 1 (positive) or 0 (negative). The term p(xᵢ) represents the probability of the i-th outcome, and the logarithm is taken with base 2 to give the result in units of bits. The sum is taken over all possible outcomes of X. With only two possible outcomes, a label's Shannon entropy will be close to 1 if it is balanced, and will be close to 0 if it is imbalanced.

### Exemplary Representation of Correlations

$$C ( a , B ) = \frac{{L}_{b ϵ B} \vert P ( a , b ) \vert}{card ( B )} \qquad ( 4 )$$

In this equation, C(a, B) represents the correlations between a label of the main task a and a set containing labels of the auxiliary task. For each label of the auxiliary task b∈B, |P(a, b)| represents the absolute value of the Pearson correlation coefficient between a and b. card(B) is the cardinality of B (i.e. the number of labels in B).

Exemplary labels that are more imbalanced and less correlated to auxiliary labels can experience larger changes. The graphs shown in FIG. 15, according to exemplary embodiments of the present disclosure, can indicate two relationships: (1) more balanced labels (closer to the right) can have less performance changes (spread of dots on the y axis), (2) labels that are more correlated with the auxiliary task (darker dots) can have less performance changes (spread along the y axis). Tables 14-18 illustrate exemplary plots of different tasks and setups according to exemplary embodiments of the present disclosure, and reveal similar patterns.

TABLE 12
The percentages of positive macro F1 score changes on the
top 50 most balanced procedure ICD-9 labels and on the bottom
50 least balanced procedure ICD-9 labels, with different
auxiliary tasks and models. CorrLoss is not included.

| | | top50 | bottom50 |
|---|---|---|---|
| ClinicalBERT | +CPT | 0.333 | 0.273 |
| | +DRG | 0.28 | 0.413 |
| | +DIAG | 0.3 | 0.387 |
| RoBERTa | +CPT | 0.4 | 0.3 |
| | +DRG | 0.393 | 0.353 |
| | +DIAG | 0.313 | 0.287 |
| Longformer | +CPT | 0.34 | 0.427 |
| | +DRG | 0.34 | 0.28 |
| | +DIAG | 0.347 | 0.307 |

TABLE 13
The percentages of positive macro F1 score changes on the
top 50 most balanced diagnosis ICD-9 labels and on the
bottom 50 least balanced diagnosis ICD-9 labels, with
different auxiliary tasks and models. CorrLoss is not
included in all experiments we examine in this table.

| | | top50 | bottom50 |
|---|---|---|---|
| ClinicalBERT | +CPT | 0.453 | 0.32 |
| | +DRG | 0.54 | 0.293 |
| | +PROC | 0.48 | 0.38 |
| RoBERTa | +CPT | 0.48 | 0.313 |
| | +DRG | 0.507 | 0.307 |
| | +PROC | 0.48 | 0.333 |
| Longformer | +CPT | 0.5 | 0.32 |
| | +DRG | 0.48 | 0.393 |
| | +PROC | 0.433 | 0.287 |

TABLE 14
The percentages of positive macro F1 score changes on
the top 50 most balanced procedure ICD-9 labels and on
the bottom 50 least balanced procedure ICD-9 labels,
with different auxiliary tasks and models. CorrLoss is
included in all experiments we examine in this table.

| | | top50 | bottom50 |
|---|---|---|---|
| ClinicalBERT | +CPT | 0.347 | 0.36 |
| | +DRG | 0.327 | 0.313 |
| | +DIAG | 0.273 | 0.28 |
| RoBERTa | +CPT | 0.32 | 0.32 |
| | +DRG | 0.353 | 0.36 |
| | +DIAG | 0.273 | 0.22 |
| Longformer | +CPT | 0.353 | 0.367 |
| | +DRG | 0.28 | 0.293 |
| | +DIAG | 0.307 | 0.26 |

TABLE 15
The percentages of positive macro F1 score changes on
the top 50 most balanced diagnosis ICD-9 labels and on
the bottom 50 least balanced diagnosis ICD-9 labels,
with different auxiliary tasks and models. CorrLoss is
included in all experiments we examine in this table.

| | | top50 | bottom50 |
|---|---|---|---|
| ClinicalBERT | +CPT | 0.413 | 0.307 |
| | +DRG | 0.533 | 0.28 |
| | +PROC | 0.487 | 0.293 |
| RoBERTa | +CPT | 0.46 | 0.3 |
| | +DRG | 0.493 | 0.373 |
| | +PROC | 0.473 | 0.34 |
| Longformer | +CPT | 0.453 | 0.293 |
| | +DRG | 0.487 | 0.34 |
| | +PROC | 0.5 | 0.307 |

TABLE 16
The percentages of positive macro F1 score changes on the
top 50 procedure ICD-9 labels that are most correlated with
the auxiliary task and on the bottom 50 procedure ICD-9
labels that are least correlated with the auxiliary task,
with different auxiliary tasks and models. CorrLoss is not
included in all experiments we examine in this table.

| | | top50 | bottom50 |
|---|---|---|---|
| ClinicalBERT | +CPT | 0.467 | 0.32 |
| | +DRG | 0.307 | 0.373 |
| | +DIAG | 0.367 | 0.287 |
| RoBERTa | +CPT | 0.387 | 0.267 |
| | +DRG | 0.413 | 0.407 |
| | +DIAG | 0.32 | 0.307 |
| Longformer | +CPT | 0.427 | 0.367 |
| | +DRG | 0.34 | 0.307 |
| | +DIAG | 0.42 | 0.307 |

TABLE 17
The percentages of positive macro F1 score changes on the
top 50 diagnosis ICD-9 labels that are most correlated with
the auxiliary task and on the bottom 50 diagnosis ICD-9
labels that are least correlated with the auxiliary task,
with different auxiliary tasks and models. CorrLoss is not
included in all experiments we examine in this table.

| | | top50 | bottom50 |
|---|---|---|---|
| ClinicalBERT | +CPT | 0.507 | 0.333 |
| | +DRG | 0.493 | 0.287 |
| | +PROC | 0.473 | 0.347 |
| RoBERTa | +CPT | 0.48 | 0.247 |
| | +DRG | 0.513 | 0.36 |
| | +PROC | 0.46 | 0.347 |
| Longformer | +CPT | 0.487 | 0.313 |
| | +DRG | 0.493 | 0.34 |
| | +PROC | 0.427 | 0.313 |

TABLE 18
The percentages of positive macro F1 score changes on the
top 50 diagnosis ICD-9 labels that are most correlated with
the auxiliary task and on the bottom 50 diagnosis ICD-9
labels that are least correlated with the auxiliary task,
with different auxiliary tasks and models. CorrLoss is included
in all experiments we examine in this table.

| | | top50 | bottom50 |
|---|---|---|---|
| ClinicalBERT | +CPT | 0.467 | 0.373 |
| | +DRG | 0.52 | 0.3 |
| | +PROC | 0.46 | 0.333 |
| RoBERTa | +CPT | 0.493 | 0.32 |
| | +DRG | 0.52 | 0.433 |
| | +PROC | 0.473 | 0.253 |
| Longformer | +CPT | 0.46 | 0.32 |
| | +DRG | 0.513 | 0.467 |
| | +PROC | 0.453 | 0.34 |

In both extreme scenarios (imbalanced label, small correlation with auxiliary labels) and ideal scenarios (balanced labels, high correlation with auxiliary labels), exemplary embodiments reveal that incorporating correlation is more likely to hurt than help. Table 12 shows that for the top 50 most balanced labels and the bottom 50 least balanced labels, if exemplary embodiments utilize correlations (with multitasking and CorrLoss), the percentage of positive F1 score changes is always less than 50%. Table 19 shows that for the top 50 labels that are most correlated with the auxiliary tasks and the bottom 50 labels that are least correlated with the auxiliary tasks, in exemplary embodiments of the present disclosure, utilizing correlations also leads to <50% positive F1 score change.

TABLE 19
The percentages of positive macro F1 score changes on the top
50 procedure ICD-9 labels that are most correlated with the
auxiliary task and on the bottom 50 procedure ICD-9 labels
that are least correlated with the auxiliary task, with different
auxiliary tasks and models. CorrLoss is included.

| | | top50 | bottom50 |
|---|---|---|---|
| ClinicalBERT | +CPT | 0.333 | 0.327 |
| | +DRG | 0.32 | 0.327 |
| | +DIAG | 0.293 | 0.247 |
| RoBERTa | +CPT | 0.487 | 0.333 |
| | +DRG | 0.373 | 0.387 |
| | +DIAG | 0.267 | 0.293 |
| Longformer | +CPT | 0.433 | 0.327 |
| | +DRG | 0.28 | 0.273 |
| | +DIAG | 0.333 | 0.24 |

### Exemplary Discussion

Since, according to exemplary embodiments of the present disclosure, multitasking and CorrLoss worsen language models' overall performance, it contradicts a hypothesis of exemplary embodiments that the correlations between ICD-9 codes and other medical codes would be a useful prior. Nevertheless, the performance changes on individual labels can be more nuanced and show potential for improving prediction of certain ICD-9 codes.

Recent advances in large language models have led to renewed interest in natural language processing in healthcare using the free text of clinical notes. One distinguishing characteristic of clinical notes is their long time span over multiple long documents. The exemplary unique structure of clinical notes creates a new design choice: when the context length for a language model predictor is limited, which part of clinical notes should be chosen as the input? Existing studies either choose the inputs with domain knowledge or simply truncate them. Exemplary embodiments of the present disclosure propose a framework to analyze the sections with high predictive power. Using MIMIC-III, exemplary embodiments show that: 1) predictive power distribution can be different between nursing notes and discharge notes and 2) combining different types of notes could improve performance when the context length is large. Exemplary embodiments suggest that a carefully selected sampling function can facilitate more efficient information extraction from clinical notes.

Electronic Health Records (EHR) enable the development of language model based clinical predictor, which takes in clinical notes to predict patient outcomes. Clinical notes in EHR exhibit two unique characteristics. 1) Clinical notes cover a long time span (from a few weeks to over a year), which results in their sparsity of information-rich sections. 2) Clinical notes also tend to be long: many discharge notes could take up to 10,000 tokens, which makes using the entire note as model input computationally expensive. 3) The strong noise level in the medical notes (usually due to the domain specific abbreviations and typos) also poses a challenge to extract information effectively.

These exemplary distinguishing characteristics of clinical notes lead to a new design choice: when the context length is limited due to the constrained compute or model architecture, what parts of clinical notes should be sampled to maximize the model's performance? The systems, methods, apparatus and computer-accessible medium according to the exemplary embodiments of the present disclosure can provide a framework to subsample text sections with high predictive power.

Empirically, the systems, methods, apparatus and computer-accessible medium according to the exemplary embodiments of the present disclosure can explore the distribution of predictive power over clinical note types and sections by searching over these variables. The systems, methods, apparatus and computer-accessible medium according to the exemplary embodiments of the present disclosure can indicate that 1) the predictive power distribution can be different between nursing notes and discharge notes: the predictive power can be stronger at the beginning and end of discharge notes, while uniform within nursing notes. 2) The effect of combining sections from different types of notes can improve the performance when the context size is large, but can harm the performance when the context size is small.

### Exemplary Related Work

Existing methods for subsampling clinical notes for the BERT-based model are mostly based on domain knowledge. For instance, Yang et al. (2022) and Darabi et al. (2020) choose discharge notes as they summarize patients' visits. Thapa et al. (2022) chooses the notes within three days before a cutoff time in consideration of timeliness. While these assumptions are based on domain knowledge, they require human input and may not generalize. Thus, exemplary embodiments are interested in exploring a data-driven sampling choice without assumptions of expert inputs. Another related, but orthogonal approach to the limited context length problem is note aggregation. Instead of subsampling notes, Huang et al. (2019) propose to feed everything to the model, one maximum context length at a time, and aggregate the outputs for the final prediction. In their work, notes of one patient are split into a partition of subsequences, and the patient's re-admission risk is obtained by taking a weighted average of probabilities computed from each subsequence. This method's compute cost scales with the aggregated sequence length, which can be expensive for records with long clinical notes. In contrast, methods according to exemplary embodiments aim to find one single information-rich segment as input.

### Further Exemplary Method, System, Apparatus and Computer-Accessible Medium

The systems, methods, apparatus and computer-accessible medium according to the exemplary embodiments of the present disclosure can formalize the prediction task as follows: given a set of clinical notes x associated with an admission record, exemplary embodiments want to predict the class label y which is the patient outcome of interest. Ideally, exemplary embodiments can train a classifier f_{w*} to approximate p(y|x). The optimal parameter is

$${w}^{*} = arg {max}_{w} m ( {f}_{w} ( x ) , y ) ,$$

where m is a metric function of interest. Nevertheless, due to the computational constraint, the systems, methods, apparatus and computer-accessible medium according to the exemplary embodiments of the present disclosure can reduce the input size via a sampling function s_{θ} so that s_{θ}(x) fits the input length limit and preserves information. Empirically, the optimal parameters are

$${w}^{*} , {θ}^{*} = \begin{matrix}arg max \\ w , θ\end{matrix} m ( {f}_{w} ( {s}_{θ} ( x ) , y ) )$$

According to various exemplary embodiments, a sample function s_{θ} has a higher predictive power if m(f_{w}(s_{θ}(x), y)) is larger.

While current works chose s_{θ} based on prior medical knowledge or simply fix it as a truncation function, the systems, methods, apparatus and computer-accessible medium according to the exemplary embodiments of the present disclosure can providing and/or utilize different sampling functions s_{θ} to make the most out of the limited context length with the highest predictive power. For example, s and θ can be searched manually, instead of using learning algorithms.

### Exemplary Experimental Setup

The systems, methods, apparatus and computer-accessible medium according to the exemplary embodiments of the present disclosure can indicate that for 30-day all-cause readmission prediction, there exists an alternative sampling function that facilitates similar or better performance than the commonly used “truncated discharge notes”. For example, the systems, methods, apparatus and computer-accessible medium according to the exemplary embodiments of the present disclosure can focus on a parameterized sampling function with 2 variables: 1) which section of tokens to include, 2) what type(s) of clinical notes to use.

Exemplary Model: The systems, methods, apparatus and computer-accessible medium according to the exemplary embodiments of the present disclosure can finetune two clinical language models. The first is Clinical-BERT (Alsentzer et al., 2019), which continued to pretrain BERT using approximately 2 million notes from MIMIC-III and has a maximum sequence length of 512. The second is the ClinicalLongformer (Li et al., 2022), which continued to pretrain Longformer (Beltagy et al., 2020) with MIMIC-III notes and enables input of up to 4096 tokens. In exemplary embodiments, both models can be finetuned to predict the probability of 30-day all-cause readmission: that is, whether the patient will be re-admitted to the hospital within 30 days of their discharge dates.

Exemplary Dataset: The systems, methods, apparatus and computer-accessible medium according to the exemplary embodiments of the present disclosure can use the discharge notes and nursing notes in the noteevent table of the MIMIC-III database (Johnson et al., 2016). In exemplary embodiments, there can be 40,000 de-identified admission records available to use after filtering out all admission records without nursing notes and discharge notes. The admission records can be split into 75% train, 12.5% validation, and 12.5% test sets. Other types of medical notes such as physician notes can be excluded from consideration in exemplary embodiments due to their scarcity in the database.

Exemplary Preprocessing: The systems, methods, apparatus and computer-accessible medium according to the exemplary embodiments of the present disclosure can preprocess the dataset with the following approach: First, admission records with missing discharge notes or missing nursing notes can be eliminated. Then, for each remaining admission record, the nursing notes associated with that record can be sorted according to their timestamp. According to exemplary embodiments, the first and last created nursing notes for each admission can be selected and concatenated with the discharge notes of the same admission record to produce the clinical note set for every admission. Lastly, the systems, methods, apparatus and computer-accessible medium according to the exemplary embodiments of the present disclosure can clean the datasets by removing the de-identification patterns in the clinical notes, which usually occupy a lot of tokens.

Exemplary Sliding Window: To extract different sections of the clinical notes, exemplary embodiments of the present disclosure can use a sliding window technique. Let n be the window's width. Let l be the total number of tokens of the text. The window can be placed based on an input parameter p∈[0, 1] indicating the location of the midpoint of the window, where the window interval is

$$[ lp - n / 2 , lp + n / 2 ] .$$

In the case where lp−n/2<0, the systems, methods, apparatus and computer-accessible medium according to the exemplary embodiments of the present disclosure can shift the window backward so that the front of the window aligns with the beginning of the input tokens. In the case where lp+n/2>l exemplary embodiments can shift the window forward to let the back of the window match the end of the tokens. In addition, when l<n, the systems, methods, apparatus and computer-accessible medium according to the exemplary embodiments of the present disclosure can ignore the input p and pad the tokens to maximum input length n.

The systems, methods, apparatus and computer-accessible medium according to the exemplary embodiments of the present disclosure can try 11 different values of p (0.0, 0.1, . . . 1.0) for ClinicalBERT and 2 values of p (0.0 and 1.0) for ClincialLongformer along with an additional fragmented window trial p=both which looks into the first n/2 and last n/2 tokens of the input text. Similarly, when l<n, exemplary embodiments can simply pad the sequence to the window's length.

Exemplary Mixing Notes: To control different types of clinical notes, exemplary embodiments of the present disclosure can use the following options: 1) first nursing note, 2) last nursing note, 3) discharge note, 4) first nursing notes+discharge note, 5) last nursing notes+discharge notes. For options with two types of notes, n/2 tokens can be allocated by exemplary embodiments to each type, and three values for p₁ and p₂ each (0.0, 1.0 and both) can be used to select n/2 tokens from each type of note, resulting in 9 possible input parameter combinations.

### Exemplary Results

### Exemplary Different Sections in Nursing Notes and Discharge Notes

The systems, methods, apparatus and computer-accessible medium according to the exemplary embodiments of the present disclosure can finetune ClinicalBERT and ClinicalLongformer on different sections of nursing and discharge notes. Exemplary embodiments may use sliding windows to extract a sequence of tokens that meets the model's maximum sequence length. For example, three key observations can be revealed.

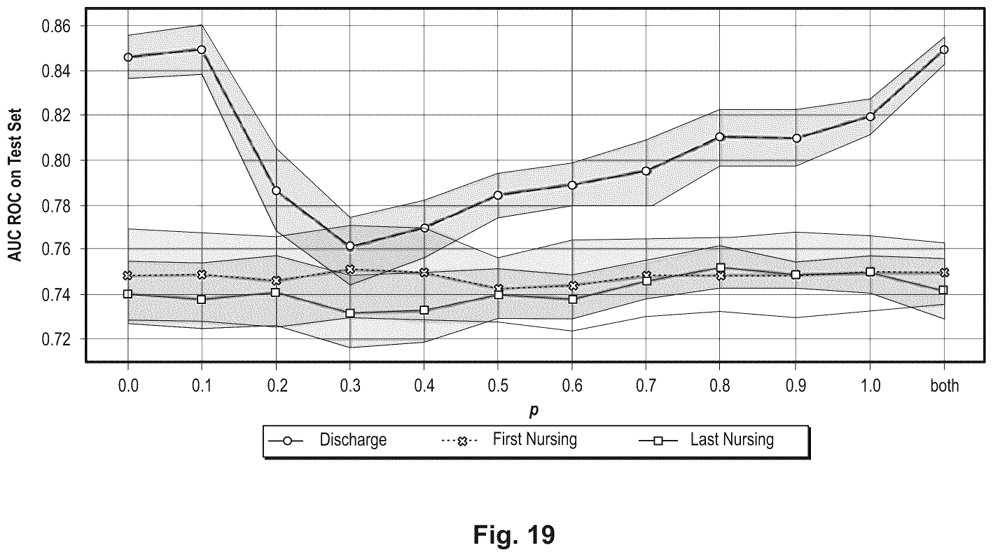

Exemplary Different Types of Clinical Notes Can Show Disparate Predictive Power Distributions Over Text Sections. As shown in exemplary FIG. 19, the discharge notes, according to exemplary embodiments, show quite uneven predictive power distribution, where the beginning (p=0.0) and end (p=1.0) sections of the text provide strong predictive power while the middle sector (0.2≤p≤0.5) shows a significant dip in predictive power. In contrast, the predictive power of the nursing notes, according to exemplary embodiments, turns out to be uniformly distributed: using different sections of the nursing notes (0.0≤p≤1.0) does not make a significant difference. The systems, methods, apparatus and computer-accessible medium according to the exemplary embodiments of the present disclosure can reveal that this discrepancy may stem from the domain knowledge that discharge notes are more structured than nursing notes: they often start with basic descriptions of the patient information and ends with suggestions for the patients, whereas nursing notes often have multiple types of information mixed together throughout the text.

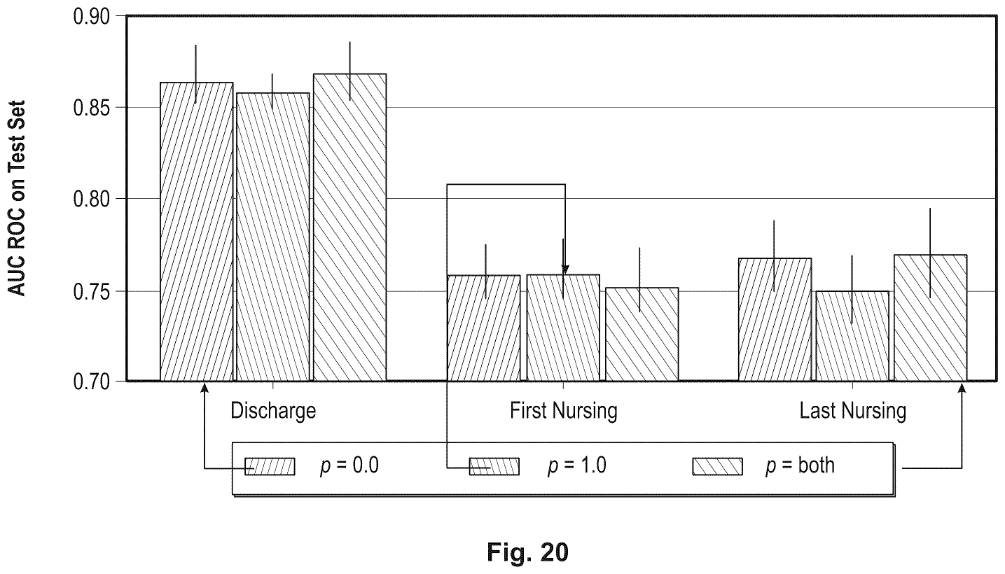

Exemplary Nursing Notes May Provide Modest Predictive Power. In exemplary embodiments of the present disclosure, nursing notes can produce decent re-admission prediction results: according to FIG. 19 and FIG. 20, although their predictive power is not as strong as discharge notes (which are typically written right before patients leave the hospital), they consistently achieve AUC ROC scores of over 0.7 which indicates modest predictability (Schneeweiss et al., 2001). Moreover, according to exemplary embodiments, the first nursing notes (FIGS. 19 and 20) of each admission provide similar predictive power as compared to the last nursing notes (FIGS. 19 and 20), indicating the possibility of re-admission risk evaluation at the early stage of the admission. This finding of the systems, methods, apparatus and computer-accessible medium according to the exemplary embodiments of the present disclosure can be especially valuable from the perspective of intervention, as it is more practical to decide whether the patient should be discharged at the time before the discharge note is written. Also, the abundance of nursing notes makes them a suitable alternative for re-admission risk evaluation tasks when discharge notes are unavailable.

Exemplary Preservation of the Beginning Tokens Is Not the Only Option. It is generally assumed that when the available input tokens are limited, the leading tokens of each clinical note should be used. Nevertheless, the systems, methods, apparatus and computer-accessible medium according to the exemplary embodiments of the present disclosure can show that for discharge notes, spending half of the available tokens on the beginning section and spending the remaining half on the end section (p=both) can achieve slightly better performance (AUC ROC of 0.849 versus 0.845 for ClinicalBERT, 0.869 versus 0.864 for ClinicalLongformer) as compared to using the leading token only (p=0.0). The systems, methods, apparatus and computer-accessible medium according to the exemplary embodiments of the present disclosure can show that this helps as it avoids the weakly predictive middle sector of the clinical notes.

Exemplary Combining Sections from Different Types

the systems, methods, apparatus and computer-accessible medium according to the exemplary embodiments of the present disclosure can combine text sections from two different types of clinical notes and finetune ClinicalBERT and ClinicalLongformer. This can help the systems, methods, apparatus and computer-accessible medium according to the exemplary embodiments of the present disclosure investigate the question: when the amount of available tokens is fixed, does combining information from different clinical notes work better than using discharge notes only? Since the systems, methods, apparatus and computer-accessible medium according to the exemplary embodiments of the present disclosure show that discharge notes provide strong predictive power, the systems, methods, apparatus and computer-accessible medium according to further exemplary embodiments of the present disclosure can only investigate the note type combinations that include discharge notes (first nursing+discharge, last nursing+discharge).

### Exemplary Effect of Allocating Tokens to Different Types of Clinical Notes Depends on the Context Size.

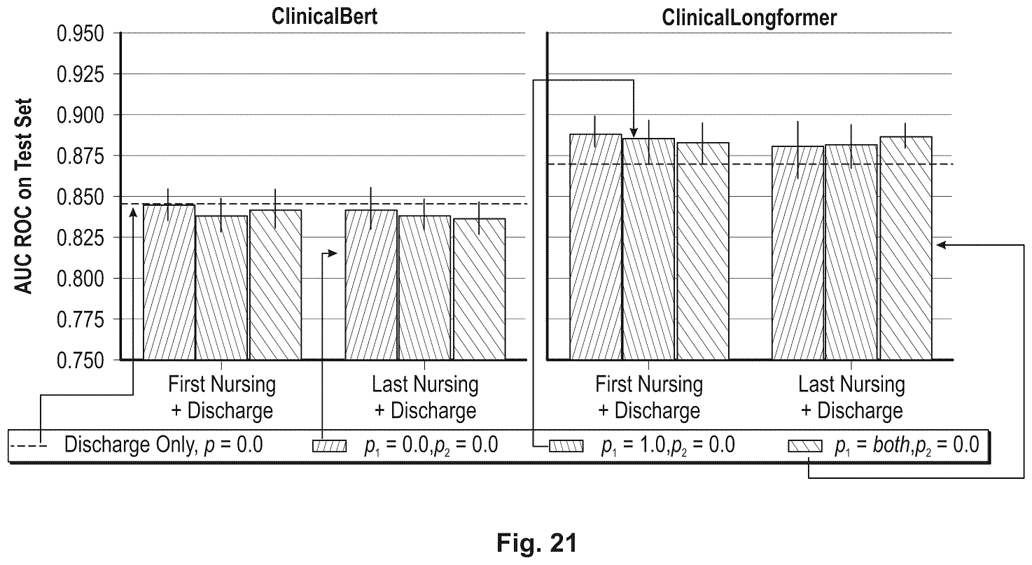

With the systems, methods, apparatus and computer-accessible medium according to the exemplary embodiments of the present disclosure, when the context size is relatively large (ClinicalLongformer, as shown in the right side of FIG. 21), allocating the available tokens to different types of clinical notes (different bars in the graph of FIG. 21) leads to improvements in performance. The baseline (dashed line) uses discharge notes only and has a lower AUC ROC (0.013 to 0.019) than models according to exemplary embodiments finetuned with combined notes. However, when the context is small (Clinical BERT, as shown in the left side of FIG. 21), distributing the already limited number of tokens to different clinical notes can hurt the performance: the AUC ROC of ClinicalBERT according to exemplary embodiments finetuned with mixed notes can fall below the baseline performance by −0.009 to −0.001. This can be related to the uneven predictive power distribution in discharge notes: if there are already a sufficient number of tokens covering the most informative sections of the discharge notes, the rest of the discharge notes might not be as informative as the prior nursing notes.

### Exemplary Discussion

Findings, the systems, methods, apparatus and computer-accessible medium according to the exemplary embodiments of the present disclosure can indicate that when the input size is constrained, a carefully selected sampling function that chooses the text with high predictive power could benefit model performance. Specifically on the task of readmission prediction from MIMIC-III notes, the systems, methods, apparatus and computer-accessible medium according to the exemplary embodiments of the present disclosure can show that the predictive power varies across note types and note sections. This insight can facilitate a more efficient information extraction from long and noisy clinical notes, which can be beneficial when the computing resource is limited and the context length needs to be controlled.

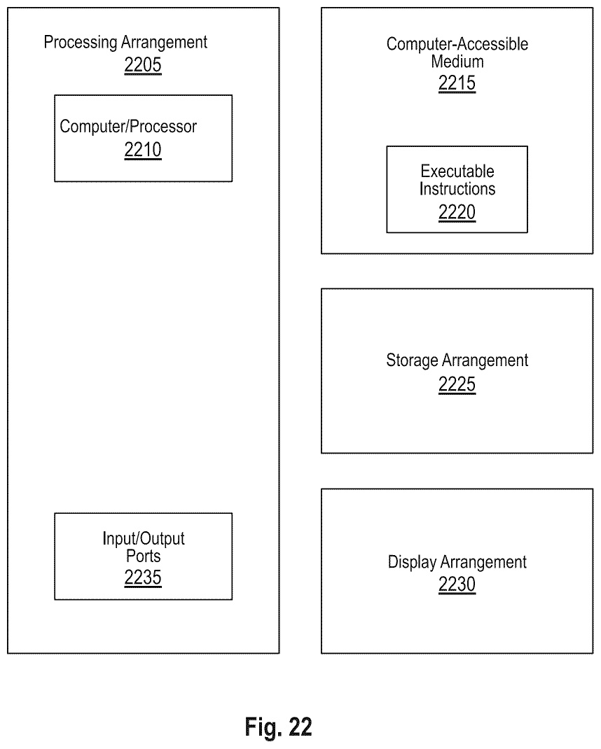

FIG. 22 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 2205. Such processing/computing arrangement 2205 can be, for example entirely or a part of, or include, but not limited to, a computer/processor 2210 that can include, for example one or more microprocessors, and use instructions stored on a computer-accessible medium (e.g., RAM, ROM, hard drive, or other storage device).

As shown in FIG. 22, for example a computer-accessible medium 2215 (e.g., as described herein above, a storage device such as a hard disk, floppy disk, memory stick, CD-ROM, RAM, ROM, etc., or a collection thereof) can be provided (e.g., in communication with the processing arrangement 2205). The computer-accessible medium 2215 can contain executable instructions 2220 thereon. In addition or alternatively, a storage arrangement 2225 can be provided separately from the computer-accessible medium 2215, which can provide the instructions to the processing arrangement 2205 so as to configure the processing arrangement to execute certain exemplary procedures, processes, and methods, as described herein above, for example.

Further, the exemplary processing arrangement 2205 can be provided with or include an input/output ports 2235, which can include, for example a wired network, a wireless network, the internet, an intranet, a data collection probe, a sensor, etc. As shown in FIG. 22, the exemplary processing arrangement 2205 can be in communication with an exemplary display arrangement 2230, which, according to certain exemplary embodiments of the present disclosure, can be a touch-screen configured for inputting information to the processing arrangement in addition to outputting information from the processing arrangement, for example. Further, the exemplary display arrangement 2230 and/or a storage arrangement 2225 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference, in their entireties:
[1] Gage, B. F., van Walraven, C., Pearce, L., Hart, R. G., Koudstaal, P. J., Boode, B. S. P., Petersen, P.: Selecting Patients With Atrial Fibrillation for Anticoagulation: Stroke Risk Stratification in Patients Taking Aspirin. Circulation 110(16), 2287-2292 (2004). https://doi.org/10.1161/01.CIR. 0000145172.55640.93
[2] Child, C. G., Turcotte, J. G.: Surgery and portal hypertension. Major Problems in Clinical Surgery 1, 1-85 (1964)
[3] Pugh, R. N. H., Murray-Lyon, I. M., Dawson, J. L., Pietroni, M. C., Williams, R.: Transection of the oesophagus for bleeding oesophageal varices. British Journal of Surgery 60(8), 646-649 (2005). https://doi.org/10.1002/bjs.1800600817
[4] Wells, P., Hirsh, J., Anderson, D., Lensing, A. A., Foster, G., Kearon, C., Weitz, J., D'Ovidio, R., Cogo, A., Prandoni, P., Girolami, A., Ginsberg, J.: Accuracy of clinical assessment of deep-vein thrombosis. The Lancet 345(8961), 1326-1330 (1995). https://doi.org/10.1016/S0140-6736(95) 92535-X
[5] Tomasev, N., Glorot, X., Rae, J. W., Zielinski, M., Askham, H., Saraiva, A., Mottram, A., Meyer, C., Ravuri, S., Protsyuk, I., Connell, A., Hughes, C. O., Karthikesalingam, A., Cornebise, J., Montgomery, H., Rees, G., Laing, C., Baker, C. R., Peterson, K., Reeves, R., Hassabis, D., King, D., Suleyman, M., Back, T., Nielson, C., Ledsam, J. R., Mohamed, S.: A clinically applicable approach to continuous prediction of future acute kid-ney injury. Nature 572(7767), 116-119 (2019). https://doi.org/10.1038/s41586-019-1390-1
[6] Wu, N., Phang, J., Park, J., Shen, Y., Huang, Z., Zorin, M., Jastrzebski, S., Fevry, T., Katsnelson, J., Kim, E., Wolfson, S., Parikh, U., Gad-dam, S., Lin, L. L. Y., Ho, K., Weinstein, J. D., Reig, B., Gao, Y., Toth, H., Pysarenko, K., Lewin, A., Lee, J., Airola, K., Mema, E., Chung, S., Hwang, E., Samreen, N., Kim, S. G., Heacock, L., Moy, L., Cho, K., Geras, K. J.: Deep Neural Networks Improve Radiologists' Performance in Breast Cancer Screening. IEEE Transactions on Medical Imaging 39(4), 1184-1194 (2020). https://doi.org/10.1109/TMI.2019.2945514
[7] Liang, H., Tsui, B. Y., Ni, H., Valentim, C. C. S., Baxter, S. L., Liu, G., Cai, W., Kermany, D. S., Sun, X., Chen, J., He, L., Zhu, J., Tian, P., Shao, H., Zheng, L., Hou, R., Hewett, S., Li, G., Liang, P., Zang, X., Zhang, Z., Pan, L., Cai, H., Ling, R., Li, S., Cui, Y., Tang, S., Ye, H., Huang, X., He, W., Liang, W., Zhang, Q., Jiang, J., Yu, W., Gao, J., Ou, W., Deng, Y., Hou, Q., Wang, B., Yao, C., Liang, Y., Zhang, S., Duan, Y., Zhang, R., Gibson, S., Zhang, C. L., Li, O., Zhang, E. D., Karin, G., Nguyen, N., Wu, X., Wen, C., Xu, J., Xu, W., Wang, B., Wang, W., Li, J., Pizzato, B., Bao, C., Xiang, D., He, W., He, S., Zhou, Y., Haw, W., Goldbaum, M., Tremoulet, A., Hsu, C. N., Carter, H., Zhu, L., Zhang, K., Xia, H.: Evaluation and accurate diagnoses of pediatric diseases using artificial intelligence. Nature Medicine 25(3), 433-438 (2019). https://doi.org/10.1038/s41591-018-0335-9
[8] AIX-COVNET, Roberts, M., Driggs, D., Thorpe, M., Gilbey, J., Yeung, M., Ursprung, S., Aviles-Rivero, A. I., Etmann, C., McCague, C., Beer, L., Weir-McCall, J. R., Teng, Z., Gkrania-Klotsas, E., Rudd, J. H. F., Sala, E., Schönlieb, C.-B.: Common pitfalls and recommendations for using machine learning to detect and prognosticate for COVID-19 using chest radiographs and CT scans. Nature Machine Intelligence 3(3), 199-217 (2021). https://doi.org/10.1038/s42256-021-00307-0
[9] Kelly, C. J., Karthikesalingam, A., Suleyman, M., Corrado, G., King, D.: Key challenges for delivering clinical impact with artificial intelligence. BMC Medicine 17(1), 195 (2019). https://doi.org/10.1186/s12916-019-1426-2
[10] Gaube, S., Suresh, H., Raue, M., Merritt, A., Berkowitz, S. J., Lermer, E., Coughlin, J. F., Guttag, J. V., Colak, E., Ghassemi, M.: Do as AI say: Susceptibility in deployment of clinical decision-aids. npj Digital Medicine 4(1), 31 (2021). https://doi.org/10.1038/s41746-021-00385-9
[11] Devlin, J., Chang, M.-W., Lee, K., Toutanova, K.: BERT: Pre-training of Deep Bidirectional Transformers for Language Understanding. Proceedings of the 2019 Conference of the North American Chapter of the Association for Computational Linguistics: Human Language Technologies 1, 4171-4186 (2019). https://doi.org/10.18653/v1/Ni9-1423
[12] Brown, T. B., Mann, B., Ryder, N., Subbiah, M., Kaplan, J., Dhariwal, P., Neelakantan, A., Shyam, P., Sastry, G., Askell, A., Agarwal, S., Herbert-Voss, A., Krueger, G., Henighan, T., Child, R., Ramesh, A., Ziegler, D. M., Wu, J., Winter, C., Hesse, C., Chen, M., Sigler, E., Litwin, M., Gray, S., Chess, B., Clark, J., Berner, C., McCandlish, S., Radford, A., Sutskever, I., Amodei, D.: Language Models are FewShot Learners. Advances in Neural Information Processing Systems 33, 1877-1901 (2020)
[13] Kaplan, J., McCandlish, S., Henighan, T., Brown, T. B., Chess, B., Child, R., Gray, S., Radford, A., Wu, J., Amodei, D.: Scaling Laws for Neural Language Models. arXiv (2020)
[14] Chen, T., Guestrin, C.: XGBoost: A Scalable Tree Boosting System. In: Proceedings of the 22nd ACM SIGKDD International Conference on Knowledge Discovery and Data Mining, pp. 785-794 (2016). https://doi.org/10.1145/2939672.2939785
[15] Le Gall, J.-R.: A New Simplified Acute Physiology Score (SAPS II) Based on a European/North American Multicenter Study. JAMA: The Journal of the American Medical Association 270(24), 2957 (1993). https://doi.org/10.1001/jama.1993.03510240069035
[16] Knaus, W. A., Draper, E. A., Wagner, D. P., Zimmerman, J. E.: APACHE II: A severity of disease classification system. Critical Care Medicine 13(10), 818-829 (1985)
[17] Charlson, M. E., Pompei, P., Ales, K. L., MacKenzie, C. R.: A new method of classifying prognostic comorbidity in longitudinal studies: Development and validation. Journal of Chronic Diseases 40(5), 373-383 (1987). https://doi.org/10.1016/0021-9681(87)90171-8
[18] A Data-driven Approach to Predict Hospital Length of Stay—A Portuguese Case Study: In: Proceedings of the 16th International Conference on Enterprise Information Systems, pp. 407-414. SCITEPRESS—Science and Technology Publications, Lisbon, Portugal (2014). https://doi.org/10.5220/0004892204070414
[19] Johnson, M., Albizri, A., Harfouche, A.: Responsible Artificial Intelligence in Healthcare: Predicting and Preventing Insurance Claim Denials for Economic and Social Wellbeing. Information Systems Frontiers (2021). https://doi.org/10.1007/sI0796-021-10137-5
[20] van Walraven, C., Wong, J., Forster, A. J.: LACE+ index: Extension of a validated index to predict early death or urgent readmission after hospital discharge using administrative data. Open Medicine 6(3), 80-90 (2012)
[21] Center for Disease Control: What Is C. Diff?U.S. Department of Health & Human Services (2022). https://www.cdc.gov/cdiff/what-is.html
[22] Yang, G., Cao, M., Jiang, L. Y., Liu, X. C., Cheung, A. T. M., Weiss, H., Kurland, D., Cho, K., Oermann, E. K.: Language Model Classifier Aligns Better with Physician Word Sensitivity than XGBoost on Readmission Prediction. arXiv (2022)
[23] Singhal, K., Azizi, S., Tu, T., Mahdavi, S. S., Wei, J., Chung, H. W., Scales, N., Tanwani, A., Cole-Lewis, H., Pfohl, S., Payne, P., Seneviratne, M., Gamble, P., Kelly, C., Scharli, N., Chowdhery, A., Mansfield, P., y Arcas, B. A., Webster, D., Corrado, G. S., Matias, Y., Chou, K., Gottweis, J., Tomasev, N., Liu, Y., Rajkomar, A., Barral, J., Semturs, C., Karthike-salingam, A., Natarajan, V.: Large Language Models Encode Clinical Knowledge. arXiv (2022)
[24] Bolton, E., Hall, D., Yasunaga, Y., Lee, T., Manning, C., Liang, P.: Pub-MedGPT 2.7BElliot Bolton and David Hall and Michihiro Yasunaga and Tony Lee and Chris Manning and Percy Liang. Technical report, Stanford University (December 2022)
[25] Hoffmann, J., Borgeaud, S., Mensch, A., Buchatskaya, E., Cai, T., Rutherford, E., Casas, D. d. L., Hendricks, L. A., Welbl, J., Clark, A., Hennigan, T., Noland, E., Millican, K., van den Driessche, G., Damoc, B., Guy, A., Osindero, S., Simonyan, K., Elsen, E., Rae, J. W., Vinyals, O., Sifre, L.: Training Compute-Optimal Large Language Models (2022). https://doi.org/10.48550/ARXIV.2203.15556
[26] Charlson, M.: Charlson Comorbidity Index (CCI). MDCalc. https://www.mdcalc.com/calc/3917/charlson-comorbidity-index-cci
[27] Sun, W., Rumshisky, A., Uzuner, O.: Annotating temporal information in clinical narratives. Journal of Biomedical Informatics 46, 5-12 (2013). https://doi.org/10.1016/j.jbi.2013.07.004
[28] Johnson, A. E. W., Pollard, T. J., Shen, L., Lehman, L.-w. H., Feng, M., Ghassemi, M., Moody, B., Szolovits, P., Anthony Celi, L., Mark, R. G.: MIMIC-III, a freely accessible critical care database. Scientific Data 3(1), 160035 (2016). https://doi.org/10.1038/sdata.2016.35
[29] van Walraven, C., Dhalla, I. A., Bell, C., Etchells, E., Stiell, I. G., Zarnke, K., Austin, P. C., Forster, A. J.: Derivation and validation of an index to predict early death or unplanned readmission after discharge from hospital to the community. Canadian Medical Association Journal 182(6), 551-557 (2010). https://doi.org/10.1503/cmaj.091117
[30] Sundararajan, V., Henderson, T., Perry, C., Muggivan, A., Quan, H., Ghali, W. A.: New ICD-10 version of the Charlson comorbidity index predicted in-hospital mortality. Journal of Clinical Epidemiology 57(12), 1288-1294 (2004). https://doi.org/10.1016/j.jclinepi.2004.03.012
[31] Honnibal, M., Montani, I.: spaCy 2: Natural Language Understanding with Bloom Embeddings, Convolutional Neural Networks and Incremen-tal Parsing. Unpublished (2017)
[32] Wolf, T., Debut, L., Sanh, V., Chaumond, J., Delangue, C., Moi, A., Cistac, P., Rault, T., Louf, R., Funtowicz, M., Davison, J., Shleifer, S., von Platen, P., Ma, C., Jernite, Y., Plu, J., Xu, C., Le Scao, T., Gugger, S., Drame, M., Lhoest, Q., Rush, A.: Transformers: State-of-the-Art Natural Language Processing. In: Proceedings of the 2020 Conference on Empirical Methods in Natural Language Processing: System Demonstrations, pp. 38-45. Association for Computational Linguistics, Online (2020). https://doi.org/10.18653/v1/2020.emnlp-demos. 6. https://aclanthology.org/2020.emnlp-demos.6
[33] Rajbhandari, S., Rasley, J., Ruwase, O., He, Y.: ZeRO: Memory Optimizations Toward Training Trillion Parameter Models. In: Proceedings of the International Conference for High Performance Computing, Networking, Storage and Analysis, pp. 1-16. IEEE Press, Atlanta, Georgia (2020)
[34] Loshchilov, I., Hutter, F.: Decoupled Weight Decay Regularization (2017). https://doi.org/10.48550/ARXIV.1711.05101
[35] Kingma, D. P., Ba, J.: Adam: A Method for Stochastic Optimization. In: Bengio, Y., LeCun, Y. (eds.) 3rd International Conference on Learning Representations, ICLR 2015, San Diego, CA, USA, May 7-9, 2015, Conference Track Proceedings (2017)
[36] Ayaz, M., Pasha, M. F., Alzahrani, M. Y., Budiarto, R., Stiawan, D.: The Fast Health Interoperability Resources (FHIR) Standard: Systematic Literature Review of Implementations, Applications, Challenges and Opportunities. JMIR medical informatics 9(7), 21929 (2021). https://doi.org/10.2196/21929
[37] Pedregosa, F., Varoquaux, G., Gramfort, A., Michel, V., Thirion, B., Grisel, O., Blondel, M., Müller, A., Nothman, J., Louppe, G., Prettenhofer, P., Weiss, R., Dubourg, V., Vanderplas, J., Passos, A., Cournapeau, D., Brucher, M., Perrot, M., Duchesnay, E.: Scikit-Learn: Machine Learning in Python. arXiv (2018)
[38] Aligning Books and Movies: Towards Story-like Visual Explanations by Watching Movies and Reading Books. 2015 IEEE International Conference on Computer Vision (ICCV), 19-27 (2015)
[39] Wikimedia Foundation: Wikimedia Downloads. https://dumps.wikimedia.org/
[40] pubmed.gov: Download PubMed Data. NCBI Literature Resources. https://pubmed.ncbi.nlm.nih.gov/download/
[41] PubMed Central: PMC Article Datasets. National Library of Medicine. https://www.ncbi.nlm.nih.gov/pmc/tools/textmining/
[42] Yang, X., PourNejatian, N., Shin, H. C., Smith, K. E., Parisien, C., Compas, C., Martin, C., Flores, M. G., Zhang, Y., Magoc, T., Harle, C. A., Lipori, G., Mitchell, D. A., Hogan, W. R., Shenkman, E. A., Bian, J., Wu, Y.: GatorTron: A Large Clinical Language Model to Unlock Patient Information from Unstructured Electronic Health Records. arXiv (2022)
[43] Shoeybi, M., Patwary, M., Puri, R., LeGresley, P., Casper, J., Catanzaro, B.: Megatron-LM: Training Multi-Billion Parameter Language Models Using Model Parallelism. arXiv (2020)
[44] Liaw, R., Liang, E., Nishihara, R., Moritz, P., Gonzalez, J. E., Stoica, I.: Tune: A Research Platform for Distributed Model Selection and Training. arXiv (2018)
[45] Welch, B. L.: THE GENERALIZATION OF ‘STUDENT'S’ PROBLEM WHEN SEVERAL DIFFERENT POPULATION VARLANCES ARE INVOLVED. Biometrika 34(1-2), 28-35 (1947). https://doi.org/10.1093/biomet/34.1-2.28
[46] Lin, Y.-W., Zhou, Y., Faghri, F., Shaw, M. J., Campbell, R. H.: Analysis and prediction of unplanned intensive care unit readmission using recurrent neural networks with long short-term memory. PLOS ONE 14(7), 0218942 (2019). https://doi.org/10.1371/journal.pone.0218942
[47] Gallagher, D., Zhao, C., Brucker, A., Massengill, J., Kramer, P., Poon, E. G., Goldstein, B. A.: Implementation and Continuous Monitoring of an Electronic Health Record Embedded Readmissions Clinical Decision Support Tool. Journal of Personalized Medicine 10(3), 103 (2020). https://doi.org/10.3390/jpm10030103
[48] Boag, W., Kovaleva, O., McCoy, T. H., Rumshisky, A., Szolovits, P., Perlis, R. H.: Hard for humans, hard for machines: Predicting readmission after psychiatric hospitalization using narrative notes. Translational Psychiatry 11(1), 32 (2021). https://doi.org/10.1038/s41398-020-01104-w
[49] Orangi-Fard, N., Akhbardeh, A., Sagreiya, H.: Predictive Model for ICU Readmission Based on Discharge Summaries Using Machine Learning and Natural Language Processing. Informatics 9(1), 10 (2022). https://doi.org/10.3390/informatics9010010
[50] Rajkomar, A., Oren, E., Chen, K., Dai, A. M., Hajaj, N., Hardt, M., Liu, P. J., Liu, X., Marcus, J., Sun, M., Sundberg, P., Yee, H., Zhang, K., Zhang, Y., Flores, G., Duggan, G. E., Irvine, J., Le, Q., Litsch, K., Mossin, A., Tansuwan, J., Wang, D., Wexler, J., Wilson, J., Ludwig, D., Volchen-boum, S. L., Chou, K., Pearson, M., Madabushi, S., Shah, N. H., Butte, A. J., Howell, M. D., Cui, C., Corrado, G. S., Dean, J.: Scalable and accurate deep learning with electronic health records. npj Digital Medicine 1(1), 18 (2018). https://doi.org/10.1038/s41746-018-0029-1
[51] Huang, K., Altosaar, J., Ranganath, R.: ClinicalBERT: Modeling Clinical Notes and Predicting Hospital Readmission. arXiv (2020)
[52] Weiss, A., Jiang, H.: Overview of Clinical Conditions With Frequent and Costly Hospital Readmissions by Payer, 2018. Agency for Healthcare Research and Quality, Rockville, MD (2021). https://pubmed.ncbi.nlm.nih.gov/34460186/
[53] The World Bank: Population, total. https://data.worldbank.org/indicator/SP.POP.TOTL
[54] OECD: Health at a Glance 2019: OECD Indicators (2019). https://doi.org/10.1787/4dd50c09-en
[55] McIlvennan, C. K., J., E. Z., A., A. L.: Hospital readmissions reduction program. Circulation 131(20), 1796-1803 (2015). https://doi.org/10.1161/CIRCULATIONAHA.114.010270
[56] Alsentzer, E., Murphy, J. R., Boag, W., Weng, W.-H., Jin, D., Naumann, T., McDermott, M.B.A.: Publicly Available Clinical BERT Embeddings. arXiv (2019)
[57] Chen, P.-H. C., Liu, Y., Peng, L.: How to develop machine learning models for healthcare. Nature Materials 18(5), 410-414 (2019). https://doi.org/10.1038/s41563-019-0345-0
[58] Matheny, M. E., Whicher, D., Thadaney Israni, S.: Artificial Intelligence in Health Care: A Report From the National Academy of Medicine. JAMA 323(6), 509 (2020). https://doi.org/10.1001/jama.2019.21579
[59] Yu, K.-H., Kohane, I. S.: Framing the challenges of artificial intelligence in medicine. BMJ Quality & Safety 28(3), 238-241 (2019). https://doi.org/10.1136/bmjgs-2018-008551
[60] Rajkomar, A., Dean, J., Kohane, I.: Machine Learning in Medicine. New England Journal of Medicine 380(14), 1347-1358 (2019). https://doi.org/10.1056/NEJMra1814259
[61] Xiao, C., Choi, E., Sun, J.: Opportunities and challenges in developing deep learning models using electronic health records data: A systematic review. Journal of the American Medical Informatics Association 25(10), 1419-1428 (2018). https://doi.org/10.1093/jamia/ocy068
[62] Campbell, C. M., Edwards, R. R.: Ethnic differences in pain and pain management. Pain Management 2(3), 219-230 (2012). https://doi.org/10.2217/pmt.12.7
[63] Zhu, X., Wong, F., Bensoussan, A., Lo, S. K., Zhou, C., Yu, J.: Are there any cross-ethnic differences in menstrual profiles?A pilot comparative study on Australian and Chinese women with primary dysmenorrhea: Ethnic differences in menstrual profiles. Journal of Obstetrics and Gynaecology Research 36(5), 1093-1101 (2010). https://doi.org/10.1111/j.1447-0756.2010.01250.x
[64] Barnett, M. L., Hsu, J., McWilliams, J. M.: Patient Characteristics and Differences in Hospital Readmission Rates. JAMA Internal Medicine 175(11), 1803 (2015). https://doi.org/10.1001/jamainternmed.2015.4660.
[65] Emily Alsentzer, John Murphy, William Boag, Wei-Hung Weng, Di Jindi, Tristan Naumann, and Matthew McDermott. 2019. Publicly Available Clinical BERT Embeddings. In Proceedings of the 2nd Clinical Natural Language Processing Workshop, pages 72-78, Minneapolis, Minnesota, USA. Association for Computational Linguistics.
[66] Tian Bai and Slobodan Vucetic. 2019. Improving Medical Code Prediction from Clinical Text via Incorporating Online Knowledge Sources. In The World Wide Web Conference, WWW '19, pages 72-82, New York, NY, USA. Association for Computing Machinery.
[67] Iz Beltagy, Matthew E. Peters, and Arman Cohan. 2020. Longformer: The Long-Document Transformer. ArXiv:2004.05150 [cs].
[68] Paula Branco, Luís Torgo, and Rita P. Ribeiro. 2016. A Survey of Predictive Modeling on Imbalanced Domains. ACM Computing Surveys, 49(2):31:1-31:50.
[69] Jacob Devlin, Ming-Wei Chang, Kenton Lee, and Kristina Toutanova. 2019. BERT: Pre-training of Deep Bidirectional Transformers for Language Understanding. In Proceedings of the 2019 Conference of the North American Chapter of the Association for Computational Linguistics: Human Language Technologies, Volume 1 (Long and Short Papers), pages 4171-4186, Minneapolis, Minnesota. Association for Computational Linguistics.
[70] C. Ferri, J. Hernández-Orallo, and R. Modroiu. 2009. An experimental comparison of performance measures for classification. Pattern Recognition Letters, 30(1):27-38.
[71] Chao-Wei Huang, Shang-Chi Tsai, and Yun-Nung Chen. 2022. PLM-ICD: Automatic ICD Coding with Pretrained Language Models. In Proceedings of the 4th Clinical Natural Language Processing Workshop, pages 10-20, Seattle, WA. Association for Computational Linguistics.
[72] Alistair E. W. Johnson, Tom J. Pollard, Lu Shen, Li-wei H. Lehman, Mengling Feng, Mohammad Ghassemi, Benjamin Moody, Peter Szolovits, Leo Anthony Celi, and Roger G. Mark. 2016. MIMIC-III, a freely accessible critical care database. Scientific Data, 3(1):160035. Number: 1 Publisher: Nature Publishing Group.
[73] Jinhyuk Lee, Wonjin Yoon, Sungdong Kim, Donghyeon Kim, Sunkyu Kim, Chan Ho So, and Jaewoo Kang. 2020. BioBERT: a pre-trained biomedical language representation model for biomedical text mining. Bioinformatics, 36(4):1234-1240.
[74] Fei Li and Hong Yu. 2020. ICD Coding from Clinical Text Using Multi-Filter Residual Convolutional Neural Network. Proceedings of the AAAI Conference on Artificial Intelligence, 34(05):8180-8187. Number: 05.
[75] Yinhan Liu, Myle Ott, Naman Goyal, Jingfei Du, Mandar Joshi, Danqi Chen, Omer Levy, Mike Lewis, Luke Zettlemoyer, and Veselin Stoyanov. 2019. RoBERTa: A Robustly Optimized BERT Pretraining Approach. ArXiv:1907.11692 [cs].
[76] Junyu Luo, Cao Xiao, Lucas Glass, Jimeng Sun, and Fenglong Ma. 2021. Fusion: Towards Automated ICD Coding via Feature Compression. In Findings of the Association for Computational Linguistics: ACLIJCNLP 2021, pages 2096-2101, Online. Association for Computational Linguistics.
[77] James Mullenbach, Sarah Wiegreffe, Jon Duke, Jimeng Sun, and Jacob Eisenstein. 2018. Explainable Prediction of Medical Codes from Clinical Text. In Proceedings of the 2018 Conference of the North American Chapter of the Association for Computational Linguistics: Human Language Technologies, Volume 1 (Long Papers), pages 1101-1111, New Orleans, Louisiana. Association for Computational Linguistics.
[78] Damian Pascual, Sandro Luck, and Roger Wattenhofer. 2021. Towards BERT-based Automatic ICD Coding: Limitations and Opportunities. In Proceedings of the 20th Workshop on Biomedical Language Processing, pages 54-63, Online. Association for Computational Linguistics.
[79] Ines Rieger, Jaspar Pahl, Bettina Finzel, and Ute Schmid. 2022. CorrLoss: Integrating Co-Occurrence Domain Knowledge for Affect Recognition. In 2022 26th International Conference on Pattern Recognition (ICPR), pages 798-804. ISSN: 2831-7475.
[80] Yanmin Sun, Andrew K. C. Wong, and Mohamed S. Kamel. 2009. Classification of imbalanced data: a review. International Journal of Pattern Recognition and Artificial Intelligence, 23(04):687-719. Publisher: World Scientific Publishing Co.
[81] Fei Teng, Wei Yang, Li Chen, LuFei Huang, and Qiang Xu. 2020. Explainable Prediction of Medical Codes With Knowledge Graphs. Frontiers in Bioengineering and Biotechnology, 8.
[82] Shang-Chi Tsai, Ting-Yun Chang, and Yun-Nung Chen. 2019. Leveraging Hierarchical Category Knowledge for Data-Imbalanced Multi-Label Diagnostic Text Understanding. In Proceedings of the Tenth International Workshop on Health Text Mining and Information Analysis (LOUHI 2019), pages 39-43, Hong Kong. Association for Computational Linguistics.
[83] Thanh Vu, Dat Quoc Nguyen, and Anthony Nguyen. 2020. A Label Attention Model for ICD Coding from Clinical Text. volume 4, pages 3335-3341. ISSN: 1045-0823.
[84] Ke Wang, Xuyan Chen, Ning Chen, and Ting Chen. 2020. Automatic Emergency Diagnosis with Knowledge-Based Tree Decoding. volume 4, pages 3407-3414. ISSN: 1045-0823.
[85] Chenwei Yan, Xiangling Fu, Xien Liu, Yuanqiu Zhang, Yue Gao, Ji Wu, and Qiang Li. 2022. A survey of automated International Classification of Diseases coding: development, challenges, and applications. Intelligent Medicine, 2(3):161-173.
[86] Ying Yu, Min Li, Liangliang Liu, Zhihui Fei, Fang-Xiang Wu, and Jianxin Wang. 2019. Automatic ICD code assignment of Chinese clinical notes based on multilayer attention BiRNN. Journal of Biomedical Informatics, 91:103114.
[87] Min Zeng, Min Li, Zhihui Fei, Ying Yu, Yi Pan, and Jianxin Wang. 2019. Automatic ICD-9 coding via deep transfer learning. Neurocomputing, 324:43-50.
[88] Zachariah Zhang, Jingshu Liu, and Narges Razavian. 2020. BERT-XML: Large Scale Automated ICD Coding Using BERT Pretraining. In Proceedings of the 3rd Clinical Natural Language Processing Workshop, pages 24-34, Online. Association for Computational Linguistics.
[89] Zhi-Hua Zhou and Xu-Ying Liu. 2006. Training costsensitive neural networks with methods addressing the class imbalance problem. IEEE Transactions on Knowledge and Data Engineering, 18(1):63-77. Conference Name: IEEE Transactions on Knowledge and Data Engineering.
[90] Quan Zou, Sifa Xie, Ziyu Lin, Meihong Wu, and Ying Ju. 2016. Finding the Best Classification Threshold in Imbalanced Classification. Big Data Research, 5:2-8.
[91] Emily Alsentzer, John R. Murphy, Willie Boag, Wei-Hung Weng, Di Jin, Tristan Naumann, and Matthew B. A. McDermott. 2019. Publicly available clinicalbert embeddings.
[92] Iz Beltagy, Matthew E. Peters, and Arman Cohan. 2020. Longformer: The long-document transformer. CoRR, abs/2004.05150.
[93] Sajad Darabi, Mohammad Kachuee, Shayan Fazeli, and Majid Sarrafzadeh. 2020. Taper: Time-aware patient ehr representation. IEEE Journal of Biomedical and Health Informatics, 24(11):3268-3275.
[94] Kexin Huang, Jaan Altosaar, and Rajesh Ranganath. 2019. Clinicalbert: Modeling clinical notes and predicting hospital readmission. arXiv preprint arXiv:1904.05342.
[95] Alistair E. W. Johnson, Tom J. Pollard, Lu Shen, Li wei H. Lehman, Mengling Feng, Mohammad Ghassemi, Benjamin Moody, Peter Szolovits, Leo Anthony Celi, and Roger G. Mark. 2016. Mimic-iii, a freely accessible critical care database. Nature.
[96] Yikuan Li, Ramsey M. Wehbe, Faraz S. Ahmad, Hanyin Wang, and Yuan Luo. 2022. Clinical-longformer and clinical-bigbird: Transformers for long clinical sequences. CoRR, abs/2201.11838.
[97] Sebastian Schneeweiss, John D Seeger, Malcolm Maclure, Philip S Wang, Jerry Avorn, and Robert J Glynn. 2001. Performance of comorbidity scores to control for confounding in epidemiologic studies using claims data. American journal of epidemiology, 154(9):854-864.
[98] Nischay Bikram Thapa, Sattar Seifollahi, and Sona Taheri. 2022. Hospital readmission prediction using clinical admission notes. In Australasian Computer Science Week 2022, pages 193-199.
[99] Grace Yang, Ming Cao, Lavender Y Jiang, Xujin C Liu, Alexander Cheung, Hannah Weiss, Davied Kurland, Kyunghyun Cho, and Eric K Oermann. 2022. Language model classifier aligns better with physician word sensitivity than xgboost on readmission prediction. arXiv preprint arXiv:2211.07047.

1. A method for generating at least one medical prediction, comprising:
converting, by at least one computer processor, clinical notes to training data using at least one natural language processing procedure;
training, by the at least one computer processor, a machine learning model using the training data;
finetuning, by the at least one computer processor, the trained machine learning model based on selected parameters;
receiving patient data; and
generating, by the at least one computer processor, the at least one medical prediction on the received patient data with the trained finetuned machine learning model.

2. The method of claim 1, wherein the clinical notes include at least one of (i) structured data and unstructured data, or (ii) discharge notes.

3. The method of claim 1, further comprising:
integrating the trained finetuned machine learning model in real-time with clinical workflows.

4. The method of claim 1, wherein the machine learning model is trained using non-clinical data.

5. The method of claim 1, wherein the at least one medical prediction includes information associated with a readmission to a hospital.

6. (canceled)

7. The method of claim 1, wherein the trained machine learning model is finetuned by replacing the trained machine learning model with a randomly initialized linear classifier after a last hidden layer of a pretrained BERT model.

8. A system for generating at least one medical prediction, comprising:
at least one computer processor which is configured to:
convert clinical notes to training data using a natural language processing procedure;
train a machine learning model using the training data;
finetune the trained machine learning model based on selected parameters;
receive patient data; and
generate the at least one medical prediction on the received patient data with the trained finetuned machine learning model.

9. The system of claim 8, wherein the clinical notes include at least one of (i) structured data and unstructured data, or (ii) discharge notes.

10. The system of claim 8, wherein the at least one computer processor is further configured to:
integrate the trained finetuned machine learning model in real-time with clinical workflows.

11. The system of claim 8, wherein the at least one computer processor is further configured to train the machine learning model using non-clinical data.

12. The system of claim 8, wherein the at least one medical prediction includes information associated with a readmission to a hospital.

13. (canceled)

14. The system of claim 8, wherein the finetuning includes replacing the trained machine learning model with a randomly initialized linear classifier after a last hidden layer of a pretrained BERT model.

15. A computer accessible medium which includes software thereon for generating at least one medical prediction, wherein, when at least one computer processor execute the software, the computer processor is configured to perform the procedures, comprising:
converting clinical notes to training data using a natural language processing procedure;
training a machine learning model using the training data;
finetuning the trained machine learning model based on selected parameters;
receiving patient data; and
generating the at least one medical prediction on the received patient data with the trained finetuned machine learning model.

16. The computer accessible medium of claim 15, wherein the clinical notes include at least one of (i) structured data and unstructured data, or (ii) discharge notes.

17. The computer accessible medium of claim 15, further comprising:
integrating the trained finetuned machine learning model in real-time with clinical workflows.

18. The computer accessible medium of claim 15, wherein the machine learning model is trained using non-clinical data.

19. The computer accessible medium of claim 15, wherein the at least one medical prediction includes information associated with a readmission to a hospital.

20. (canceled)

21. The computer accessible medium of claim 15, wherein the trained machine learning model is finetuned by replacing the trained machine learning model with a randomly initialized linear classifier after a last hidden layer of a pretrained BERT model.

22. A system for generating a table language, comprising:
a computer processor implementing an artificial intelligence model configured to generate code to create a structured database procedure.

23. The system of claim 22, wherein the code is generated by the artificial intelligence model to create the structured database procedure, and wherein the code cases the computer processor to convert unstructured text into a plurality of SQL tables.

24. The system of claim 23, wherein the unstructured text comprises electronic health records free text.

25. A method for generating a table language, comprising:
generating, with an artificial intelligence model operating on a computer processor, code to create a structured database procedure.

26-30. (canceled)

31. A system for training an electronic health records (EHR) artificial intelligence model, comprising:
a computer processor configured to train the EHR artificial intelligence model on a training data set that comprises a plurality of EHR records utilizing an under-sampling technique.

32. The system of claim 31, wherein the under-sampling technique comprises at least one of (i) an iterative summation, (ii) a hierarchy, or (iii) a sparse-attention model.

33. The system of claim 32, wherein the iterative summation comprises a procedure which:
selects, by the computer processor, a fixed amount of data from a selected one of the plurality of EHR records;
summarizes, by the computer processor, information in the fixed amount of data;
selects, by the computer processor, a next fixed amount of data from the selected EHR record;
feeds, by the processor, the summary and the next fixed amount of data back into the EHR artificial intelligence model; and
creates, by the processor, an updated summary based on the summary and next fixed amount of data.

34. (canceled)

35. The system of claim 32, wherein the hierarchy comprises a procedure which:
selects, by the computer processor, a first fixed amount of data from a selected one of the plurality of EHR records;
converts, by the computer processor, the first fixed amount of data into a machine language;
selects, by the computer processor, a second fixed amount of data from the selected EHR record; and
converts, by the computer processor, the second fixed amount of data into a machine language that is added to the machine language for the first fixed amount of data.

36. (canceled)

37. The system of claim 32, wherein the sparse-attention model comprises a procedure which:
selects, by the computer processor, a word sampling rate for the plurality of EHR records;
applies, by the computer processor, the word sampling rate to the plurality of EHR records; and
trains, by the computer processor, the EHR artificial intelligence model on the plurality of EHR records subject to the word sampling rate.

38. A method for training an electronic health records (EHR) artificial intelligence model, comprising:
training the EHR artificial intelligence model on a training data set that comprises a plurality of EHR records utilizing an under-sampling technique.

39-51. (canceled)
